(19) United States
(12) Patent Application Publication
(10) Pub. No.: US 2022/0145380 A1
(43) Pub. Date: May 12, 2022

(54) COST-EFFECTIVE DETECTION OF LOW FREQUENCY GENETIC VARIATION

(71) Applicant: CHILDREN'S MEDICAL CENTER CORPORATION, Boston, MA (US)

(72) Inventors: Ryan N. DOAN, Boston, MA (US); Christopher A. WALSH, Boston, MA (US)

(73) Assignee: CHILDREN'S MEDICAL CENTER CORPORATION, Boston, MA (US)

(21) Appl. No.: 17/427,394

(22) PCT Filed: Nov. 26, 2019
(86) PCT No.: PCT/US2019/063447

§ 371 Date: Jul. 30, 2021

Related U.S. Application Data
(60) Provisional application No. 62/799,671, filed on Jan. 31, 2019.

(51) Int. Cl. C12Q 1/6851; C12Q 1/6886

(52) U.S. Cl. CPC C12Q 1/6851; C12Q 2600/156; C12Q 2600/118; C12Q 1/6886

(57) ABSTRACT

Methods are described for the detection of low frequency genetic variants, such as somatic mosaic variants. The methods comprise parallel amplification reactions of a target nucleic acid sequence to generate overlapping amplicons, pooled sequencing of the amplicons, and demultiplexed detection of low frequency variants.

### CROSS-REFERENCE TO RELATED APPLICATION

This application claims the benefit of the following U.S. Provisional Application No. 62/799,671, filed Jan. 31, 2019, the entire contents of which are incorporated herein by reference.

### STATEMENT OF RIGHTS TO INVENTIONS MADE UNDER FEDERALLY SPONSORED RESEARCH

This invention was made with government support under Grant Nos. R01NS032457 and U01MH106883 awarded by the National Institutes of Health. The government has certain rights in the invention.

### BACKGROUND OF THE INVENTION

Traditional genetic sequencing methodologies, such as whole genome (WGS) and whole exome (WES), have focused on the important contribution of germline mutations that are present in all cells throughout the human body. However, recent studies have shown numerous examples of mutations occurring after fertilization (i.e. postzygotic mutations), which are only present in a fraction of the cells. Postzygotic mutations, or somatic mutations, have been heavily studied in cancers where clinical diagnostic testing for somatic mutations in tumor and blood samples are becoming a standard practice due to improved detection sensitivities when most cells in the sample carry a given mutation.

Beyond technical errors, an important consideration for skewed alternate allelic fraction (AAFs), false negatives, and false positives are allelic imbalances caused by inherent differences in the genome content around a mutation. These issues, such as additional mutations, repeat content, methylation, or copy number changes, can have dramatic impacts on AAFs, resulting in the commonly recognized issue of allelic dropout. To avoid allelic dropout, many methods avoid placing primers in areas with known genetic variation in the general population. However, these methods remain susceptible to allelic skewing from ultra-rare or private alleles and other loci specific causes of allelic imbalance. Cost-effective methods are needed for the detection and characterization of rare alleles and other genetic variants.

### SUMMARY OF THE INVENTION

As described below, the present disclosure features methods for detecting and quantifying genetic variants in a sample.

In one aspect of the present disclosure, a method is provided for determining alternate allele frequency, the method involves performing two or more parallel amplification reactions on a single sample, thereby generating overlapping amplicons, where each amplification reaction includes a unique pair of forward and reverse primers, where the forward or reverse primer includes an index sequence, and where the forward and reverse primers include different adapter sequences. The method also involves sequencing the overlapping amplicons to produce sequence reads, segregating the sequencing reads into bins by index sequence, and detecting the presence or absence of one or more genetic variants within sequencing reads within a bin, where the frequency of detection of the variant determines the alternate allele frequency.

Another aspect provides a method for determining alternate allele frequency, the method involves a) performing three amplification reactions on a single sample, thereby generating three overlapping amplicons, where each amplification reaction includes a unique pair of forward and reverse primers, where each primer includes a nucleic acid sequence complementary to a portion of a target nucleic acid sequence, where the forward or reverse primer includes an index sequence, where the forward and reverse primers include different adapter sequences at or near the 5′ terminus of the primer and upstream of the sequence complementary to the target, and where at least one adapter sequence is complementary to a nucleic acid sequence used in sequencing; b) sequencing the overlapping amplicons to produce sequence reads; c) segregating the sequencing reads into bins by index sequence; and d) detecting the presence or absence of one or more genetic variants within sequencing reads within a bin, where the frequency of detection of the variant determines the alternate allele frequency.

Another aspect of the present invention provides a method for method for determining alternate allele frequency, the method involving a) performing three amplification reactions on a single sample, thereby generating three overlapping amplicons, where each amplification reaction includes a unique pair of forward and reverse primers, where the forward or reverse primer comprises an index sequence and/or a unique molecular identifier (UMI); and each primer includes i. a nucleotide sequence complementary to a portion of a target nucleic acid sequence; ii. an adapter at or near its 5′ terminus, where the adapter is upstream of the sequence complementary to the target and wherein the forward and reverse primers include different adapter sequences, and where at least one adapter sequence is complementary to a nucleic acid sequence used in sequencing; b) sequencing the overlapping amplicons to produce sequence reads; c) segregating the sequencing reads into bins by index sequence; d) detecting the UMI and removing duplicate reads from the bin, where the detecting can be simultaneous with step c or subsequent to step c; and e) detecting the presence or absence of one or more genetic variants within sequencing reads within a bin, where the frequency of detection of the variant determines the alternate allele frequency.

In some embodiments, the methods disclosed herein further involve pooling the amplicons prior to sequencing. In some embodiments of the methods disclosed herein, sequencing the amplicons involves contacting the amplicons with a nucleic acid complementary to the adapter sequence. In some embodiments, the amplicons include a nucleotide having a label, and in some embodiments, the label is biotin. In some embodiments, the methods disclosed herein also involve contacting the label with a capture agent that specifically binds the label. In some embodiments, the methods also involve enzymatically digesting the primers. In some embodiments of the present disclosure, the methods also involve amplifying the amplicons, thereby generating enriched populations of amplicons. In some embodiments, the genetic variation to be detected is known or unknown. In some embodiments, the genetic variant has an alternate allele fraction of at least 0.1%. In some embodiments, the genetic variant has an alternate allele fraction of at least 0.025%. In some embodiments, the genetic variant is a mosaic variant. In some embodiments, detection of the genetic variant identifies the presence of a disease or a predisposition to a disease in a subject from whom the sample was derived. In some embodiments, the disease is cancer. In some embodiments, the sample includes circulating tumor cells or cell free DNA. In some embodiments, the genetic variant originated from a somatic event or a germline event. In some embodiments, the alternate allele frequency is compared to the allele frequency of a reference sample to determine if the subject's disease is progressing, regressing, or in remission. In some embodiments, the methods further involve averaging the alternate allele frequencies determined for each bin. In some embodiments, the methods further involve determining the error rate of the nucleic acid sequences flanking the alternate allele.

Methods defined by the present disclosure were performed in connection with the examples provided below. Other features and advantages of the disclosure will be apparent from the detailed description and from the claims.

### Definitions

Unless defined otherwise, all technical and scientific terms used herein have the meaning commonly understood by a person skilled in the art to which this disclosure relates. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, “adapter” refers to a nucleic acid sequence in an amplification primer that is complementary to the sequence of a nucleic acid molecule used to prime downstream sequencing reactions.

The term “allelic dropout” refers to the loss of one allele during amplification, resulting in apparent homozygosity. Nucleotide variation, cytosine methylation, and nucleic acid structure in the primer binding site of only one allele can cause allelic dropout when primer binding to the to the primer binding site is inhibited or reduced. For example, G-quadruplexes (secondary structures formed from stacks of G-quartets) present in the primer binding sites of an allele can prevent efficient priming of the template nucleic acid and lead to allelic dropout.

By “alternative allele” is meant an allele other than a reference allele. An alternative allele will have genetic variation that is not present in the reference allele. In some embodiments, a reference allele is a wildtype allele. A reference allele may differ between different populations, races, or ethnicities. Genetic variation present in an alternative allele can be nucleotide variation (i.e., a transition or a transversion), an insertion, or a deletion. An alternative allele may have a silent variant or mutation, a missense variant or mutation, or a nonsense variant or mutation.

By “alternative allele fraction” is meant the frequency of an allele, other than a reference allele, in a population of cells in an individual. The alternative allele fraction is often less than that of the reference allele fraction, especially when the reference allele is a wildtype allele.

By “amplicon” is meant the product of an amplification reaction.

By “amplification bias” is meant a tendency for a nucleic acid amplification reaction to yield a particular amplicon. Amplification bias is often associated with inefficient primer binding. For example, if a primer's nucleic acid sequence is less complementary to the sequence of a template nucleic acid, the primer will be less likely to bind to the template than a primer having a more complementary sequence. Variants present in the primer binding site of a template nucleic acid may result in conformational or structural changes to the nucleic acid molecule that inhibit primer binding. Other variants or modifications (e.g., methylated nucleic acid residues) present in the primer binding site or elsewhere in the nucleic acid molecule can also cause to amplification bias. Amplification bias may result in underrepresentation of an allele or allelic dropout.

By “analog” is meant a molecule that is not identical, but has analogous functional or structural features to a naturally occurring molecule. For example, a polynucleotide analog retains the biological activity of a corresponding naturally-occurring polynucleotide while having certain modifications that enhance the analog's function relative to a naturally occurring polynucleotide. Such modifications could increase the polynucleotide's affinity for DNA, half-life, and/or nuclease resistance, an analog may include an unnatural nucleotide or amino acid.

By “bin” is meant a collection of sequencing reads that are substantially identical. In some instances, a bin comprises sequences reads that have the same index sequence or UMI sequence.

The phrase “biological sample” as used herein refers to a sample taken from a biological source and includes, but is not limited to, blood, serum, plasma, sputum, lavage fluid, cerebrospinal fluid, urine, semen, sweat, tears, tissue biopsy, and saliva. As used herein, the terms “blood,” “plasma,” and “serum” expressly encompass fractions or processed portions thereof.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “demultiplex” is meant a process in which sequence reads generated from different amplicons are segregated into groups based on at least one characteristic unique to each group. For example, the index sequence of a primer can be used to segregate the sequence reads.

The term “denaturing,” as contemplated herein, refers to removing impediments to primer binding from a nucleic acid. For example, denaturing includes removing conformational or structural properties of a nucleic acid or separating a nucleic acid duplex into single strands. Denaturing is facilitated by exposing the duplex to at least one denaturing condition or agent. Denaturing conditions are well known in the art. In one embodiment, a nucleic acid duplex is denatured by exposing it to a temperature that is above the melting temperature (Tm) of the duplex. In certain embodiments, a nucleic acid may be denatured by exposing it to a temperature of at least 90° C. for a sufficient amount of time to denature the nucleic acid molecule. In some embodiments, a denaturing agent may include a chemical additive that facilitates denaturation, for example, sodium hydroxide or urea.

“Detect” refers to discovering or identifying the presence, absence, or amount of an analyte (e.g., genetic variation) to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

“DMSO” refers to dimethyl sulfoxide, which has the following structure:

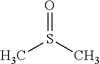

The term “enrich,” as used herein, refers to the process of further amplifying nucleic acid amplicons. In some embodiments, enrichment of nucleic acid amplicon allows for more efficient detection and quantifying of genetic variants having very low alternative allele frequency relative to detecting and quantifying genetic variants with very low alternative allele frequency in non-enriched nucleic acid amplicons.

By “GC buffer” is meant a reagent designed to optimize the ionic environment of an amplification reaction of a nucleic acid molecule having an enriched guanine/cytosine sequence.

“Germline allele” means an allele specific to germ cells or progenitors thereof.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “index sequence” or “barcode” is meant a portion of a nucleic acid molecule that allows grouping or demultiplexing of sequencing reads. For example, an index sequence enables the segregation of sequence reads into bins, wherein each bin comprises sequence reads of amplicons generated from the primer pair having the index sequence. In some embodiments, each primer pair used in the presently disclosed methods has a unique index sequence.

As used herein, “interrogate” refers to obtaining nucleotide sequence information for a nucleic acid molecule.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” nucleic acid is sufficiently free of other materials such that any impurities do not materially affect the biological properties of the nucleic acid or cause other adverse consequences. That is, a nucleic acid of this disclosure is purified if it is substantially free of cellular material, viral material, or culture medium. Purity and homogeneity are typically determined using analytical chemistry techniques, for example, polyacrylamide gel electrophoresis or high-performance liquid chromatography. The term “purified” can denote that a nucleic acid gives rise to essentially one band in an electrophoretic gel.

By “isolated polynucleotide” is meant a nucleic acid (e.g., a DNA) that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

“Isothermal” refers to a process incubated at about a constant temperature. For example, some isothermal amplification reactions are carried out at about 65° C. An isothermal temperature may depart from an intended temperature by not more than about 10% or 5° C., whichever is greater. An isothermal reaction may include an initial incubation at a higher temperature (“a hot start”). A hot start may comprise incubating the amplification reaction at a temperature sufficient to denature a region of interest on a nucleic acid molecule or to active a reagent (i.e., a polymerase).

By “marker” is meant any protein or polynucleotide associated with a disease or disorder.

As used herein, “mosaic” refers to two or more cells or populations of cells with different genotypes within an individual subject. For example, “somatic mosaicism” refers to two or more genotypically distinct somatic cells or populations of somatic cells in an individual. “Germline mosaicism” occurs when two or more genotypically distinct germ cells or populations of germ cells are present in an individual. Germline mosaicism generally arises after a mutation gives rise to a genotypically distinct gamete.

The term “Next Generation Sequencing (NGS)” refers to massive parallel sequencing of clonally amplified molecules or single nucleic acid molecules. “Massive parallel sequencing” refers to simultaneously performing more than 1000 separate, parallel sequencing reactions. Non-limiting examples of NGS include sequencing-by-synthesis using reversible dye terminators, sequencing-by-ligation, and electronic detection sequencing methods. Electronic detection sequencing methods include those used in the Ion Torrent sequencing strategy (ThermoFisher Scientific) or MiSeq platform (Illumina), wherein changes in pH are detected when a nucleotide is incorporated into a nucleic acid strand resulting in release of a hydrogen ion.

The terms “nucleic acid” and “nucleic acid molecule,” are used interchangeably herein and refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acid molecules assayed using the methods described herein need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or about 250 mM NaCl and 25 mM trisodium citrate. Low stringency hybridization can be obtained in the absence of organic solvent, e.g., formamide, while high stringency hybridization can be obtained in the presence of at least about 35% formamide, and in some embodiments, at least about 50% formamide. Stringent temperature conditions will ordinarily include temperatures of at least about 30° C. at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In yet another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will comprise less than about 30 mM NaCl and 3 mM trisodium citrate or less than about 15 mM NaCl and 1.5 mM trisodium citrate. Stringent temperature conditions for the wash steps will ordinarily include a temperature of at least about 25° C., at least about 42° C., or at least about 68° C. In some embodiments, wash steps will occur at 25° C. in 30 mM NaCl, 3 mM trisodium citrate, and 0.1% SDS. In other embodiments, wash steps will occur at 42° C. in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

As used herein, “obtaining” as in “obtaining an agent” includes synthesizing, purchasing, or otherwise acquiring the agent.

By “overlapping amplicons” is meant two or more amplicons that comprise a shared nucleic acid sequence but have at least one different terminal sequence.

“Polymerase” refers to an enzyme capable of catalyzing nucleic acid synthesis. A polymerase can be a DNA polymerase or an RNA polymerase. A polymerase can be characterized by its error rate, or the rate at which the polymerase inserts an incorrect nucleotide into the nucleic acid molecule it is synthesizing. In some embodiments, a polymerase can be a high-fidelity polymerase, which has a much lower error rate than a reference polymerase. A non-limiting example of a reference polymerase is Taq polymerase.

“Pooling,” as used herein, means combining multiple amplification reactions or groups of reactions. Pooling is synonymous with multiplexing.

By “portion” is meant a segment of an intact nucleic acid molecule. This portion contains, in some embodiments, at least 10%, 20%, 30%, 40%, 50%, 60%, 70%, 80%, or 90% of the entire length of the reference nucleic acid molecule. A portion may contain 10, 20, 30, 40, 50, 60, 70, 80, 90, or 100, 200, 300, 400, 500, 600, 700, 800, 900, or 1000 nucleotides.

The term “read,” “sequence read,” or “sequencing read” refers to sequencing data from a region of a nucleic acid molecule obtained from a single nucleic acid molecule. A read represents a short sequence of contiguous bases in the nucleic acid molecule and may be depicted, for example, as a chromatogram or as a linear string of letters that represent the nitrogenous bases of the nucleotide sequence, wherein A=adenine; G=guanine; C=cytosine; T=thymine; U=uracil; R=purine (A or G); Y=pyrimidine (C or T); N=any nucleotide; W=A or T; S=G or C; K=G or T; B=Not A; H=Not G; D=Not C; and V=Not T.

“Reduces” or “increases” refers to a negative or positive alteration, respectively, of at least 10%, 25%, 50%, 75%, or 100%.

By “reference” is meant a standard or control condition.

A “reference sequence” is a defined sequence used for sequence comparison. A reference sequence may be a subset of or the entirety of a specified sequence; for example, a segment of a full-length gene sequence, or the complete gene sequence. For nucleic acids, the length of the reference nucleic acid sequence will generally be at least about 50 nucleotides, at least about 60 nucleotides, at least about 75 nucleotides, about 100 nucleotides, or even about 300, 400, or 500 nucleotides or any integer thereabout or therebetween. In some embodiments, the length of the reference nucleic acid sequence will be less than 50 nucleotides. In some embodiments, the reference nucleic acid sequence will be more than 500 nucleotides.

The term “sequence variant,” as used herein, refers to an alteration in a sequence relative to a reference sequence. In one embodiment, a nucleotide sequence variant comprises one or more alterations relative to a reference nucleotide sequence. In some embodiments, the reference sequence is a consensus sequence. Optimally aligned sequencing reads obtained from multiple individuals of the same species or a population thereof, or multiple sequencing reads for the same individual, may be used to produce a consensus sequence. As contemplated herein, a “consensus sequence” refers to a nucleotide sequence that comprises the base most in common among all the sequencing reads at each nucleotide in the sequence.

In some embodiments, a sequence variant represents a variation relative to corresponding sequences in the same sample. In some embodiments, the sequence variant occurs with a low frequency (i.e., at least <1%) in the population (also referred to as a “rare variant”). For example, the sequence variant may occur with a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. In some embodiments, the sequence variant occurs with a frequency above about 0.1%. In some embodiments, the sequence variant occurs at a frequency of above about 0.0025%.

By “somatic allele” is meant an allele specific to a non-germline cell (i.e., somatic cell).

By “somatic event” is meant the acquisition of a genetic variant by a somatic cell.

By “subject” is meant a mammal, including a human or a non-human mammal, such as a bovine, equine, canine, ovine, feline, or rodent (e.g., mouse, rat).

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 60%, 80% or 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and e⁻¹⁰⁰ indicating a closely related sequence.

The term “tissue” refers to a group or layer of similarly specialized cells, which together perform certain special functions. The term “tissue-specific” refers to a source or defining characteristic of cells from a specific tissue.

By “unique molecular identifier (UMI)” is meant a distinct nucleic acid sequence that individualizes each primer used in an amplification reaction. For example, 500 primers having identical complementary nucleic acid sequences will have 500 different UMIs. UMIs facilitate the detection and removal of redundant sequencing reads.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A to 1C are schematic diagrams illustrating the primer design strategy used in the presently disclosed methods. FIG. 1A is a schematic diagram illustrating overlapping amplicons that provide redundant coverage of a variant of interest (A/G). Primer 1, Primer 2, and Primer 3 refer to the pairs of forward and reverse primers (depicted at the termini of the intervening line). The intervening line represents the nucleic acid sequence to be amplified. “SNV” refers to single nucleotide variant. FIG. 1B is a schematic diagram of three amplicons, wherein “Adapter 1” and “Adapter 2” refer to the adapter sequences upstream from the primer's complementary nucleotide sequence (“Forward” or “Reverse”). Each reverse primer has one of three index sequences. FIG. 1C is a schematic diagram of three amplicons that comprise a unique molecular identifier (UMI).

FIG. 2 comprises three panels of aligned sequencing reads, wherein each panel comprises sequencing reads of amplicons generated from one of three amplification reactions. The top and bottom panels each show alternate allele fractions of a detected variant of approximately 50%. The middle panel shows an alternate allele fraction of only 3%, which indicates allelic dropout.

FIG. 3 is an illustration of capturing and enriching amplified nucleic acids.

FIG. 4 is a schematic diagram of a method for detecting low frequency variants in a nucleic acid molecule. Throughout the figures, QC denotes quality control and AAF denotes alternative allele fractions.

FIG. 5A is a schematic diagram of a method for detecting and characterizing low frequency variants. CI denotes confidence interval. FIG. 5B is a diagram illustrating an optional quality control step that can be added to the method depicted in FIG. 5A.

FIG. 6 is a chart summarizing an Ion Torrent Next Generation Sequencing run and the data generated therefrom.

FIG. 7 is an illustration of demultiplexing sequencing data.

FIG. 8 is data output illustrating sequencing errors generated using the Ion Torrent platform. Specifically, the data presented illustrates how sequencing errors (i.e., indels) are processed using the disclosed methods.

FIG. 9 is an illustration of sequencing reads, wherein the ends of each read (i.e., the primer sequences) are easily observed.

FIG. 10A is an illustration of the reproducibility observed in aligned sequencing data of a germline event. The illustration depicts three panels of aligned sequence data indicating the presence of a variant at base pair number 14,234,400. FIG. 10B is an illustration of a detected mutation.

FIGS. 11A to 11G graphically illustrate quality control assessment of amplification products generated using the methods as described herein. FIG. 11A is an electronically generated gel image of products of an amplification reaction performed according to the methods described herein. Lane (L) 1 comprises a control sample “Control-6-U” that was not amplified using the methods disclosed herein. Lane 2 comprises amplification products generated using a single amplification (20 cycles) protocol as described herein. Lane 3 comprises amplification products using a two-amplification protocol (first amplification=8 cycles; second amplification=20 cycles). “Bio” indicates the first-round amplification products were biotinylated. Lane 4 comprises amplification products generated using a two-amplification protocol (first amplification=10 cycles; second amplification=20 cycles). Lane 5 comprises amplification products generated using a two-amplification protocol (first amplification=10 cycles; second amplification=20 cycles). “Amp” indicates the first-round reaction products were not biotinylated. “[s]” refers to seconds. FIG. 11B is a graph illustrating the fluorescent peaks detected when analyzing the control reaction “Control-6-U” using the Bioanalyser 2100. FIG. 11C is a graph illustrating the fluorescent peaks detected when analyzing the “20X-Norm” reaction using the Bioanalyser 2100. FIG. 11D is a graph illustrating the fluorescent peaks detected when analyzing the “8X_20X_Bio” reaction using the Bioanalyser 2100. FIG. 11E is a graph illustrating the fluorescent peaks detected when analyzing the “10X_20X_Bio” reaction using the Bioanalyser 2100. FIG. 11F is a graph illustrating the fluorescent peaks detected when analyzing the “10X_20X_Amp” reaction using the Bioanalyser 2100. FIG. 11G is a graph illustrating the fluorescent peaks detected when analyzing the “Exo 8X_20X_RD” reaction using the Bioanalyser 2100. This reaction was purified using the ExoSAP protocol described herein after amplifying a target nucleic acid using a two-amplification protocol as used herein. In this sample, the target nucleic acid was amplified with a first reaction comprising 8 cycles and then a subsequent amplification reaction comprising 20 cycles.

FIG. 12 is a graph depicting a TapeStation analyzer's quality control assessment of the products generated in an amplification reaction. The “upper” and “lower” peaks are the control peaks, and the “283” peak represents the amplification reaction products.

FIG. 13 is a graph illustrating the accuracy and reproducibility of the present methods to detect variants and provide accurate alternative allele fractions.

FIG. 14 is a graph illustrating the accuracy and reproducibility of the present methods to detect low frequency variants and provide accurate alternative allele fractions (i.e., AAF<1%).

FIG. 15 is a graph of a deleterious missense mosaic variant detected in the CACNA1A gene of a single individual.

FIG. 16 is a graph of number of germline heterozygous single nucleotide having a particular variant (alternate) allele fraction (VAF).

FIGS. 17A to 17D are graphs and figures explaining asymmetric cell contribution. FIG. 17A is a graph showing asymmetrical cell contributions to brain development during early embryonic development. FIG. 17B is an illustration of the different branches of early phylogeny at which mutations may be acquired. FIG. 17C is a graph showing poor stability of the asymmetric parameter α₁ estimated from the 2nd cell generation compared to only one asymmetric cell division. FIG. 17D is a graph showing the confidence interval for the asymmetric cell contribution parameter.

FIGS. 18A-18D illustrate that the presently described methods accurately measure AAFs as low as 0.01% when using a 50 ng of genomic DNA. FIG. 18A is a graph showing the correlation of expected and measured AAFs up to 60% for samples comprising 50 ng of DNA. FIG. 18B is a graph showing the correlation of expected and measured AAFs between 0 and 1.0%. FIG. 18C is a graph showing the correlation of expected and measured AAFs up to 60% for samples comprising 25 ng of DNA. FIG. 18D is a graph showing the correlation of expected and measured AAFs between 0 and 1.0%.

FIG. 19A is a graph correlating the AAF's of single nucleotide variants determined using whole genome sequencing (WGS) and triple-primer PCR sequencing (Trip-Seq). FIG. 19B is a graph correlating the AAF's of indels determined using whole genome sequencing (WGS) and triple-primer PCR sequencing (Trip-Seq). FIG. 19C is a graph showing the correlation of expected and measured AAFs when consistent AAFs are required across multiple unique primer sets. FIG. 19D is a graph of the expected and measured AAFs when triple-primer PCR sequencing is applied to a large set of tissues derived DNA samples for detections of novel mutations in a given gene.

### DETAILED DESCRIPTION OF THE INVENTION

The present disclosure features methods for detecting and quantifying genetic variants in a sample.

The invention is based, at least in part, on the discovery of triple primer PCR sequencing (“TriPP-seq”), which provides a highly sensitive, low-cost approach for detecting and validating mutation on a highly scalable system. Mosaic mutations in somatic or germline cells contribute to a wide range of human disorders. As such, their identification and accurate allelic fraction quantification from tissue-derived and cell-free DNA are essential for clinical diagnoses and early detection of cancers. However, rapid, low-cost detection and validation of ultra-low alternate allelic fraction (AAF) mutations has traditionally required expensive and low throughput methods that have limited widespread testing. Recent methods, (e.g., ddPCR) have shown great promise for detection and validating known mutations at very low AAFs, but remain low-throughput due to allele-specific optimization.

Accordingly, the present disclosure features methods for detecting low frequency genetic variation. The present disclosure's novel approach is based on generating deep coverage of overlapping amplicons of a target nucleic acid sequence. Because the primers used in the reactions are designed to allow discernment and segregation of the overlapping amplicons, the sequencing data can be segregated into groups, and analysis of the sequencing data can be performed in parallel. The methods provide not only deep coverage of the target nucleic acid, but also a cost-effective means of characterizing and validating sequencing results.

Recently, the important roles of somatic mutations beyond cancer are becoming more appreciated with discoveries of somatic mutations across a wide range of neurodevelopmental, overgrowth, and hematological disorders. Even more, the presence of somatic mutations in healthy cells and individuals are associated with normal development and aging and are, therefore, a powerful tool for understanding how cells divide and form complex organs like the human brain. Finally, with the detection of cell-free DNA (e.g., fetal and tumor), it is becoming possible for early detection of disease, tracking of disease recurrence in cancers, and even non-invasive prenatal genetic testing where mutations of the placenta are detected in the pregnant mother's blood sample. The rapid advancements in sequencing technologies and interest in genetic mutation present at low alternate allelic fraction (i.e., ratio of DNA fragments carrying the mutation to those with the wild-type allele in a given samples; AAF) poses some major challenges for both the clinical and research communities related to the sensitivity to detect mutations, false positives, and the precision of the assessed AAFs. These challenges are often confounded by the inability to directly assess tissues with the highest AAFs, as is the case with brain tissue, or by limited or degraded DNA samples, as is typical for cell free DNA.

While germline mutations are relatively easy to detect with small amounts of DNA with variable qualities using WES, WGS, targeted gene panels, and traditional Sanger sequencing due to the equal fractions of mutant to wild-type alleles (50% AAF) in a given DNA sample, the AAF of a somatic mutation will depend on the given tissue, cell type, and the stage in development at which the mutation arose. Traditional WGS and WES sequencing in both the research and clinical diagnostic settings are optimized to identify germline events, but often lack the sequencing depth to robustly detect low-AAF variants. However, many recently improvements allow for robust detection of mutations present at greater than 0.1% AAF. These tools often employ strategies such as molecular barcoding, increased read depth, and reduced use of PCR to mitigate sequencing-induced errors while improving sensitivity. Despite these measures, the identification of somatic alleles, particularly those at very low AAFs has an elevated false positive rate compared to germline mutations. Therefore, while essential, the validation of large numbers of somatic alleles is often challenging due to many factors like assay costs, throughput, and sensitivity limitations.

The methodology utilized to accurately detect or validate somatic mutations have rapidly advanced in the last few years. The challenge for validating or measuring low AAFs is multifaceted, spanning sequencing platforms, inherent error rates of polymerases, and locus specific challenges. Each of these result in additional errors and skewing of AAFs, which can mask or alter the detected AAF in each assay. The utilization of PCR to amplify the genomic loci without inducing additional mutations and maintain the original AAFs has been improved using improved polymerases with proofreading capabilities and, in some cases, unique molecular barcodes for each DNA fragment. Additionally, errors can occur during sequencing on both the Illumina and Ion Torrent platforms. For example, in one study, the Ion Torrent had an error rate ˜0.05% for SNVs but ˜1.5% for indels while the on the Illumina MiSeq had 0.1% errors for SNVs and 0.7% for indels.

The original methods used employed either pyrosequencing or bacterial cloning followed by sanger sequencing of hundreds or thousands of individual bacterial colonies to measure a single mutation. These methods, while accurate and robust, were often cost-prohibitive, less scalable to large numbers of mutations, and were less sensitive for mutations below 5% AAF. These methods were recently succeeded by the advancement of digital droplet PCR, ddPCR, where an allele-specific PCR conditions are designed to allow for the measurement of mutation positive and negative DNA fragments in thousands of droplets. This method is routinely considered a gold standard for validation of somatic alleles in both research and clinical settings, but each allele requires the development of a custom assay, validation and optimization prior to use. The ddPCR assay can accurately detect AAFs below 0.5%, but its sensitivity relies on the quantity and concentration of input DNA and the number of positive droplets formed in each reaction. Despite its great success, the use of ddPCR is somewhat limited as it remains limited by scalability, the potential for allelic dropout, and the ability to design allele-specific primers, which is more challenging in repetitive regions and for small indels.

The growing consensus that somatic mutations might underly a wide range of clinical phenotypes ranging from cancer risk to severe neurodevelopmental and overgrowth conditions suggests that a robust method for both detection and validation of alleles and their mosaic fraction in the body is essential. Here, an improved strategy that aims to mitigate the previously stated limitations for assessing somatic mutations is presented. This strategy, which can be referred to as triple-primer PCR, relies on the power of designing and running at least 3 unique, nonoverlapping amplicons over a suspected mutation. Through independently analyzing each amplicon, the impact of allelic dropout, amplification bias, sequencing and PCR induced artifacts, and general optimization challenges, are markedly reduced while achieving the highest sensitivity to accurately detect ultra-low allelic fractions below 0.1% regardless of tissue origin. As described, below, this triple-primer PCR sequencing method allows for additional improvements to future improve accuracy through incorporations of molecular barcoding and improved purification processes.

### Primers

Nucleic acid amplification according to the presently disclosed methods requires at least two pairs of primers and in some embodiments, at least three pairs of primers. Each pair of primers comprises a forward and a reverse primer, and each primer comprises a complementary nucleic acid sequence that is at least 85% complementary to a nucleic acid sequence (i.e., the primer binding site) on a template nucleic acid molecule. The primers of each pair define the termini of an amplicon that is generated by an amplification reaction, and the region of the amplicon between the termini comprises the target nucleic acid sequence. The combined length of the primers and the target sequence is referred to as the amplicon length. Amplicon length is typically between about 150 and about 500 nucleotides. In some embodiments, the length of the amplicon is about 150, 200, 250, 300, 350, 400, 450, 500, or any integer in-between, nucleotides. In some embodiments, the length of the amplicon is less than 150 nucleotides. In some embodiments, the length of the amplicon is greater than 500 nucleotides. Each primer has a unique nucleic acid sequence that can bind to a complementary primer binding site on the template nucleic acid.

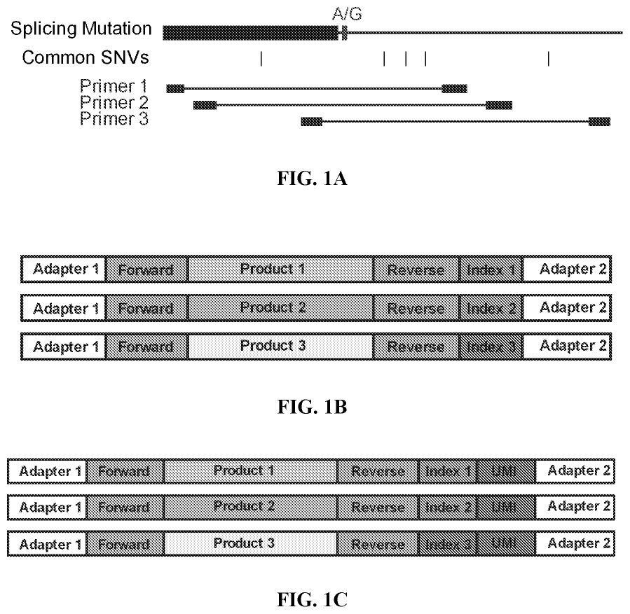

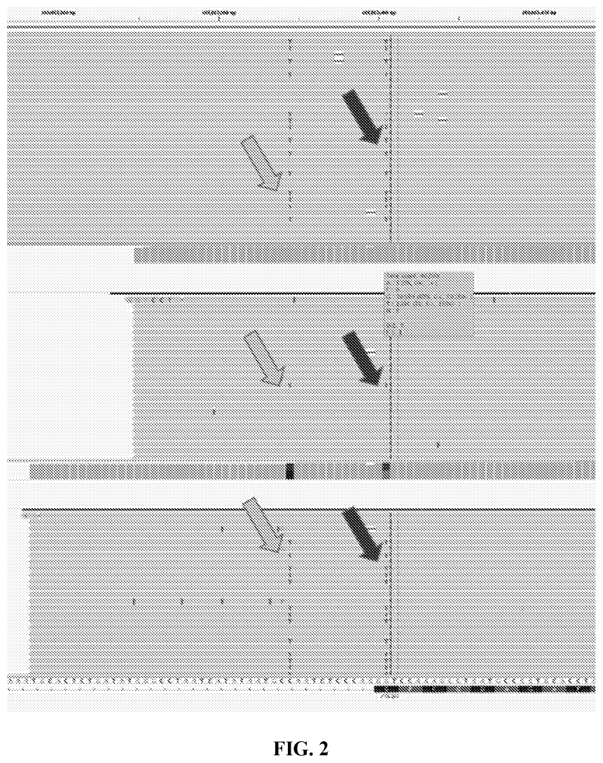

Amplicons generated by amplification reactions using one of the primer pairs will be distinguishable from other amplicons generated by amplification reactions that use different primer pairs due to the length and sequence of the amplicon (FIG. 1A). Each amplicon will include the target nucleic acid sequence, and because the primers are designed to generate overlapping amplicons, each amplicon is at least partially redundant to the other amplicons. In other embodiments, only one primer of each pair will have a unique complementary nucleic acid sequence, such that the amplicons have either the same 5′ terminus nucleic acid sequence and differing 3′ terminus nucleic acid sequences or differing 5′ terminus nucleic acid sequences and the same 3′ terminus nucleic acid sequence.

A primer binding site in a template nucleic acid sequence may harbor a variant that impairs primer biding, which results in decreased amplification of the template harboring the variant and a loss of sequencing coverage of the allele. The resulting loss of coverage of a particular variant is allelic dropout. Referring to FIG. 2, three panels of sequencing data (derived from three sets of overlapping amplicons) show allelic dropout in the middle panel. To minimize allelic dropout in amplification reactions comprising one of three (or more) pairs of primers, at least two forward primers and at least two of the three reverse primers have different complementary nucleic acid sequences. If only two pairs of primers are used, both forward primers and both reverse primers should have unique complementary nucleic acid sequences.

In some embodiments, the complementary nucleic acid sequence of a primer is about 15, 16, 17, 18, 19, 20, 25, 30, 35, or even 40 nucleotides long. In some embodiments, the complementary nucleic acid sequence of a primer is between about 85% and about 100% complementary to a nucleic acid sequence in the template nucleic acid molecule. In some embodiments, the complementary nucleic acid sequence of the primer is between about 85%, 86%, 87%, 88%, 89%, 90%, 91%, 92%, 93%, 94%, 95%, 96%, 97%, 98%, 99%, and 100% complementary to a nucleic acid sequence in the template nucleic acid molecule. In some embodiments, wherein the complementary nucleic acid sequence of the primer is less than 100% complementary with a primer binding site in the template nucleic acid molecule, the mismatch nucleotide or nucleotides in the primer reside at least three bases from the 3′ terminus of the primer. This allows for efficient binding at the terminus of the primer to the template molecule, which facilitates polymerase binding to the primer:template hybrid and extending the primer.

In some embodiments, a primer is comprised of DNA or RNA nucleotides. In some embodiments, a primer comprises at least one modified base. A modified base includes, but is not limited to, those nucleotide analogs described herein or a labeled nucleotide. In some embodiments, a primer may have a modified backbone comprising at least one phosphorothioate linkage. In some embodiments, the primer comprises a label, such as, but not limited to, a fluorescent label, a radiolabel, a nanoparticle label, and/or a biotin label.

In some embodiments, each primer will have an adapter upstream from the complementary nucleic acid sequence. The adapter has a nucleic acid sequence that is complementary to a sequence of a nucleic acid molecule used in a downstream sequencing reaction. For example, the adapters used in some embodiments are designed to be compatible with Next Generation Sequencing including, but not limited to, Ion Torrent and MiSeq platforms. In some embodiments, the length of the adapter is between 8 and 20 nucleotides. In some embodiments, the length of the adapter is 8, 9, 10, 11, 12, 13, 14, 15, 16, 17, 18, 19, or 20 nucleotides. The adapter's sequence is designed to reduce or eliminate nonspecific binding of the adapter to the template nucleic acid molecule. In some embodiments, the adapter is designed to have a sequence that is not substantially complementary to any nucleic acid sequence present in the template nucleic acid molecule. In some embodiments, the adapter is designed to diverge from perfect complementarity with the template by 2, 3, or 4 or more nucleotides.

At least one primer in each pair also has an index sequence, or barcode (FIG. 1B). The index sequence allows for rapid identification of sequencing data generated from similar amplicons. The index sequence as contemplated herein can be between 8 and 30 nucleotides in length. For example, the index sequence contemplated herein may comprise 8, 9, 10, 11, 12, 13, 14, 15, 16, 17, 18, 19, 20, 21, 22, 23, 24, 25, 26, 27, 28, 29, or 30 nucleotides. Similar to the adapter, the index sequence is designed to reduce or eliminate nonspecific binding of it to the template nucleic acid molecule. In some embodiments, the index sequence comprises a nucleic acid sequence that is not substantially complementary to any nucleic acid sequence present in the template nucleic acid molecule. In some embodiments, the index sequence is designed to diverge from perfect complementarity with a nucleic acid sequence in the template nucleic acid molecule by 2, 3, or 4 or more nucleotides. In some embodiments, the index sequence is designed so that the most complementary sequence in the template has a conformation or structure that disfavors index sequence binding.

In some embodiments, at least one primer in each pair comprises a unique molecular identifier (UMI) (FIG. 1C). A UMI may allow for the detection of redundant sequencing reads. As contemplated herein, the UMI will comprise between 5 and 20 nucleotides. For example, the UMI contemplated herein may comprise 5, 6, 7, 8, 9, 10, 11, 12, 13, 14, 15, 16, 17, 18, 19, or 20 nucleotides. In some embodiments, no two primers will have the same UMI. Similar to the adapter and the index sequence, UMIs are designed to reduce or eliminate nonspecific binding of the UMIs to the template nucleic acid molecule. In some embodiments, the UMI comprises a nucleic acid sequence that is not substantially complementary to any nucleic acid sequence present in the template nucleic acid molecule. In some embodiments, the UMI is designed to diverge from perfect complementarity with the template by 2, 3, or 4 or more nucleotides. In some embodiments, the UMIs are designed so that the most complementary sequences in the template nucleic acid have a conformation that disfavors UMI binding.

There are approximately 1,000 possible sequences for a 5-nucleotide UMI, approximately 65,000 possible sequences for an 8-nucleotide UMI, approximately 1×10⁶ possibilities for a 10-nucleotide UMI, and approximately 1×10¹² possibilities for a 20-nucleotide UMI. Even if some UMIs are not suitable for the reasons given above, large UMI libraries can be produced for use in the presently disclosed methods. Use of nucleotide analogs increases the number of possible sequences for a UMI.

Table 1 characterizes five primer pairs used in the disclosed methods. In this table, “Chr. No.” means chromosome number; “Ref” refers to the reference nucleotide; and “Alt” refers to the alternate nucleotide. Each of the primer pairs is designed to amplify a region containing a single nucleotide variant (the “allele start” and “allele end” are the same locus number). Three of the primer pairs on Table 1 (X:153579431-153579431/T/C-F1; X:153579431-153579431/T/C-F2; and X:153579431-153579431/T/C-F3) are used to interrogate a single nucleotide variant in the Filamin A (FLNA) gene on the X chromosome. The remaining two primer pairs (X:153579431-153579431/T/C-F1 and X:153579431-153579431/T/C-F2) are used to interrogate a single nucleotide variant in the SR-Related CTD Associated Factor 11 (SCAF-11) gene on chromosome 12. The amplicons generated in amplification reactions comprising the primer pairs disclosed in Table 1 will be about 220 to 260 nucleotides in length.

TABLE 1

| | | Chr. | Allele | Allele | | | | Sample | Prod. | Prod. | Insert | Insert |
|---|---|---|---|---|---|---|---|---|---|---|---|---|
| | PrimerID | No. | Start | End | Ref | Alt | Gene | ID | Start | End | Start | End |
| 1 | X:153579431- | X | 15357 | 15357 | T | C | FLNA | PH4201 | 153579266 | 153579517 | 153579284 | 153579499 |
| | 153579431/T/ | | 9431 | 9431 | | | | | | | | |
| | C-F1 | | | | | | | | | | | |
| 2 | X:153579431- | X | 15357 | 15357 | T | C | FLNA | PH4201 | 153579289 | 153579555 | 153579311 | 153579536 |
| | 153579431/T/ | | 9431 | 9431 | | | | | | | | |
| | C-F2 | | | | | | | | | | | |
| 3 | X:153579431- | X | 15357 | 15357 | T | C | FLNA | PH4201 | 153579379 | 153579637 | 153579397 | 153579619 |
| | 153579431/T/ | | 9431 | 9431 | | | | | | | | |
| | C-F3 | | | | | | | | | | | |
| 4 | 12:46321441- | 12 | 46321 | 46321 | T | G | SCAF11 | PH4201 | 46321317 | 46321542 | 46321343 | 46321517 |
| | 46321441/T/ | | 441 | 441 | | | | | | | | |
| | G-F1 | | | | | | | | | | | |
| 5 | 12:46321441- | 12 | 46321 | 46321 | T | G | SCAF11 | PH4201 | 46321246 | 46321470 | 46321271 | 46321448 |
| | 46321441/T/ | | 441 | 441 | | | | | | | | |
| | G-F2 | | | | | | | | | | | |

| | | | | Barcode | Primer | | | |
|---|---|---|---|---|---|---|---|---|
| | Primer ID | Forward | Reverse | No. | barcode | type | Forward | UMI |
| 1 | X:153579431- | CAGGGCCTCACC | ttaacggacgCGCCAGAT | ttaacggacgC | 1 | Bar- | CAAGGT | No |
| | 153579431/T/ | TTGGTC | GGGTAAGTGC | GCCA | | code | GAGGCC | |
| | C-F1 | | | | | | CTG | |
| 2 | X:153579431- | CTGTGACATAGC | tccggcttacTGCAAATC | tccggcttacT | 2 | Bar- | AGTGCT | No |
| | 153579431/T/ | ACTCCTCCAG | AGTGGCTCTCC | GCAA | | code | ATGTCAC | |
| | C-F2 | | | | | | AG | |
| 3 | X:153579431- | AGGCTGGCTGGT | tctcattcagCTCCCTTCC | tctcattcagC | 3 | Bar- | TCAACC | No |
| | 153579431/T/ | TGACCT | TGCCACCTG | TCCC | | code | AGCCAG | |
| | C-F3 | | | | | | CCT | |
| 4 | 12:46321441- | AATCACACTCCA | geggtcatacACATGTGA | gcggtcatacA | 1 | Bar- | CTATGG | No |
| | 46321441/T/ | TAGGTATCATTTC | TACTTTTGGGAATG | CATG | | code | AGTGTG | |
| | G-F1 | A | AAG | | | | ATT | |
| 5 | 12:46321441- | TTCATTCATTTGT | taggacgttcCTTCTGAA | taggacgttcC | 2 | Bar- | AAACAA | No |
| | 46321441/T/ | TTAAGATCAGCA | CACCAAATTGGAAA | TTCT | | code | ATGAAT | |
| | G-F2 | | | | | | GAA | |

### Template Nucleic Acid

Samples comprising template nucleic acid molecules to be assayed using the methods disclosed herein can be obtained from a variety of sources including, but not limited to, tissue biopsies, blood draws, buccal swabs, hair, sweat, skin, semen, and mucus. In some embodiments, the sample comprises cells from a subject, for example, circulating tumor cells, blood cells, skin cells, and the like. In some embodiments, the sample comprises cell free nucleic acid, such as, but not limited to, cell free tumor nucleic acid and cell free fetal nucleic acid. In some embodiments, the template nucleic acid molecule is isolated or purified before amplification. Methods of isolating and purifying nucleic acids are well known in the art. Template nucleic acid molecules comprise at least one target nucleic acid sequence. The target sequence is flanked by primer binding sites. In some embodiments, the template is a DNA molecule. In some embodiments, the template is an RNA molecule. In some embodiments, the template may be double-stranded, while in other embodiments, the template is single-stranded.

In some embodiments, the target nucleic acid is a portion of a gene such as, but not limited to, ABCC8, ABLIM3, ACBD3, ACIN1, ACSL5, ACTA2, ACVR1, ACVR1B, ACVR1C, ACVR2B, ADAMTSL3, ADORA2A, AEBP2, AES, AFAP1, AGAP1, AKR7A2, AKT1, ALK, AMHR2, AMPD3, ANGPTL6, ANO7, APC, APOL2, AQP4-AS1, ARHGEF3, ARID1A, ARIDSA, ARIH1, ARNT, ATM, ATP5A1, ATP9B, ATXN7L1, AX747372, BAG1, BAIAP2L1, BECN2, BMP4, BMP8A, BMP8B, BMPR1A, BMPR1B, C12orf60, C17orf89, C1ORF210, C6ORF10, C6orf211, C9orf40, CACNA1A, CACNA1H, CACNA2D4, CAMK1D, CAMKMT, CARM1, CAST, CBS, CCBE1, CDC40, CDH23, CDH4, CDKN2B, CHRNA4, CLASP1, CLCA1, CLDN2, CLIC3, CNN3, CNTN1, COL11A2, COL3A1, COL3A2, COL4A1, COL4A5, COL4A6, COL5A1, COL5A2, COL6A2, COL6A3, COX7A2L, CRADD, CREBBP, CRY2, CSGALNACT2, CTBP2, CYP2S1, DAG1, DCAF8, DCAF8,DCAF8, DLAT, DLGS, DLGAP4-AS1, DNAH3, DOCK4, DOCK8, DOPEY1, DPYSLS, DYNC1H1, DYNC1I2, DYRK2, E2F4, E2F6, ECI2, EEF1DP3, EHD4, EIF2B5, EIF4G3, ELAC2, ELK3, EMD, EMX20S, EPPK1, EPT1, ERBB4, ERCCS, ETS2, ETV4, FAM107B, FAM13B, FAM175A, FAM83E, FAV, FBN1, FBN2, FBN3, FBXO28, FGFR2, FHL2, FIRRE, FLNA, FLT3, FOXA3, FOXG1-AS1, FST, GABRG1, GALM, GAPDH, GDF6, GDF7, GLI2, GLI3, GLRXS, GLT8D2, GOLPH3, GPD2, GPR68, GPRASP1, H2AFX, HDAC4, HHAT, HIST1H2AH, HIST2H2AB, HK1, HMCN1, HMSD, HNF4A, HNRNPU, HOXD3, HPS3, HS3ST3A1, IDH1, IFNG, IKBKAP, IMP3, INHBA, INPP4B, INPP5A, IQCK, JAG1, JWT213-1, JWT213-2, JWT213-3, JWT213-4, JWT213-5, JWT213-6, JWT213-7, JWT213-8, JWT213-9, JWT307_1, JWT307_2, JWT307_3, JWT307_4, JWT307_5, JWT307_6, JWT307_7, JWT310-1, JWT310-2, JWT310-3, JWT310-4, JWT310-5, JWT310-6, JWT310-7, JWT311-1, JWT311-2, JWT311-3, JWT311-4, JWT311-5, JWT311-6, JWT311-7, JWT312-1, JWT312-2, JWT312-3, JWT312-4, JWT312-5, JWT312-6, JWT312-7, JWT312-8, JWT312-9, JWT313-1, JWT313-2, JWT313-3, JWT313-4, JWT313-5, JWT313-6, JWT313-7, JWT313-8, JWT313-9, JWT364_1, JWT364_2, JWT364_3, JWT364_4, JWT364_5, JWT364_6, JWT364_7, KANSL1, KCNQ1, KDM3A, KDR, KIRREL3, KLF13, KLHL14, KMTD2, L3MBTL1, LACTB2, LAMA2, LAMA3, LEFTY1, LINGO4, LMAN2L, LRRC4C, LSAMP, LTBP1, LTBP2, LTBP3, LZTS2, MAD1L1, MAD2L1, MAEA, MAGI2, MAML2, MAP3K7, MAPK1, MAPK3, MAPK8IP2, MARK3, MAT2A, MATR3, MBNL2, MCL1, MCU, MECP2, MED12, MED29, MEF2A, MEGF6, MESD, METTL17, MIER2, MIR181A1HG, MKL1, MKL2, MLH1, MOB2, MPRIP, MRPL32, MRS2, MTCH1, MTOR, MUC16, MUC3A, MYC, MYH11, MYH11,NDE1, MYH11; MYH11, MYLK, MYLK-AS1, MYOCD, NA, NDFIP2, NDUFC1, NEK9, NF1, NFKB1, NGEF, NME4, NME4,DECR2, NOL9, NOTCH1, NOTCH3, NPLOC4, NRG4, NRM, NRTN, NTM, NUCB1, NUDT16, NUDT16L1, OAS3, OR4K3, OSTC, PAG1, PCDH15, PDCD6, PDE4DIP, PDSSA, PHC1, PHF12, PHKG1, PIK3R1, PLEKHG6, PLXDC2, PMM2, POLG2, POLR3B, PPARGC1A, PPHLN1, PPP1R14A, PPP1R15B, PRAF2, PRDM16, PRKG1, PRPH2, PRTG, PTGDR, PTPN12, PTPN14, PTPRC, PTPRS, PUS7, RABL6, RALGAPA1, RAPGEF4, RBM10, REPS2, RHBDF2, RIN2, RNF175, RNU1-35P, RNU1-35P, RP11-149P24.1, ROCK1, ROCK2, RPRD2, RSF1, RUSC1, SAFB2, SASH1, SCAF11, SCARF1, SEPT11, SH3GLB2, SHPK, SHPK, SHPK, SHROOM3, SIKE1, SIPA1L1, SIRPA, SK213, SK215, SLAIN1, SLC1A4, SLC25A48, SLC2A10, SLC4A1AP, SLMO2, SLTM, SLX4, SMAD3, SMAD4, SMAD5, SMAD6, SMAD7, SMARCA4, SMLR1, SMTNL1, SMURF1, SNK307, SNK310, SNK311, SNK312, SNK313, SNK364, SNK380, SNK382, SNK383, SNK384, SNK385, SNK386, SOX21-AS1, SOX9, SPOCK2, SPRED1, SPSB2, SRGN, SRP68, SRRM2-AS1, ST6GAL1, STK16, STRN3, SUCLA2, SUCO, SWI5, SYNE2, TAB1, TBC1D13, TBCE, TCERG1, TCF4, TERT, TFB2M, TFDP1, TGFB1, TGFB3, TGFBR1, TGFBR2, THBS1, TMEFF2, TMEM132C, TMEM2, TMEM268, TNPO1, TPCN2, TPM3, TPRX1, TRAM1, TRAPPC9, TRPM1, TSC2, TSHZ2, TTN, TUBG1, TUBGCP3, TULP4, UBAP2, UBE2I, UBE2W, UHRF1, UNC45A, UNG, UROC1, USP24, USP34, USP8, VANGL1, VIPR2, VPS13D, WDR35, WDR45B, WDR77, WDSUB1, WHSC1, YARS2, YIPF3, ZFHX4, ZFYVE16, ZFYVE9, ZMIZ1, ZNF223, ZNF292, ZNF3, ZNF362, ZNF451, ZNF517, ZNF593, ZNF630, ZNRF3, or ZSCAN5A.

The subject from whom the template nucleic acid molecule sample is obtained can be any organism. In some embodiments, the subject is a vertebrate. In some embodiments, the subject is a mammal such as a human, mouse, rat, dog, cat, horse, cow, sheep, or other domesticated mammal. In some embodiments, the mammal is a human. In some embodiments, the subject from whom the sample is obtained has or is suspected of having a disease or condition associated at least in part with a genetic variant or variants.

### Polymerases

The methods provided herein use a nucleic acid polymerase to amplify a target nucleic acid sequence. Because some polymerases have high error rates (incorporating the wrong nucleotide at a position in a synthesized nucleic acid), selection of a suitable polymerase is an important concern. Sequence errors introduced by a polymerase confound authentic sequence data, making discernment of low frequency variants unreliable or expensive due to the amount of coverage necessary to overcome the polymerase's error rate. High-fidelity polymerases, are particularly well-suited for use in the presently disclosed methods, and can be used to synthesize copies of a target nucleic acid sequence that potentially harbors a low-frequency variant. Such high-fidelity polymerases introduce fewer nucleotide sequence errors than non-high-fidelity polymerases. Thus, in some embodiments, the nucleic acid amplification reactions comprise a high-fidelity nucleic acid polymerase. For example, in some embodiments, nucleic acid reactions comprise a Phusion high-fidelity DNA polymerase (New England Biolabs (NEB)). This polymerase has a reported error rate of 4.4×10⁻⁷ errors per base in Phusion HF buffer and 9.5×10⁻⁷ errors per base in GC buffer. Thermus aquaticus (Taq) polymerase has a 50-fold higher error rate than the error rate of the Phusion high-fidelity polymerase. Other polymerases may be used to amplify nucleic acids according to the presently disclosed methods, but an increase in polymerase error rates may decrease the reliability of the method. Table 2 provides a summary of the differences between the high-fidelity Phusion DNA polymerase and the Pyrococcus furiosus and the Taq DNA polymerases (HF=high-fidelity; “GC Buffer” refers to a buffer suited for reactions amplifying a target rich in G and/or C). To overcome such errors generated by non-high-fidelity polymerases, additional coverage of the interrogated nucleic acid may be necessary, resulting in increased costs.

TABLE 2
Polymerase Comparison

| Polymerase | 1 kb Template | 3 kb Template |
|---|---|---|
| Phusion High-Fidelity DNA Polymerases | 1.32% | 3.96% |
| (HF Buffer) | | |
| Phusion High-Fidelity DNA Polymerases | 2.85% | 8.55% |
| (GC Buffer) | | |
| Pyrococcus furiosus DNA polymerase | 8.4% | 25.2% |
| Taq DNA polymerase | 68.4% | >200% |

### Overview of the Method

The methods disclosed herein are suitable for detecting low frequency variants. The methods described herein involve detecting the presence or absence of low frequency genetic variation in a nucleic acid molecule by amplifying the nucleic acid sequence of interest using multiple pairs of primers. Each pair of primers comprises a forward primer and a reverse primer, each having a unique binding sequence complementary to a target polynucleotide, wherein the intervening sequences between each pair of primers (i.e., the amplified nucleic acid sequence) at least partially overlap. The resulting overlapping amplicons are sequenced using a Next Generation Sequencing platform, which provides the deep coverage necessary to validate low frequency variants. The sequencing reads are aligned, and determinations regarding the presence or absence of genetic variation are made. The sequencing data can be used for further characterization of any detected genetic variation (i.e., alternative allele fraction).

In some embodiments, the low frequency variant is a known variant, and the methods disclosed herein may be used to confirm the variant's presence and/or characteristics (i.e., its alternate allele frequency). In some embodiments, the low frequency variant originated during a germline event, while in other embodiments, the low frequency variant to be interrogated originated during a somatic event. In some embodiments, the low frequency variant is a silent variant, a missense variant, or a nonsense variant. In some embodiments, the low frequency variant alters a splice site or is an insertion or deletion.

### Amplification

In some embodiments, nucleic acid amplification reactions comprise a template nucleic acid molecule having a target nucleic acid sequence, at least three primer pairs suitable for interrogating the target nucleic acid, nucleotides, and a polymerase. Due to the use of at least three primer pairs in the amplification, the overall method described herein can be referred to a triple-primer PCR sequencing. In some embodiments of the present disclosure, the reaction further comprises a buffer that provides a suitable ionic environment for the polymerase to synthesize a nucleic acid molecule. In some embodiments, the reaction comprises a buffer having essential cofactors (e.g., magnesium) necessary for polymerase function. In some embodiments, the cofactors necessary for proper polymerase function are added to the reaction independently of the buffer.

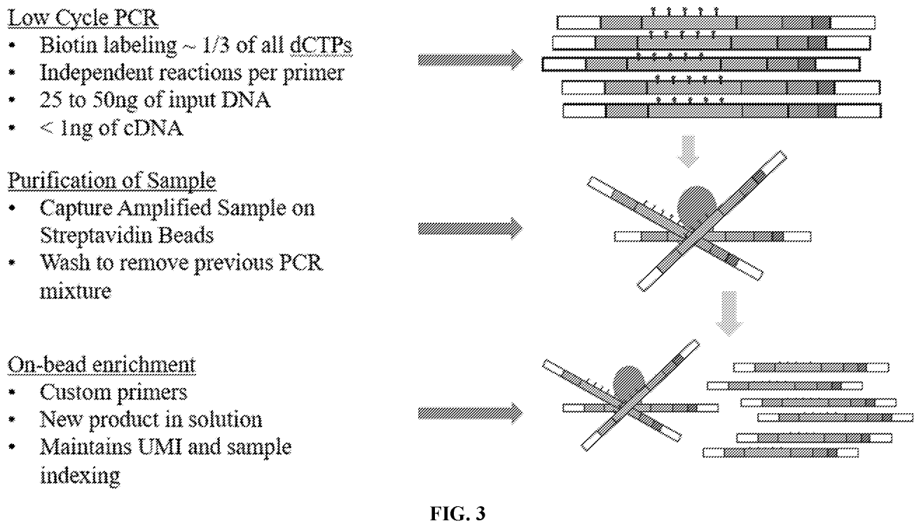

In some embodiments, the amplification reaction comprises labeled nucleotides, wherein the labeled nucleotides facilitate efficient capture of any amplicon that comprises one or more labeled nucleotides. Referring to FIG. 3, a nucleotide may be labeled with biotin, and amplicons incorporating the biotin-labeled nucleotides can be captured on streptavidin beads or other media or substrate comprising streptavidin. These captured amplicons can be used as templates for a subsequent amplification reaction, thereby enriching the captured amplicons.

In some embodiments, separate nucleic acid amplification reactions are prepared for each pair of primers. For example, amplifying a target nucleic acid sequence may comprise at least three reactions according to the methods described herein, wherein each reaction comprises one of three different pairs of primers. The primers, as discussed supra, are used in amplification reactions that generate overlapping amplicons (i.e., semi-redundant interrogation of the target nucleic acid sequence), thereby reducing the probability of impaired detection of variants or skewed downstream determination of alternate allele fractions due to amplification bias. In some embodiments, a single amplification reaction will comprise all pairs of primers. Combining the different primers into a single amplification reaction will generate a greater number of distinct amplicons.

In some embodiments, the amplification reactions are polymerase chain reactions (PCR). PCR reactions undergo multiple thermocycles, wherein each thermocycle comprises a denaturing step, an annealing step, and an extension step. During the denaturation step, the reaction is incubated at or above 90° C., which is a sufficient temperature, in some embodiments, to cause a double-stranded DNA molecule to denature into single DNA strands or to cause the nucleic acid molecule to undergo a conformational change that is more conducive for an amplification reaction.

The annealing step comprises complementary binding of the primers to the template nucleic acid and occurs at a lower temperature than that used in the denaturing step. In some embodiments, each primer will be designed to anneal to a complementary nucleic acid sequence at a temperature of between about 50° C. and about 65° C. In some embodiments, the annealing temperature is about 50° C., 51° C., 52° C., 53° C., 54° C., 55° C., 56° C., 57° C., 58° C., 59° C., 60° C., 61° C., 62° C., 63° C., 64° C., or 65° C. about 50° C., 51° C., 52° C., 53° C., 54° C., 55° C., 56° C., 57° C., 58° C., 59° C., 60° C., 61° C., 62° C., 63° C., 64° C., or 65° C. In some embodiments, the temperature at which the primers anneal to the nucleic acid template can be modified by adjusting conditions (e.g., salt concentration) in the sample or in the amplification reaction. One skilled in the art will understand how changing sample or reaction conditions can affect the temperature at which a primer binds to template nucleic acid.

In the extension step of a PCR cycle, the primers annealed to the template nucleic acid's primer binding sites are extended by a polymerase to produce a nucleic acid molecule that is complementary to a portion of the template nucleic acid molecule. A proper extension temperature is at or about the optimal temperature for the polymerase to synthesize a nucleic acid molecule. In some embodiments, the extension temperature is between about 65° C. and 75° C. In some embodiments, the extension temperature is about 65° C., 66° C., 67° C., 68° C., 69° C., 70° C., 71° C., 72° C., 73° C., 74° C., or 75° C. In some embodiments, the extension temperature may be 5, 10, 15, 20, or 25% higher or lower than the optimal temperature of the polymerase. Those skilled in the art will understand how to adjust the temperatures, or other reaction conditions, necessary for successful PCR amplification of a nucleic acid sequence.

In some embodiments, the template nucleic acid is amplified isothermally. For example, helicase dependent amplification is an isothermal amplification method that utilizes a helicase, rather than high temperatures, to separate the strands of a duplex nucleic acid. By not requiring a denaturation step, the isothermal reaction can be incubated at or about the optimal temperature of the polymerase. However, in some embodiments, the isothermal amplification reaction comprises an initial heat denaturation step. Exponential amplification is achieved by incubating the reaction at an isothermal temperature, which obviates the need for thermocycling equipment. Other isothermal amplification techniques are known in the art, and one skilled in the art would understand how to optimize these techniques to comport with the methods described herein.

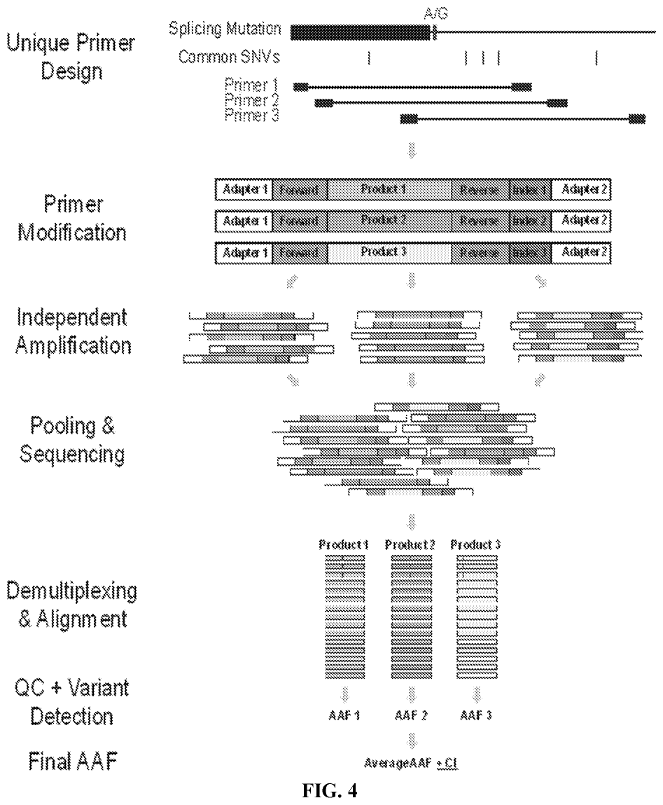

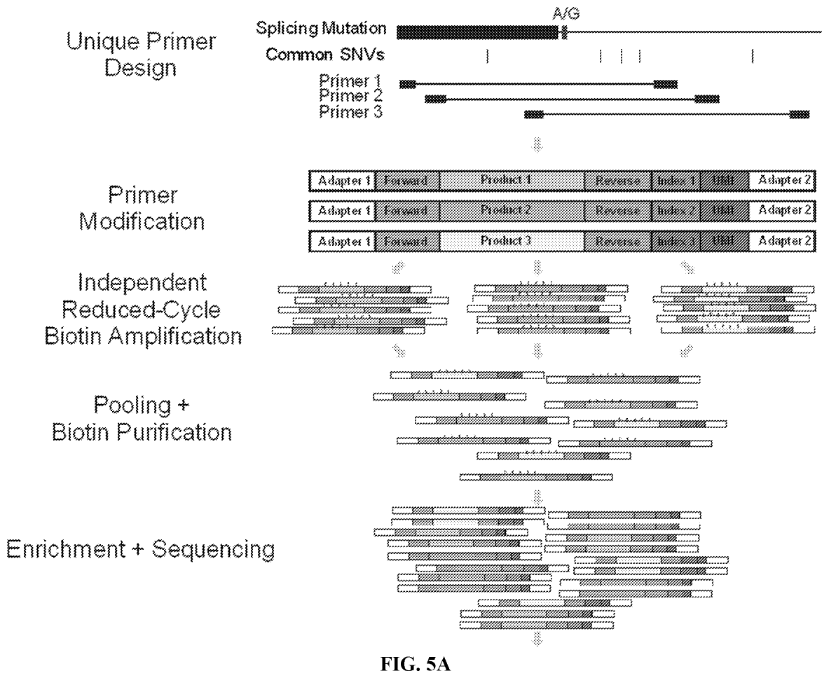

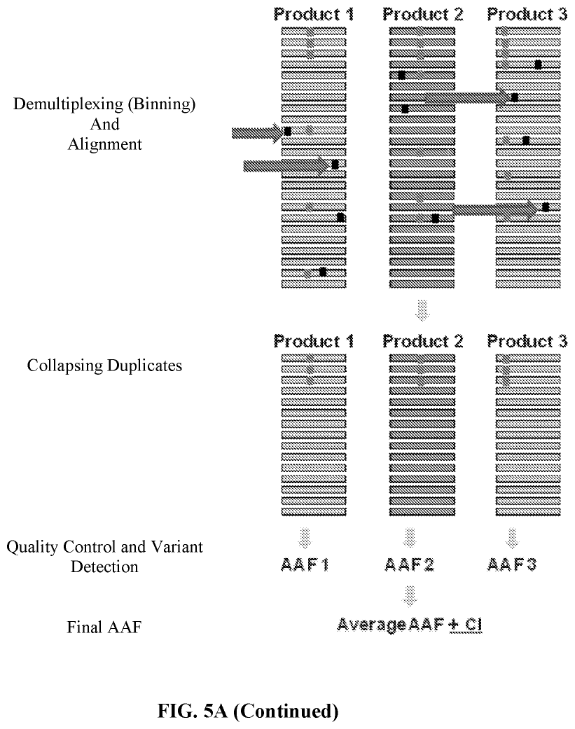

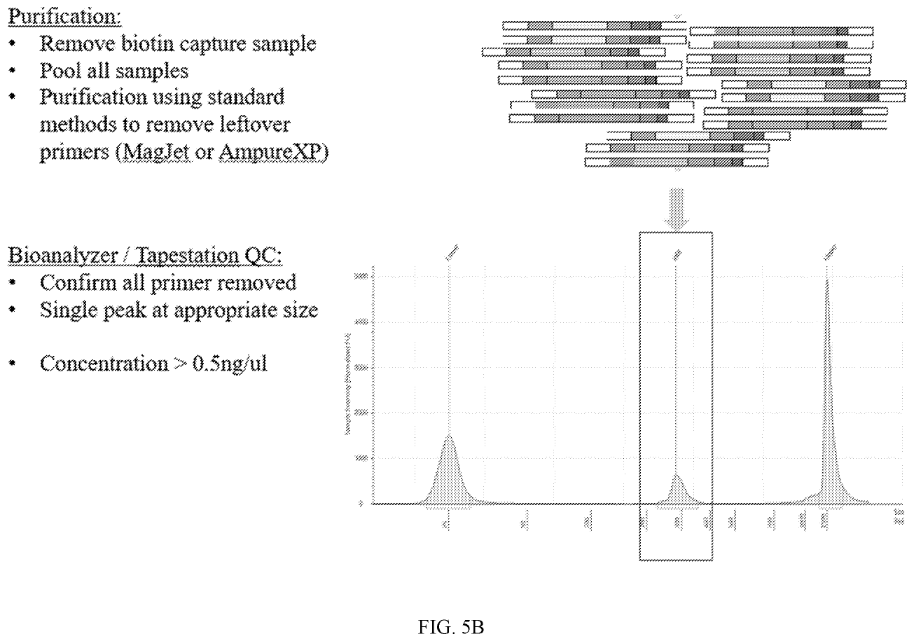

Referring to FIGS. 4 and 5, in some embodiments, the amplification reaction products (amplicons) are pooled. This allows simultaneous sequencing of the amplicons generated by the different amplification reactions, which decreases reagent costs and the burden on laboratory personnel and equipment. In some embodiments, the amplification reactions are not pooled prior to sequencing. Pooling, in some embodiments, comprises combining all the amplicons, while in some embodiments, pooling of only a subset of the amplification reactions is required. Additionally, in some embodiments, only a portion of each amplification reaction is pooled, and the remaining unpooled amplification reactions are assayed in parallel with different techniques.

In some embodiments, the amplification reaction products are purified or isolated before pooling. Methods for isolating and purifying nucleic acids are well known in the art, and there are many commercially available kits for purifying or isolating amplicons. In some embodiments, purifying or isolating amplicons occurs after pooling. In some embodiments, enriched amplicons resulting from biotin:streptavidin capture and reamplification, can be purified using streptavidin to bind and separate all biotin labeled amplicons.

In some embodiments, the amplicons are assessed prior to being sequenced. Assessing the amplicons can include, for example, gel electrophoresis, real time detection, or spectrophotometric determination of amplicon concentration. For example, amplicons may be assessed using a TapeStation (Agilent) or Bioanalyzer 2100 (Agilent). These analyses allow an investigator to determine if the amplification reaction generated sufficient amounts of high quality amplicons for subsequent sequencing.

### Sequencing

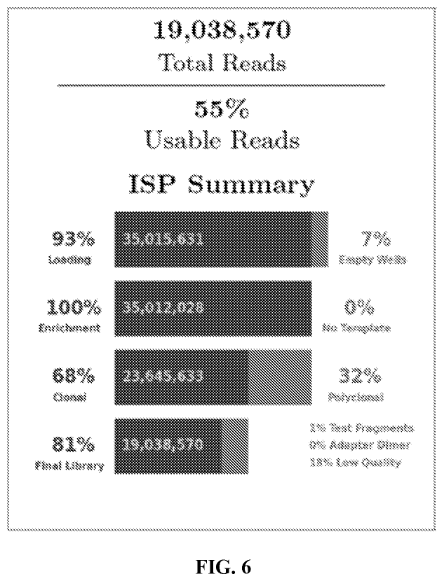

Sequencing of the overlapping amplicons provides multiple independent interrogations of a variant nucleotide or nucleic acid sequence compared to using a single pair of primers. Traditional Sanger sequencing platforms can be used to sequence the overlapping amplicons, but this approach is inefficient for detecting rare variants. Conversely, Next Generation Sequencing (NGS) platforms can generally accommodate thousands of sequencing reactions run in parallel, thereby providing deeper coverage than is possible with Sanger sequencing. For example, referring to FIG. 6, the Ion Torrent system can generate nearly twenty million reads with 93% ion sphere particle (ISP) loading. Ion sphere particles used in the Ion Torrent system are conjugated directly or indirectly to a nucleic acid comprising the sequence of interest adjacent to a nucleic acid sequence complementary to the adapter described supra. In detecting, characterizing, or validating low frequency variants, this increased coverage enables distinguishing true variants from errors introduced during amplification, sequencing, or data processing.

The amplicons to be sequenced are, by design, generally less than 300 nucleotides in length, and there are several NGS platforms that can cost-effectively generate sequencing data at the desired coverage level. For example, ThermoFisher's Ion Torrent and Illumina's MiSeq can each generate maximum read lengths of approximately 250 nucleotides. Other NGS approaches are available for shorter or longer read lengths. For example, Illumina's HiSeq platform has a maximum read length of about 150 nucleotides, while the Roche 454 platform can generate at least 400 nucleotide reads. One skilled in the art will be to determine which platform can be used to generate the desired sequencing data, and will optimize the adapters on each primer to comport with that platform.

### Data Processing and Analysis

In some embodiments, the sequencing data is assessed for quality before alignment, and those reads not possessing the required quality characteristics are removed from the data set. Typically, quality control of sequencing reactions comprises establishing a signal-to-noise threshold, and reads that do not meet the threshold are discarded. Such quality control lessens the probability of erroneous base calls in a read that would decrease reliability of the assay.

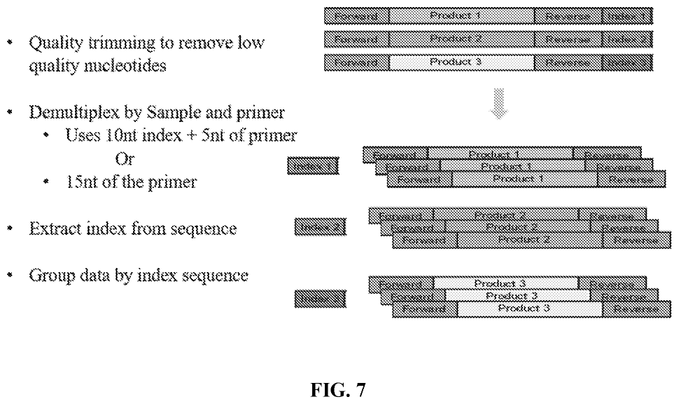

Sequencing data generated using the disclosed methods can be processed to accurately determine alternate allele frequencies. Referring to FIG. 7, in some embodiments, the sequencing data is first demultiplexed by grouping together all reads having the same index sequence. Each pair of primers used to amplify a target nucleic acid sequence has a unique index sequence, such that data generated for the products of distinct amplification reactions will be segregated into distinct bins based on their index sequence. All sequences having the same index sequence will be binned together and segregated from sequences having different index sequences. This demultiplexing of the sequencing data allows for three independent determinations of the alternate allele fraction for variants detected in the target nucleic acid sequence and the assignments of confidence intervals. In some embodiments, the average alternate allele fraction is determined by averaging the three individual alternate allele fractions.

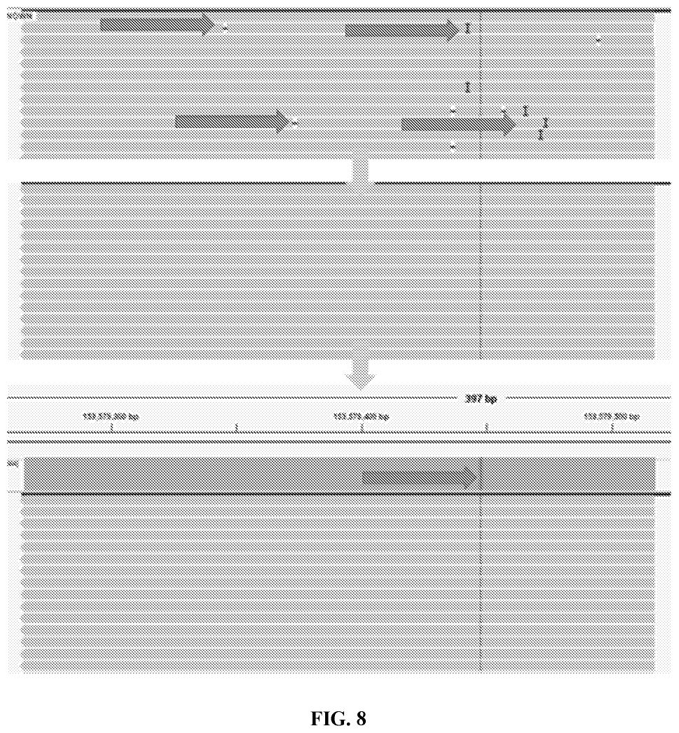

The data in each bin is aligned to provide maximal sequence identity between the individual reads. For example, if a read has a single nucleotide deletion, the alignment will incorporate the deletion into the read's aligned sequence so that the nucleotide sequences on either side of the deletion align with other reads that do not have the deletion. Referring to FIG. 8, indels are elevated in Ion Torrent sequencing, and these errors can mask true alleles (especially low frequency variants) (top panel). However, the Pullox Algorithm can identify and correct about 97% of such indel errors and does not impact mosaic alleles (middle panel). This program can also reduce background noise up to 50%. The processed data can be mapped to the genome or template nucleic acid and is able to identify the target allele (bottom panel).

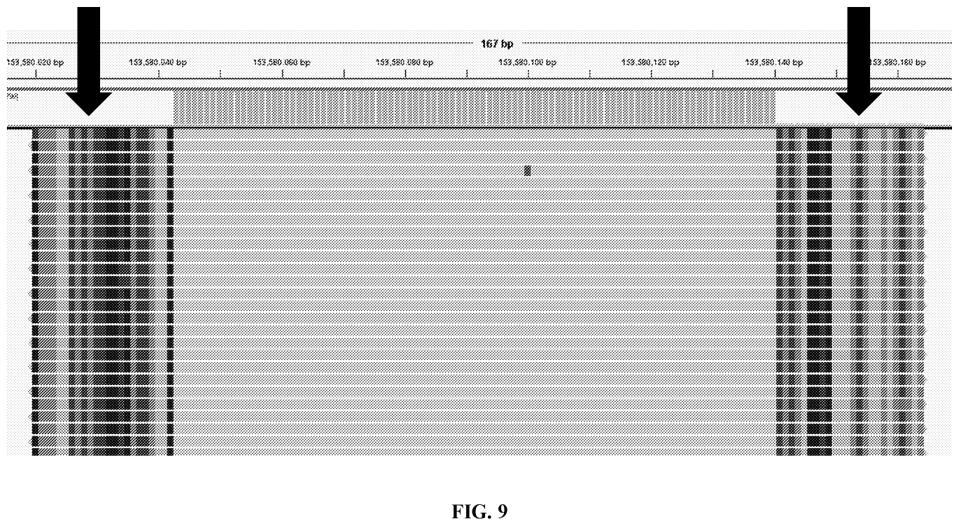

Primer binding sites are also identified (FIG. 9) and removed from the sequencing data. Because these sequences are known, they can be readily identified and removed, which avoids analyzing possible false positive and false negative results in these sequences.

In some embodiments, all but one read having the same unique molecular identifiers will be removed from the data set, which indicates multiple amplification reactions that used the exact same primer. These duplicated amplifications reactions are not considered independent interrogations of the nucleic acid. Retention of such redundant data could impact alternate allele fraction determination. In some embodiments, accurate determination or validation of alternate allele frequencies of about 0.025% comprise removing redundant reads from the data. In some embodiments, wherein the alternate allele fraction is known to be 0.1% or greater, removal of redundant reads may not be necessary due to the deep coverage available in Next Generation Sequencing platforms. Once the alignment is set in each bin, the alternate allele frequencies for variants in each bin are determined.

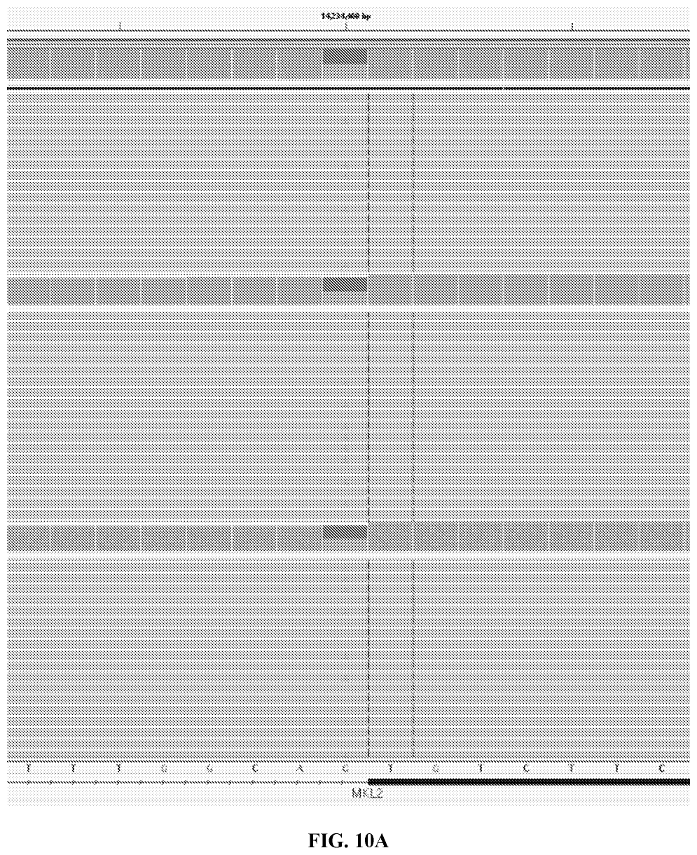

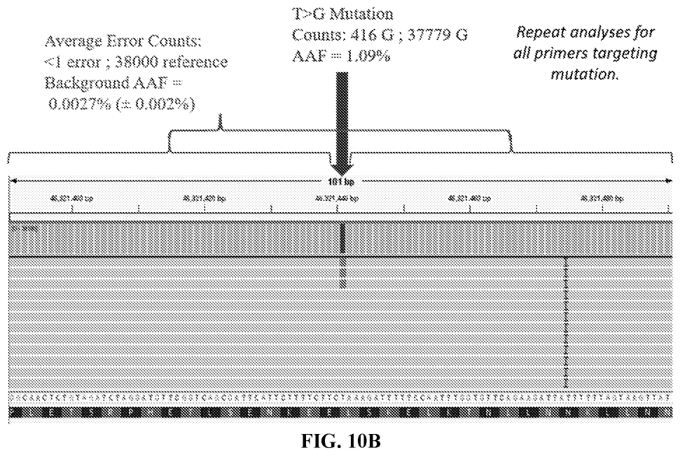

The methods provided can distinguish between germline and somatic events resulting in genetic variation. Referring to FIG. 10A, a genetic variant derived from a germline event, which should approach an alternative allele frequency of about 50%, is shown. Three panels of sequencing data are separated by the large shaded boxes, wherein each panel presents a subset of sequencing data for amplicons generated from different amplificant reactions. In each panel, the allele frequency is nearly identical in each panel (Panel 1: 49.5% (112,000× coverage); Panel 2: 49.9% (75,000× coverage); and Panel 3: 50.0% (126,000× coverage). The alternate allele frequencies are then averaged for each variant and a confidence interval assigned. Those skilled in the art will understand how the frequencies are determined and will know that commercially available algorithms can be employed.

A somatic event occurring in a single subject will likely have a much lower allele frequency than an inherited allele, and a subject having a genetic variant derived from a somatic event is said to be mosaic for the variant. As shown in Table 3, the alternate allele frequencies (AAF) observed in three different amplicon samples are about 1%, well below the frequency expected in an individual for an inherited allele, which suggests the variant is a somatic mosaic variant. For example, for the sequencing reads of amplicons generated using the Primer 1 set of primers, 416 reads out of 37,779 total reads contained the alternate allele (FIG. 10B). The “Background AAF” is the alternative allele frequency of variants detected in the regions flanking the alternate allele (also referred to as the “background rate”). In some embodiments, sequencing data of the primer binding sites is removed prior to determining a background rate. This improves the accuracy of the background rate because sequencing errors are more prevalent for regions near the adapter binding sites (e.g., primer binding sites).

TABLE 3
Alternative Allele Fractions

| Primer #: | Allele Counts | AAF | Background AAF |
|---|---|---|---|
| Primer 1 | 416/37779 | 1.09% | 0.0009% |
| Primer 2 | 123/13064 | 0.94% | 0.0045% |
| Primer 3 | 529/50141 | 1.04% | 0.0027% |
| Average | — | 1.02% ± 0.19% | 0.0025% |
| | | (p = 0.0009) | |

### Method Comparison

Two methods are currently used to detect and quantify rare variants, droplet digital PCR (ddPCR) and Sanger sequencing of TOPO (Topoisomerase-based) cloned nucleic acids. Referring to Table 4, the estimated cost of the method described herein (“mosaic validation method”) is about 90% less expensive than ddPCR and 85× less expensive than the Sanger sequencing/TOPO cloning method. Furthermore, the Sanger sequencing/TOPO cloning method is much less sensitive as its lowest level of reliable detection is an alternate allele fraction of 0.5%. While the purported resolution of ddPCR is an alternate allele fraction of 0.1%, it is not reliable for alternate allele fractions of 0.02% that are within the reliable range of the presently disclosed methods.

Additionally, high-throughput Next Generation Sequencing platforms used in the presently disclosed methods can run massive parallel reactions. Conversely, both Sanger Sequencing/TOPO cloning and ddPCR have relatively limited throughput, thereby increasing cost and time requirements. ddPCR, while having higher throughput than the Sanger sequencing/TOPO cloning method, does not enjoy the throughput of the presently described methods. Additionally, ddPCR primers are labeled with a relatively expensive fluorophore.

TABLE 4
Method Comparison

| | | Mosaic Validation | Sanger + TOPO |
|---|---|---|---|
| | ddPCR | Method | Cloning |
| Estimated Cost to | $256 | $35 | $3,004 |
| Validate allele | | | |
| Cost of Ampli- | $250 (1 set) | $27 (3 sets) | $4 (1 set) |
| fication Primers | | | |
| Cost of | $6/triplicate | $8/3 primers | $3,000/mutation |
| Sequencing/ | | | (1,000 colonies |
| Amplification | | | at $3 per colony) |
| Resolution | 0.1% AAF | 0.02% AAF | 0.5% AAF |
| Throughput | Low-medium | High | Low |

### Detecting and Monitoring Disease

The methods described herein can be used for the detection and/or monitoring of a disease. The detection and characterization of disease-associated variants, including somatic mosaic variants, can provide information relevant for diagnosing a disease, determining the progression or regression of disease, and treating disease. For example, when a cancer cell arises after a somatic event, or when circulating tumor cells are present in a subject, the methods described herein can be used to detect of these cells.

A subject having a disease may undergo periodic testing to determine if the number of a diseased cells is increasing, decreasing, or static. For example, a subject that has cancer may determine the alternative allele frequency of a cancer marker present in samples after the cancer is detected or after treatment has begun. Changes in the alternative allele frequency of the cancer marker would indicate a change in the number of cells carrying the marker (e.g., cancer cells) present in the sample. If the alternative allele frequency is greater than that observed in a previous sample, the subject's cancer is likely progressing or not responding effectively to treatment. If the alternative allele frequency remains static relative to an earlier sample, the disease may be responding treatment sufficiently to stop disease progression, but perhaps not to a level sufficient for disease regression or remission. If the alternative allele frequency decreases relative to an earlier sample, the subject's disease may be regressing, and the absence of such cells (i.e., AAF=0) may signify remission.

### Kits and Compositions for Detecting and Characterizing Low Frequency Genetic Variation

In another embodiment, kits and compositions are provided that advantageously allow for the detection and/or quantification of the presence of low frequency genetic variation in a subject sample (e.g., blood or serum). In one embodiment, the kit includes a composition comprising reagents for performing an amplification reaction, including multiple pairs of forward and reverse primers as described herein. In some embodiments, the reagents include nucleotides, labeled nucleotides, a buffer, a cofactor, and/or a polymerase. In some embodiments, the kit comprises a sterile container that contains the amplification reaction reagents; such containers can be boxes, ampoules, bottles, vials, tubes, bags, pouches, blister-packs, or other suitable container forms known in the art. Such containers can be made of plastic, glass, laminated paper, metal foil, or other materials suitable for holding amplification reagents.

In one embodiment, the kit comprises high-quality (PAGE-purified) RNA or DNA-based primers, premixed at proper concentrations. In some embodiments, the kit comprises reagents for biotin labeling for higher sensitivity assays. In some embodiments, the kit comprises a preselected polymerase (e.g., Phusion U if using RNA primers, or another option) with high fidelity (100× improved error rates compared to a reference polymerase (Taq polymerase). In some embodiments, the kit comprises duplicate primers with differing barcodes for testing case/control samples side-by-side. In some embodiments, the kit comprises preselected primers to avoid other mutation sites, non-overlapping binding sites, and the like. In some embodiments, the kit comprises control DNA (e.g., for negative controls). In some embodiments, the kit comprises ddPCR probes for performing ddPCR and sequencing from the same reaction—(i.e., to obtain copy/expression values and genotype correlation).

In another embodiment, the kit includes a composition comprising reagents for performing a sequencing reaction, including nucleic acid molecules that can specifically bind to an adapter as described above. The reagents, in some embodiments, include nucleotides, labeled nucleotides, a buffer, a cofactor, ion spheres comprising the nucleic acid molecule to be sequenced, and/or enzymes for catalyzing the sequencing reaction. In some embodiments, the kit comprises a sterile container that contains the sequencing reaction reagents; such containers are described above.

In some embodiments, the kit comprises compositions for amplification and sequencing as described above. Kits may also include instructions for performing the reactions.

The practice of the present disclosure teaches, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the compositions and methods disclosed herein. Particularly useful techniques for particular embodiments will be discussed in the sections that follow.

The following examples are put forth to provide those of ordinary skill in the art with a complete disclosure and description of how to perform the amplification, sequencing, and quantifying methods presently disclosed, and are not intended to limit the scope of what the inventors regard as their invention.

### EXAMPLES

### Example 1: Detecting Alleles with an Alternate Allele Fraction (AAF) at or Above 0.1%

To identify low frequency genetic variation in a target nucleic acid sequence with an alternate allele fraction (AAF) of 0.1% or greater, three pairs of primers were designed to yield overlapping amplicons. Each pair of primers comprised a forward and a reverse primer, with each primer having a nucleotide sequence complementary to a portion of the target nucleic acid sequence. Each primer had an adapter at or near its 5′ terminus and upstream from its complementary nucleic acid sequence. The adapter's nucleic acid sequence was complementary to a nucleic acid sequence used in a Next Generation Sequencing (NGS) platform, such as Ion Torrent or Illumina's MiSeq. Additionally, the reverse primer for each pair of primers further comprised an index sequence upstream from the primer's complementary nucleic acid sequence that was unique to the pair.

Three distinct amplification reactions were prepared, each comprising one of the three pairs of primers. The reactions comprised 1.0 μM primers, 1× final concentration of 5× Phusion High-fidelity Buffer (NEB), 200 μM dNTPs, 1.0 units of Phusion High-fidelity Polymerase (NEB), and about 25 to 50 ng of template DNA. The reactions were subjected to an initial denaturation step of 30 seconds at 98° C. followed by 20 cycles of 98° C. (denaturing the template DNA) for 10 seconds, 62° C. (annealing the primers to the template nucleic acid) for 20 seconds, and 72° C. (to extend the DNA product) for 30 seconds. After cycling, the reactions were subjected to an additional 10 minutes at 72° C. as a final extension step.

5 μl of each PCR product were then pooled and purified using a ThermoFisher MagJet purification kit (any kit that removes products <100 base pairs in length can be used). The purified reaction products were resuspended in 20 μl of water, mixed, and incubated for two minutes. The reactions were then placed on a magnet for two minutes, and the eluted DNA was removed. About 1 μl was run on a TapeStation or a Bioanalyzer 2100 to confirm quality.

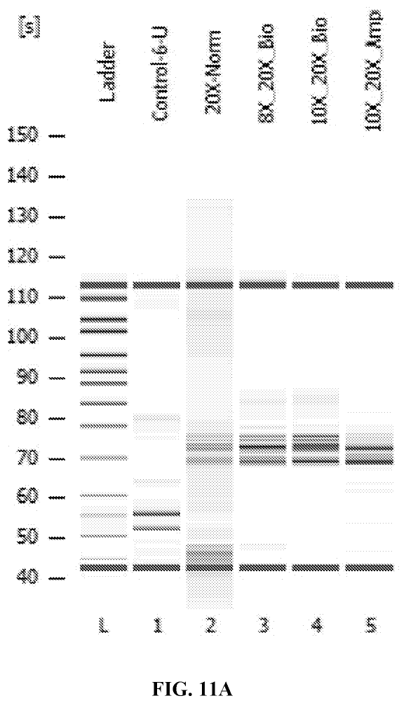

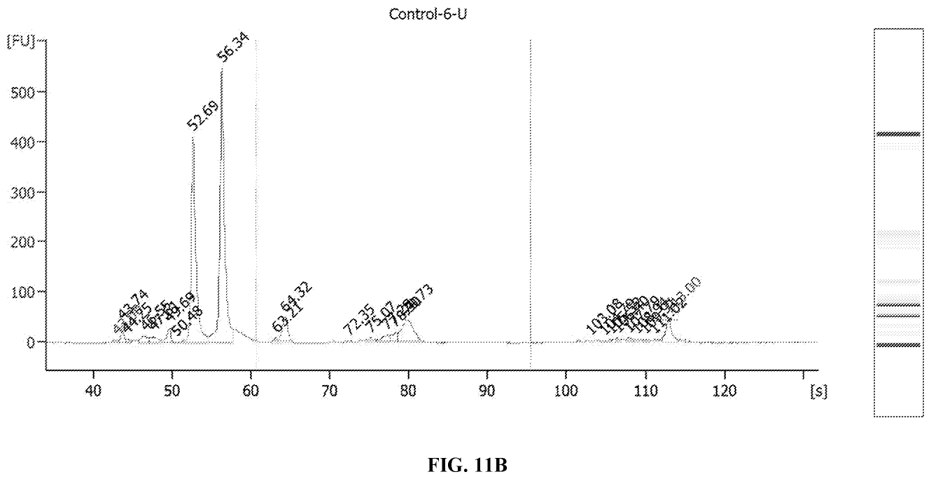

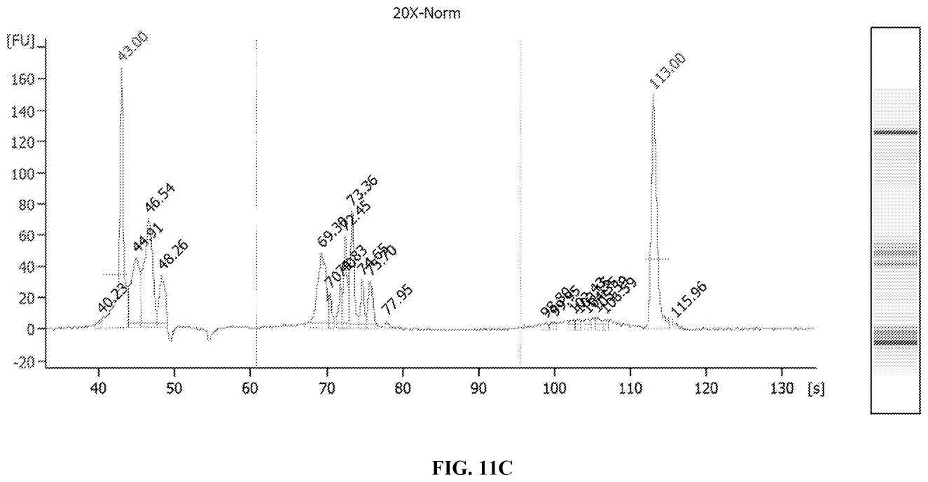

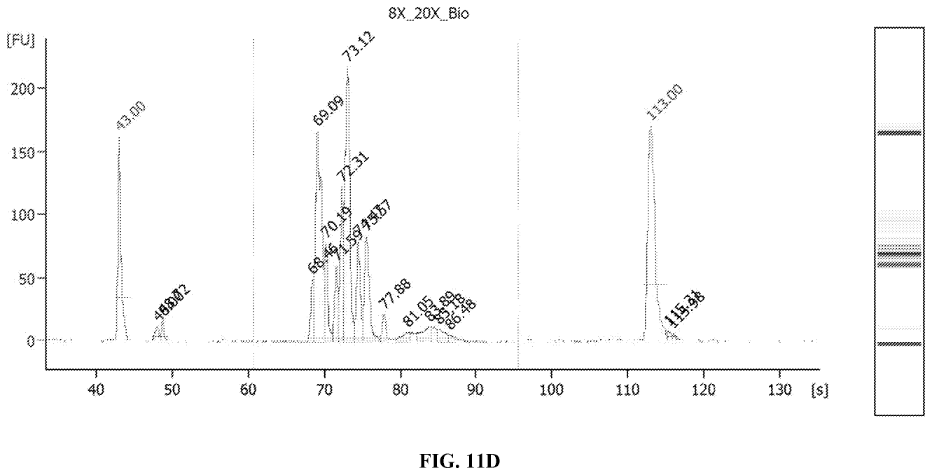

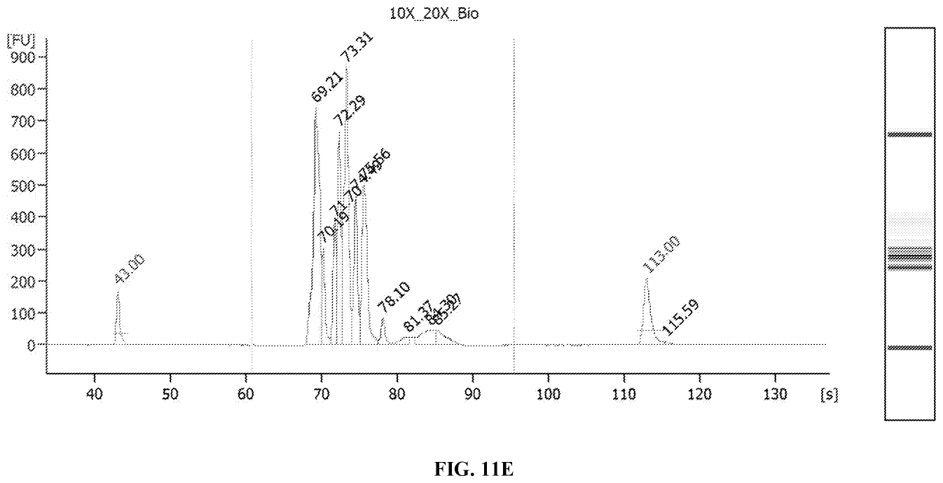

Aliquots of the amplicons generated from a single round of amplification were analyzed on a Bioanalyzer 2100. This amplification strategy yielded detectable amplicons at the expected time point (i.e., between 50 and 60 seconds for the control (FIGS. 11A and 11B) and between 70 and 80 seconds for the amplification performed according to the single round amplification methods described herein (FIGS. 11A and C)). The dark bands at approximately 43 and 113 seconds are control nucleic acids. PicoGreen (ThermoFisher) is then used to measure the concentration of the PCR product, which was subsequently diluted to 100 pM.

The purified PCR reaction products were sequenced using the Ion Torrent system (ThermoFisher Scientific) to generate sequencing reads that comprise the nucleic acid sequence of the target nucleic acid. The sequencing reads were demultiplexed, or segregated, into different bins depending on the detected index sequence. Table 5 provides a summary of the observed alternate allele fractions detected using this method.

TABLE 5
Observed alternate allele fractions

| | | | | | | | | Background | Stdev | Variance | Stdev of | Confidence | |
|---|---|---|---|---|---|---|---|---|---|---|---|---|---|
| | | | | | | IT Read | Alt Allele | AAF | Background | Background | Average | interval of | |
| PrimerID | Chr | AlleleStart | Ref | Alt | Gene | Depth | Depth | (within 50 nts) | AAF | AAF | Background | Background | Average AAF |
| 2FLNA_X_153579448_A_G_PH4201_2 | X | 153579448 | A | G | FLNA | 52876 | 331 | 1.02173E−05 | 2.84343E−05 | 7.9943E−10 | 6.34682E−05 | 0.000157664 | 0.006458143 |
| 4SCAF11_12_46321441_T_G_PH4201_1 | 12 | 46321441 | T | G | SCAF11 | 37184 | 129 | 5.45416E−06 | 1.50886E−05 | 2.2511E−10 | 0.00011421 | 0.000283714 | 0.002795873 |
| 5SCAF11_12_46321441_T_G_PH4201_2 | 12 | 46321441 | T | G | SCAF11 | 30037 | 49 | 3.91357E−05 | 0.00010047 | 9.9614E−09 | 0.00011421 | 0.000283714 | 0.002795873 |
| 6SCAF11_12_46321441_T_G_PH4201_3 | 12 | 46321441 | T | G | SCAF11 | 64191 | 211 | 4.41917E−05 | 0.000171234 | 2.8945E−08 | 0.00011421 | 0.000283714 | 0.002795873 |
| 10SLX4_16_3639306_G_A_PH4201_1 | 16 | 3639306 | G | A | SLX4 | 45836 | 265 | 2.45568E−05 | 5.14068E−05 | 2.6139E−09 | 5.74749E−05 | 0.000142776 | 0.003440733 |
| 11SLX4_16_3639306_G_A_PH4201_2 | 16 | 3639306 | G | A | SLX4 | 52791 | 145 | 4.46418E−05 | 7.51276E−05 | 5.5658E−09 | 5.74749E−05 | 0.000142776 | 0.003440733 |
| 12SLX4_16_3639306_G_A_PH4201_3 | 16 | 3639306 | G | A | SLX4 | 41805 | 75 | 1.69855E−05 | 4.18365E−05 | 1.7304E−09 | 5.74749E−05 | 0.000142776 | 0.003440733 |
| 16LAMA3_18_21453038_C_T_PH4201_1 | 18 | 21453038 | C | T | LAMA3 | 44807 | 167 | 1.75378E−05 | 5.46427E−05 | 2.9554E−09 | 8.21671E−05 | 0.000204114 | 0.0038759 |
| 17LAMA3_18_21453038_C_T_PH4201_2 | 18 | 21453038 | C | T | LAMA3 | 10076 | 37 | 3.19472E−05 | 9.90798E−05 | 9.6703E−09 | 8.21671E−05 | 0.000204114 | 0.0038759 |
| 18LAMA3_18_21453038_C_T_PH4201_3 | 18 | 21453038 | C | T | LAMA3 | 46352 | 196 | 6.83075E−05 | 8.78932E−05 | 7.6286E−09 | 8.21671E−05 | 0.000204114 | 0.0038759 |
| 19FLNA_X_153587777_G_C_PH4201_1 | X | 153587777 | G | C | FLNA | 48596 | 289 | 2.0029E−05 | 4.94376E−05 | 2.4189E−09 | 7.59331E−05 | 0.000188628 | 0.0055221 |
| 20FLNA_X_153587777_G_C_PH4201_2 | X | 153587777 | G | C | FLNA | 51421 | 304 | 4.46736E−05 | 0.000116647 | 1.3412E−08 | 7.59331E−05 | 0.000188628 | 0.0055221 |
| 21FLNA_X_153587777_G_C_PH4201_3 | X | 153587777 | G | C | FLNA | 35689 | 168 | 1.85615E−05 | 3.84922E−05 | 1.4664E−09 | 7.59331E−05 | 0.000188628 | 0.0055221 |
| 28SCAF11_12_46321441_T_G_PH4201_1 | 12 | 46321441 | T | G | SCAF11 | 72595 | 141 | 5.63756E−06 | 1.88736E−05 | 3.5221E−10 | 6.8039E−05 | 0.000169018 | 0.001519564 |
| 29SCAF11_12_46321441_T_G_PH4201_2 | 12 | 46321441 | T | G | SCAF11 | 17298 | 17 | 3.91321E−05 | 0.000105292 | 1.0939E−08 | 6.8039E−05 | 0.000169018 | 0.001519564 |
| 30SCAF11_12_46321441_T_G_PH4201_3 | 12 | 46321441 | T | G | SCAF11 | 60601 | 99 | 1.62602E−05 | 5.12726E−05 | 2.5972E−09 | 6.8039E−05 | 0.000169018 | 0.001519564 |
| 34SLX4_16_3639306_G_A_PH4201_1 | 16 | 3639306 | G | A | SLX4 | 71852 | 100 | 2.80354E−05 | 0.000109029 | 1.1752E−08 | 9.16386E−05 | 0.000227643 | 0.001528917 |
| 35SLX4_16_3639306_G_A_PH4201_2 | 16 | 3639306 | G | A | SLX4 | 20195 | 43 | 2.27701E−05 | 0.000109229 | 1.1755E−08 | 9.16386E−05 | 0.000227643 | 0.001528917 |
| 36SLX4_16_3639306_G_A_PH4201_3 | 16 | 3639306 | G | A | SLX4 | 44100 | 47 | 1.88841E−05 | 4.12854E−05 | 1.6851E−09 | 9.16386E−05 | 0.000227643 | 0.001528917 |
| 40LAMA3_18_21453038_C_T_PH4201_1 | 18 | 21453038 | C | T | LAMA3 | 119239 | 348 | 1.75474E−05 | 5.56879E−05 | 3.0695E−09 | 6.79599E−05 | 0.000168822 | 0.001625798 |
| 41LAMA3_18_21453038_C_T_PH4201_2 | 18 | 21453038 | C | T | LAMA3 | 44385 | 27 | 2.89431E−05 | 7.37946E−05 | 5.3721E−09 | 6.79599E−05 | 0.000168822 | 0.001625798 |
| 42LAMA3_18_21453038_C_T_PH4201_3 | 18 | 21453038 | C | T | LAMA3 | 89592 | 121 | 3.1764E−05 | 7.40446E−05 | 5.4141E−09 | 6.79599E−05 | 0.000168822 | 0.001625798 |
| 43FLNA_X_153587777_G_C_PH4201_1 | X | 153587777 | G | C | FLNA | 53971 | 238 | 2.50405E−05 | 6.37295E−05 | 4.0196E−09 | 7.02955E−05 | 0.000174624 | 0.003419407 |
| 44FLNA_X_153587777_G_C_PH4201_2 | X | 153587777 | G | C | FLNA | 70189 | 280 | 3.09214E−05 | 8.27202E−05 | 6.7489E−09 | 7.02955E−05 | 0.000174624 | 0.003419407 |
| 45FLNA_X_153587777_G_C_PH4201_3 | X | 153587777 | G | C | FLNA | 35499 | 66 | 2.36192E−05 | 6.40637E−05 | 4.0559E−09 | 7.02955E−05 | 0.000174624 | 0.003419407 |
| 46ZNF223_19_44571260_C_A_PH4201_1 | 19 | 44571260 | C | A | ZNF223 | 26856 | 0 | 1.20017E−05 | 2.67638E−05 | 7.0851E−10 | 9.09222E−05 | 0.000225863 | 0.000220546 |
| 47ZNF223_19_44571260_C_A_PH4201_2 | 19 | 44571260 | C | A | ZNF223 | 36859 | 0 | 1.57225E−05 | 7.08468E−05 | 4.9557E−09 | 9.09222E−05 | 0.000225863 | 0.000220546 |
| 48ZNF223_19_44571260_C_A_PH4201_3 | 19 | 44571260 | C | A | ZNF223 | 37785 | 25 | 6.62844E−05 | 0.000139445 | 1.9136E−08 | 9.09222E−05 | 0.000225863 | 0.000220546 |
| 49FLNA_X_153579448_A_G_PH4201_1 | X | 153579448 | A | G | FLNA | 50890 | 95 | 9.53088E−06 | 2.75309E−05 | 7.5006E−10 | 6.09297E−05 | 0.000151358 | 0.002228303 |
| 50FLNA_X_153579448_A_G_PH4201_2 | X | 153579448 | A | G | FLNA | 22262 | 61 | 1.76593E−05 | 3.42833E−05 | 1.1615E−09 | 6.09297E−05 | 0.000151358 | 0.002228303 |
| 58SLX4_16_3639306_G_A_PH4201_1 | 16 | 3639306 | G | A | SLX4 | 56582 | 42 | 2.07782E−05 | 7.67312E−05 | 5.82E−09 | 7.90631E−05 | 0.000196404 | 0.001099338 |
| 59SLX4_16_3639306_G_A_PH4201_2 | 16 | 3639306 | G | A | SLX4 | 20626 | 11 | 2.99415E−05 | 0.000102898 | 1.0412E−08 | 7.90631E−05 | 0.000196404 | 0.001099338 |
| 60SLX4_16_3639306_G_A_PH4201_3 | 16 | 3639306 | G | A | SLX4 | 17306 | 35 | 2.07858E−05 | 5.05071E−05 | 2.5213E−09 | 7.90631E−05 | 0.000196404 | 0.001099338 |
| 67FLNA_X_153587777_G_C_PH4201_1 | X | 153587777 | G | C | FLNA | 104074 | 329 | 1.52718E−05 | 3.30872E−05 | 1.0835E−09 | 6.50076E−05 | 0.000161488 | 0.00207533 |
| 68FLNA_X_153587777_G_C_PH4201_2 | X | 153587777 | G | C | FLNA | 30969 | 60 | 6.13577E−05 | 9.73719E−05 | 9.3284E−09 | 6.50076E−05 | 0.000161488 | 0.00207533 |
| 69FLNA_X_153587777_G_C_PH4201_3 | X | 153587777 | G | C | FLNA | 64753 | 73 | 2.66973E−05 | 4.78617E−05 | 2.2661E−09 | 6.50076E−05 | 0.000161488 | 0.00207533 |
| 70ZNF223_19_44571260_C_A_PH4201_1 | 19 | 44571260 | C | A | ZNF223 | 21369 | 0 | 1.01326E−05 | 2.94338E−05 | 8.5693E−10 | 5.64567E−05 | 0.000140246 | 2.34169E−05 |
| 71ZNF223_19_44571260_C_A_PH4201_2 | 19 | 44571260 | C | A | ZNF223 | 22286 | 1 | 1.64077E−05 | 5.01574E−05 | 2.4839E−09 | 5.64567E−05 | 0.000140246 | 2.34169E−05 |
| 72ZNF223_19_44571260_C_A_PH4201_3 | 19 | 44571260 | C | A | ZNF223 | 39402 | 1 | 2.85368E−05 | 7.94613E−05 | 6.2212E−09 | 5.64567E−05 | 0.000140246 | 2.34169E−05 |
| 82SLX4_16_3639306_G_A_PH4201_1 | 16 | 3639306 | G | A | SLX4 | 38589 | 18 | 2.49275E−05 | 5.35876E−05 | 2.8408E−09 | 6.49472E−05 | 0.000161338 | 0.000400115 |
| 83SLX4_16_3639306_G_A_PH4201_2 | 16 | 3639306 | G | A | SLX4 | 46474 | 20 | 3.26162E−05 | 8.51179E−05 | 7.1416E−09 | 6.49472E−05 | 0.000161338 | 0.000400115 |
| 84SLX4_16_3639306_G_A_PH4201_3 | 16 | 3639306 | G | A | SLX4 | 46122 | 14 | 2.4969E−05 | 5.19921E−05 | 2.6721E−09 | 6.49472E−05 | 0.000161338 | 0.000400115 |
| 91FLNA_X_153587777_G_C_PH4201_1 | X | 153587777 | G | C | FLNA | 57104 | 2 | 1.93602E−05 | 4.88796E−05 | 2.3646E−09 | 5.3862E−05 | 0.000133801 | 0.00083567 |
| 92FLNA_X_153587777_G_C_PH4201_2 | X | 153587777 | G | C | FLNA | 77610 | 5 | 5.43539E−05 | 5.72273E−05 | 3.2301E−09 | 5.3862E−05 | 0.000133801 | 0.00083567 |
| 93FLNA_X_153587777_G_C_PH4201_3 | X | 153587777 | G | C | FLNA | 33644 | 81 | 2.4105E−05 | 5.60543E−05 | 3.1087E−09 | 5.3862E−05 | 0.000133801 | 0.00083567 |
| 94ZNF223_19_44571260_C_A_PH4201_1 | 19 | 44571260 | C | A | ZNF223 | 50984 | 1 | 1.44169E−05 | 2.46323E−05 | 6.0016E−10 | 5.68753E−05 | 0.000141286 | 0.000110502 |
| 95ZNF223_19_44571260_C_A_PH4201_2 | 19 | 44571260 | C | A | ZNF223 | 85847 | 24 | 5.09988E−05 | 8.53336E−05 | 7.1731E−09 | 5.68753E−05 | 0.000141286 | 0.000110502 |
| 96ZNF223_19_44571260_C_A_PH4201_3 | 19 | 44571260 | C | A | ZNF223 | 30935 | 1 | 2.62988E−05 | 4.43728E−05 | 1.9311E−09 | 5.68753E−05 | 0.000141286 | 0.000110502 |
| 97FLNA_X_153579448_A_G_PH4201_1 | X | 153579448 | A | G | FLNA | 62892 | 1 | 1.12555E−05 | 3.17585E−05 | 9.982E−10 | 4.03517E−05 | 0.000100239 | 2.46952E−05 |
| 98FLNA_X_153579448_A_G_PH4201_2 | X | 153579448 | A | G | FLNA | 68746 | 4 | 1.07011E−05 | 2.88479E−05 | 8.2285E−10 | 4.03517E−05 | 0.000100239 | 2.46952E−05 |
| 99FLNA_X_153579448_A_G_PH4201_3 | X | 153579448 | A | G | FLNA | 27140 | 0 | 2.40557E−05 | 5.56478E−05 | 3.0637E−09 | 4.03517E−05 | 0.000100239 | 2.46952E−05 |
| 100SCAF11_12_46321441_T_G_PH4201_1 | 12 | 46321441 | T | G | SCAF11 | 65401 | 11 | 5.89969E−06 | 2.71282E−05 | 7.2767E−10 | 0.000103346 | 0.000256727 | 6.27575E−05 |
| 101SCAF11_12_46321441_T_G_PH4201_2 | 12 | 46321441 | T | G | SCAF11 | 24696 | 0 | 3.15284E−05 | 8.72093E−05 | 7.5067E−09 | 0.000103346 | 0.000256727 | 6.27575E−05 |
| 102SCAF11_12_46321441_T_G_PH4201_3 | 12 | 46321441 | T | G | SCAF11 | 49802 | 1 | 4.07656E−05 | 0.000155257 | 2.3807E−08 | 0.000103346 | 0.000256727 | 6.27575E−05 |
| 106SLX4_16_3639306_G_A_PH4201_1 | 16 | 3639306 | G | A | SLX4 | 60556 | 41 | 2.11937E−05 | 6.02461E−05 | 3.5901E−09 | 5.85276E−05 | 0.000145391 | 0.000616922 |
| 107SLX4_16_3639306_G_A_PH4201_2 | 16 | 3639306 | G | A | SLX4 | 85121 | 37 | 2.53988E−05 | 7.16005E−05 | 5.0617E−09 | 5.85276E−05 | 0.000145391 | 0.000616922 |
| 108SLX4_16_3639306_G_A_PH4201_3 | 16 | 3639306 | G | A | SLX4 | 33828 | 25 | 1.85644E−05 | 4.05368E−05 | 1.6246E−09 | 5.85276E−05 | 0.000145391 | 0.000616922 |
| 112LAMA3_18_21453038_C_T_PH4201_1 | 18 | 21453038 | C | T | LAMA3 | 141247 | 17 | 1.81195E−05 | 4.55462E−05 | 2.0533E−09 | 0.000187047 | 0.000464651 | 0.000155433 |
| 114LAMA3_18_21453038_C_T_PH4201_3 | 18 | 21453038 | C | T | LAMA3 | 106954 | 37 | 2.62147E−05 | 6.57093E−05 | 4.2637E−09 | 0.000187047 | 0.000464651 | 0.000155433 |
| 115FLNA_X_153587777_G_C_PH4201_1 | X | 153587777 | G | C | FLNA | 48712 | 0 | 1.33842E−05 | 4.73444E−05 | 2.2184E−09 | 6.18101E−05 | 0.000153545 | 0.000046184 |
| 116FLNA_X_153587777_G_C_PH4201_2 | X | 153587777 | G | C | FLNA | 14435 | 2 | 2.92084E−05 | 6.26768E−05 | 3.8746E−09 | 6.18101E−05 | 0.000153545 | 0.000046184 |
| 117FLNA_X_153587777_G_C_PH4201_3 | X | 153587777 | G | C | FLNA | 34613 | 0 | 2.62791E−05 | 7.36629E−05 | 5.3685E−09 | 6.18101E−05 | 0.000153545 | 0.000046184 |
| 118ZNF223_19_44571260_C_A_PH4201_1 | 19 | 44571260 | C | A | ZNF223 | 50603 | 1 | 1.31139E−05 | 3.22556E−05 | 1.0297E−09 | 7.24504E−05 | 0.000179977 | 6.58723E−06 |
| 119ZNF223_19_44571260_C_A_PH4201_2 | 19 | 44571260 | C | A | ZNF223 | 42129 | 0 | 2.51869E−05 | 0.000102637 | 1.0399E−08 | 7.24504E−05 | 0.000179977 | 6.58723E−06 |
| 120ZNF223_19_44571260_C_A_PH4201_3 | 19 | 44571260 | C | A | ZNF223 | 76059 | 0 | 3.28129E−05 | 6.6232E−05 | 4.3181E−09 | 7.24504E−05 | 0.000179977 | 6.58723E−06 |
| 124SCAF11_12_46321441_T_G_PH4201_1 | 12 | 46321441 | T | G | SCAF11 | 81594 | 0 | 7.56131E−06 | 2.77648E−05 | 7.6222E−10 | 6.80237E−05 | 0.00016898 | 4.57173E−06 |
| 125SCAF11_12_46321441_T_G_PH4201_2 | 12 | 46321441 | T | G | SCAF11 | 82193 | 0 | 3.60845E−05 | 8.55122E−05 | 7.2174E−09 | 6.80237E−05 | 0.00016898 | 4.57173E−06 |
| 126SCAF11_12_46321441_T_G_PH4201_3 | 12 | 46321441 | T | G | SCAF11 | 72912 | 1 | 3.25164E−05 | 7.73035E−05 | 5.9021E−09 | 6.80237E−05 | 0.00016898 | 4.57173E−06 |
| 130SLX4_16_3639306_G_A_PH4201_1 | 16 | 3639306 | G | A | SLX4 | 27339 | 10 | 3.40321E−05 | 7.19695E−05 | 5.1227E−09 | 6.33819E−05 | 0.000157449 | 0.000283253 |
| 131SLX4_16_3639306_G_A_PH4201_2 | 16 | 3639306 | G | A | SLX4 | 67412 | 31 | 2.06402E−05 | 6.86263E−05 | 4.6484E−09 | 6.33819E−05 | 0.000157449 | 0.000283253 |
| 132SLX4_16_3639306_G_A_PH4201_3 | 16 | 3639306 | G | A | SLX4 | 41457 | 1 | 2.35701E−05 | 4.80335E−05 | 2.2807E−09 | 6.33819E−05 | 0.000157449 | 0.000283253 |
| 136LAMA3_18_21453038_C_T_PH4201_1 | 18 | 21453038 | C | T | LAMA3 | 116508 | 90 | 1.83925E−05 | 5.41278E−05 | 2.8999E−09 | 5.91213E−05 | 0.000146865 | 0.00031725 |
| 137LAMA3_18_21453038_C_T_PH4201_2 | 18 | 21453038 | C | T | LAMA3 | 91222 | 4 | 2.2096E−05 | 6.01332E−05 | 3.5735E−09 | 5.91213E−05 | 0.000146865 | 0.00031725 |
| 138LAMA3_18_21453038_C_T_PH4201_3 | 18 | 21453038 | C | T | LAMA3 | 59074 | 8 | 2.90008E−05 | 6.37446E−05 | 4.0126E−09 | 5.91213E−05 | 0.000146865 | 0.00031725 |
| 139FLNA_X_153587777_G_C_PH4201_1 | X | 153587777 | G | C | FLNA | 48033 | 3 | 2.29118E−05 | 5.18672E−05 | 2.6625E−09 | 5.62488E−05 | 0.00013973 | 3.21058E−05 |
| 140FLNA_X_153587777_G_C_PH4201_2 | X | 153587777 | G | C | FLNA | 61361 | 0 | 3.44416E−05 | 7.64576E−05 | 5.761E−09 | 5.62488E−05 | 0.00013973 | 3.21058E−05 |
| 141FLNA_X_153587777_G_C_PH4201_3 | X | 153587777 | G | C | FLNA | 29533 | 1 | 1.53183E−05 | 3.28539E−05 | 1.0683E−09 | 5.62488E−05 | 0.00013973 | 3.21058E−05 |
| 190NA_5_173266954_G_A_A-pancreas_1 | 5 | 173266954 | G | A | NA | 106354 | 134 | 6.35041E−05 | 0.000307 | 9.3213E−08 | 0.000187713 | 0.000466304 | 0.000853995 |
| 196NA_5_173266954_G_A_A-pons_2 | 5 | 173266954 | G | A | NA | 122112 | 2681 | 3.94563E−05 | 9.45171E−05 | 8.8128E−09 | 0.000117275 | 0.000291328 | 0.020158133 |
| 199NA_5_173266954_G_A_A-pancreas_2 | 5 | 173266954 | G | A | NA | 93898 | 51 | 4.59757E−05 | 0.00010127 | 1.0122E−08 | 0.000187713 | 0.000466304 | 0.000853995 |
| 205NA_5_173266954_G_A_A-pons_3 | 5 | 173266954 | G | A | NA | 39129 | 799 | 6.47892E−05 | 0.000119659 | 1.3993E−08 | 0.000117275 | 0.000291328 | 0.020158133 |
| 208NA_5_173266954_G_A_A-pancreas_3 | 5 | 173266954 | G | A | NA | 51390 | 39 | 3.69912E−05 | 4.92248E−05 | 2.3726E−09 | 0.000187713 | 0.000466304 | 0.000853995 |
| 212NA_11_49854989_C_T_A-17_3 | 11 | 49854989 | C | T | NA | 24985 | 4 | 2.43233E−05 | 6.79798E−05 | 4.5716E−09 | 5.29008E−05 | 0.000131413 | 0.000080048 |
| 213NA_11_49854989_C_T_A-17_1 | 11 | 49854989 | C | T | NA | 95864 | 0 | 1.34233E−05 | 3.22146E−05 | 1.0254E−09 | 5.29008E−05 | 0.000131413 | 0.000080048 |
| 4SCAF11_12_46321441_T_G_PH4201_1 | 12 | 46321441 | T | G | SCAF11 | 42588 | 20869 | 3.69888E−06 | 1.05458E−05 | 1.0996E−10 | 9.31199E−05 | 0.000231323 | 0.491051667 |
| 5SCAF11_12_46321441_T_G_PH4201_2 | 12 | 46321441 | T | G | SCAF11 | 13886 | 6743 | 6.22253E−05 | 0.000150646 | 2.2396E−08 | 9.31199E−05 | 0.000231323 | 0.491051667 |
| 6SCAF11_12_46321441_T_G_PH4201_3 | 12 | 46321441 | T | G | SCAF11 | 54414 | 27073 | 2.36841E−05 | 5.95921E−05 | 3.5084E−09 | 9.31199E−05 | 0.000231323 | 0.491051667 |
| 10SLX4_16_3639306_G_A_PH4201_1 | 16 | 3639306 | G | A | SLX4 | 32196 | 18299 | 2.91067E−05 | 6.47837E−05 | 4.1528E−09 | 6.1556E−05 | 0.000152914 | 0.542452333 |
| 11SLX4_16_3639306_G_A_PH4201_2 | 16 | 3639306 | G | A | SLX4 | 22986 | 12241 | 2.16324E−05 | 6.40091E−05 | 4.0453E−09 | 6.1556E−05 | 0.000152914 | 0.542452333 |
| 12SLX4_16_3639306_G_A_PH4201_3 | 16 | 3639306 | G | A | SLX4 | 47406 | 24957 | 2.72861E−05 | 5.66025E−05 | 3.1694E−09 | 6.1556E−05 | 0.000152914 | 0.542452333 |
| 19FLNA_X_153587777_G_C_PH4201_1 | X | 153587777 | G | C | FLNA | 44686 | 20278 | 2.41014E−05 | 5.75831E−05 | 3.282E−09 | 9.8689E−05 | 0.000245157 | 0.485348667 |
| 20FLNA_X_153587777_G_C_PH4201_2 | X | 153587777 | G | C | FLNA | 64553 | 33761 | 5.76983E−05 | 0.000153505 | 2.3241E−08 | 9.8689E−05 | 0.000245157 | 0.485348667 |
| 21FLNA_X_153587777_G_C_PH4201_3 | X | 153587777 | G | C | FLNA | 38524 | 18463 | 2.10532E−05 | 5.21856E−05 | 2.6956E−09 | 9.8689E−05 | 0.000245157 | 0.485348667 |
| 25FLNA_X_153579448_A_G_PH4201_1 | X | 153579448 | A | G | FLNA | 114923 | 33468 | 9.44768E−06 | 2.42237E−05 | 5.8074E−10 | 2.96956E−05 | 7.3768E−05 | 0.213852 |
| 26FLNA_X_153579448_A_G_PH4201_2 | X | 153579448 | A | G | FLNA | 91936 | 21769 | 1.45941E−05 | 3.33324E−05 | 1.0994E−09 | 2.96956E−05 | 7.3768E−05 | 0.213852 |
| 27FLNA_X_153579448_A_G_PH4201_3 | X | 153579448 | A | G | FLNA | 38714 | 4396 | 1.18446E−05 | 3.1243E−05 | 9.654E−10 | 2.96956E−05 | 7.3768E−05 | 0.213852 |
| 28SCAF11_12_46321441_T_G_PH4201_1 | 12 | 46321441 | T | G | SCAF11 | 89492 | 11917 | 4.87432E−05 | 8.54665E−05 | 7.2225E−09 | 8.6509E−05 | 0.0002149 | 0.132397333 |
| 29SCAF11_12_46321441_T_G_PH4201_2 | 12 | 46321441 | T | G | SCAF11 | 17322 | 2203 | 3.81643E−05 | 0.00011398 | 1.2821E−08 | 8.6509E−05 | 0.0002149 | 0.132397333 |
| 30SCAF11_12_46321441_T_G_PH4201_3 | 12 | 46321441 | T | G | SCAF11 | 98948 | 13541 | 1.49858E−05 | 4.9336E−05 | 2.4084E−09 | 8.6509E−05 | 0.0002149 | 0.132397333 |
| 40LAMA3_18_21453038_C_T_PH4201_1 | 18 | 21453038 | C | T | LAMA3 | 120826 | 18663 | 1.83113E−05 | 6.70941E−05 | 4.4557E−09 | 8.97691E−05 | 0.000222999 | 0.143225 |
| 41LAMA3_18_21453038_C_T_PH4201_2 | 18 | 21453038 | C | T | LAMA3 | 80326 | 10141 | 3.60021E−05 | 0.000115684 | 1.3223E−08 | 8.97691E−05 | 0.000222999 | 0.143225 |
| 42LAMA3_18_21453038_C_T_PH4201_3 | 18 | 21453038 | C | T | LAMA3 | 96768 | 14415 | 3.92469E−05 | 8.11081E−05 | 6.4963E−09 | 8.97691E−05 | 0.000222999 | 0.143225 |
| 43FLNA_X_153587777_G_C_PH4201_1 | X | 153587777 | G | C | FLNA | 95577 | 17749 | 1.93432E−05 | 5.30161E−05 | 2.782E−09 | 6.76114E−05 | 0.000167956 | 0.19288 |
| 44FLNA_X_153587777_G_C_PH4201_2 | X | 153587777 | G | C | FLNA | 103754 | 20188 | 4.62967E−05 | 9.98995E−05 | 9.8432E−09 | 6.76114E−05 | 0.000167956 | 0.19288 |
| 45FLNA_X_153587777_G_C_PH4201_3 | X | 153587777 | G | C | FLNA | 40366 | 8007 | 1.29684E−05 | 3.31758E−05 | 1.0887E−09 | 6.76114E−05 | 0.000167956 | 0.19288 |
| 46ZNF223_19_44571260_C_A_PH4201_1 | 19 | 44571260 | C | A | ZNF223 | 55900 | 4 | 1.48408E−05 | 2.97638E−05 | 8.7625E−10 | 6.19546E−05 | 0.000153904 | 0.000137168 |
| 47ZNF223_19_44571260_C_A_PH4201_2 | 19 | 44571260 | C | A | ZNF223 | 47820 | 5 | 1.77988E−05 | 6.33744E−05 | 3.9655E−09 | 6.19546E−05 | 0.000153904 | 0.000137168 |
| 48ZNF223_19_44571260_C_A_PH4201_3 | 19 | 44571260 | C | A | ZNF223 | 46731 | 11 | 3.29939E−05 | 8.22195E−05 | 6.6734E−09 | 6.19546E−05 | 0.000153904 | 0.000137168 |
| 52SCAF11_12_46321441_T_G_PH4201_1 | 12 | 46321441 | T | G | SCAF11 | 70921 | 4630 | 1.46677E−05 | 3.74441E−05 | 1.385E−09 | 6.34898E−05 | 0.000157717 | 0.0605694 |
| 53SCAF11_12_46321441_T_G_PH4201_2 | 12 | 46321441 | T | G | SCAF11 | 74343 | 4407 | 3.11607E−05 | 9.06887E−05 | 8.1176E−09 | 6.34898E−05 | 0.000157717 | 0.0605694 |
| 54SCAF11_12_46321441_T_G_PH4201_3 | 12 | 46321441 | T | G | SCAF11 | 132680 | 7582 | 1.65308E−05 | 5.11932E−05 | 2.5903E−09 | 6.34898E−05 | 0.000157717 | 0.0605694 |
| 64LAMA3_18_21453038_C_T_PH4201_1 | 18 | 21453038 | C | T | LAMA3 | 136420 | 9410 | 1.60786E−05 | 4.93645E−05 | 2.412E−09 | 7.02577E−05 | 0.00017453 | 0.0648923 |
| 65LAMA3_18_21453038_C_T_PH4201_2 | 18 | 21453038 | C | T | LAMA3 | 95136 | 5138 | 2.3178E−05 | 7.34881E−05 | 5.3354E−09 | 7.02577E−05 | 0.00017453 | 0.0648923 |
| 66LAMA3_18_21453038_C_T_PH4201_3 | 18 | 21453038 | C | T | LAMA3 | 103917 | 7450 | 3.96786E−05 | 8.45598E−05 | 7.061E−09 | 7.02577E−05 | 0.00017453 | 0.0648923 |
| 67FLNA_X_153587777_G_C_PH4201_1 | X | 153587777 | G | C | FLNA | 102340 | 8898 | 2.23604E−05 | 5.06317E−05 | 2.5374E−09 | 7.2709E−05 | 0.000180619 | 0.087697467 |
| 68FLNA_X_153587777_G_C_PH4201_2 | X | 153587777 | G | C | FLNA | 89619 | 8593 | 5.4927E−05 | 0.000109454 | 1.1807E−08 | 7.2709E−05 | 0.000180619 | 0.087697467 |
| 69FLNA_X_153587777_G_C_PH4201_3 | X | 153587777 | G | C | FLNA | 64276 | 5159 | 1.58968E−05 | 3.91355E−05 | 1.5158E−09 | 7.2709E−05 | 0.000180619 | 0.087697467 |
| 70ZNF223_19_44571260_C_A_PH4201_1 | 19 | 44571260 | C | A | ZNF223 | 44181 | 2 | 1.44693E−05 | 2.98906E−05 | 8.8363E−10 | 5.28463E−05 | 0.000131278 | 3.4157E−05 |
| 71ZNF223_19_44571260_C_A_PH4201_2 | 19 | 44571260 | C | A | ZNF223 | 52445 | 3 | 1.4025E−05 | 4.72239E−05 | 2.2019E−09 | 5.28463E−05 | 0.000131278 | 3.4157E−05 |
| 72ZNF223_19_44571260_C_A_PH4201_3 | 19 | 44571260 | C | A | ZNF223 | 45498 | 0 | 2.60957E−05 | 7.32218E−05 | 5.2927E−09 | 5.28463E−05 | 0.000131278 | 3.4157E−05 |
| 73FLNA_X_153579448_A_G_PH4201_1 | X | 153579448 | A | G | FLNA | 107654 | 6311 | 1.06204E−05 | 2.82324E−05 | 7.8877E−10 | 3.45601E−05 | 8.58522E−05 | 0.045955333 |
| 74FLNA_X_153579448_A_G_PH4201_2 | X | 153579448 | A | G | FLNA | 103932 | 4578 | 1.35519E−05 | 3.80512E−05 | 1.4316E−09 | 3.45601E−05 | 8.58522E−05 | 0.045955333 |
| 75FLNA_X_153579448_A_G_PH4201_3 | X | 153579448 | A | G | FLNA | 57423 | 2021 | 1.2331E−05 | 3.71279E−05 | 1.3628E−09 | 3.45601E−05 | 8.58522E−05 | 0.045955333 |
| 82SLX4_16_3639306_G_A_PH4201_1 | 16 | 3639306 | G | A | SLX4 | 114402 | 3794 | 4.05151E−05 | 6.0057E−05 | 3.5689E−09 | 9.85036E−05 | 0.000244696 | 0.031055833 |
| 83SLX4_16_3639306_G_A_PH4201_2 | 16 | 3639306 | G | A | SLX4 | 122229 | 3343 | 2.16981E−05 | 6.56154E−05 | 4.2509E−09 | 9.85036E−05 | 0.000244696 | 0.031055833 |
| 84SLX4_16_3639306_G_A_PH4201_3 | 16 | 3639306 | G | A | SLX4 | 107799 | 3520 | 4.92961E−05 | 0.00014669 | 2.1289E−08 | 9.85036E−05 | 0.000244696 | 0.031055833 |
| 88LAMA3_18_21453038_C_T_PH4201_1 | 18 | 21453038 | C | T | LAMA3 | 141739 | 3518 | 1.6245E−05 | 4.75211E−05 | 2.2352E−09 | 6.60986E−05 | 0.000164198 | 0.031337033 |
| 89LAMA3_18_21453038_C_T_PH4201_2 | 18 | 21453038 | C | T | LAMA3 | 123130 | 4064 | 1.96342E−05 | 5.26053E−05 | 2.7372E−09 | 6.60986E−05 | 0.000164198 | 0.031337033 |
| 90LAMA3_18_21453038_C_T_PH4201_3 | 18 | 21453038 | C | T | LAMA3 | 96504 | 3492 | 4.10938E−05 | 9.07612E−05 | 8.1346E−09 | 6.60986E−05 | 0.000164198 | 0.031337033 |
| 91FLNA_X_153587777_G_C_PH4201_1 | X | 153587777 | G | C | FLNA | 120137 | 5731 | 1.54135E−05 | 3.71281E−05 | 1.3644E−09 | 5.67267E−05 | 0.000140917 | 0.0437276 |
| 92FLNA_X_153587777_G_C_PH4201_2 | X | 153587777 | G | C | FLNA | 144879 | 6360 | 3.3529E−05 | 8.33444E−05 | 6.8511E−09 | 5.67267E−05 | 0.000140917 | 0.0437276 |
| 93FLNA_X_153587777_G_C_PH4201_3 | X | 153587777 | G | C | FLNA | 78221 | 3096 | 2.01674E−05 | 3.81206E−05 | 1.4382E−09 | 5.67267E−05 | 0.000140917 | 0.0437276 |
| 94ZNF223_19_44571260_C_A_PH4201_1 | 19 | 44571260 | C | A | ZNF223 | 42947 | 2 | 1.60091E−05 | 4.18515E−05 | 1.7333E−09 | 5.59136E−05 | 0.000138897 | 3.31067E−05 |
| 95ZNF223_19_44571260_C_A_PH4201_2 | 19 | 44571260 | C | A | ZNF223 | 65985 | 0 | 1.81489E−05 | 5.1892E−05 | 2.6559E−09 | 5.59136E−05 | 0.000138897 | 3.31067E−05 |
| 96ZNF223_19_44571260_C_A_PH4201_3 | 19 | 44571260 | C | A | ZNF223 | 56871 | 3 | 3.58294E−05 | 7.11016E−05 | 4.9898E−09 | 5.59136E−05 | 0.000138897 | 3.31067E−05 |
| 97FLNA_X_153579448_A_G_PH4201_1 | X | 153579448 | A | G | FLNA | 112175 | 4374 | 7.44648E−06 | 2.09631E−05 | 4.3492E−10 | 2.97837E−05 | 7.39868E−05 | 0.030000867 |
| 98FLNA_X_153579448_A_G_PH4201_2 | X | 153579448 | A | G | FLNA | 112537 | 3127 | 1.3643E−05 | 3.99966E−05 | 1.5827E−09 | 2.97837E−05 | 7.39868E−05 | 0.030000867 |
| 99FLNA_X_153579448_A_G_PH4201_3 | X | 153579448 | A | G | FLNA | 74364 | 1727 | 1.24087E−05 | 2.55048E−05 | 6.4357E−10 | 2.97837E−05 | 7.39868E−05 | 0.030000867 |
| 100SCAF11_12_46321441_T_G_PH4201_1 | 12 | 46321441 | T | G | SCAF11 | 105758 | 1337 | 6.32158E−06 | 1.39944E−05 | 1.9364E−10 | 7.30666E−05 | 0.000181507 | 0.012333833 |
| 101SCAF11_12_46321441_T_G_PH4201_2 | 12 | 46321441 | T | G | SCAF11 | 17613 | 203 | 3.59949E−05 | 0.00011086 | 1.213E−08 | 7.30666E−05 | 0.000181507 | 0.012333833 |
| 102SCAF11_12_46321441_T_G_PH4201_3 | 12 | 46321441 | T | G | SCAF11 | 139164 | 1786 | 2.03063E−05 | 6.11195E−05 | 3.6922E−09 | 7.30666E−05 | 0.000181507 | 0.012333833 |
| 112LAMA3_18_21453038_C_T_PH4201_1 | 18 | 21453038 | C | T | LAMA3 | 144251 | 2632 | 1.19654E−05 | 4.35623E−05 | 1.8783E−09 | 0.000123515 | 0.000306828 | 0.011833627 |
| 113LAMA3_18_21453038_C_T_PH4201_2 | 18 | 21453038 | C | T | LAMA3 | 13958 | 18 | 0.000131449 | 0.000195468 | 3.7537E−08 | 0.000123515 | 0.000306828 | 0.011833627 |
| 114LAMA3_18_21453038_C_T_PH4201_3 | 18 | 21453038 | C | T | LAMA3 | 126650 | 2022 | 3.34354E−05 | 8.02018E−05 | 6.3519E−09 | 0.000123515 | 0.000306828 | 0.011833627 |
| 115FLNA_X_153587777_G_C_PH4201_1 | X | 153587777 | G | C | FLNA | 117886 | 2782 | 1.16224E−05 | 2.79385E−05 | 7.726E−10 | 8.47043E−05 | 0.000210417 | 0.020187733 |
| 117FLNA_X_153587777_G_C_PH4201_3 | X | 153587777 | G | C | FLNA | 82453 | 1404 | 1.44532E−05 | 3.62043E−05 | 1.2972E−09 | 8.47043E−05 | 0.000210417 | 0.020187733 |
| 118ZNF223_19_44571260_C_A_PH4201_1 | 19 | 44571260 | C | A | ZNF223 | 46424 | 0 | 1.75212E−05 | 3.98215E−05 | 1.5687E−09 | 6.15054E−05 | 0.000152788 | 2.88507E−05 |
| 119ZNF223_19_44571260_C_A_PH4201_2 | 19 | 44571260 | C | A | ZNF223 | 48037 | 0 | 1.41639E−05 | 5.2454E−05 | 2.7166E−09 | 6.15054E−05 | 0.000152788 | 2.88507E−05 |
| 120ZNF223_19_44571260_C_A_PH4201_3 | 19 | 44571260 | C | A | ZNF223 | 46215 | 4 | 3.01323E−05 | 8.45884E−05 | 7.0635E−09 | 6.15054E−05 | 0.000152788 | 2.88507E−05 |
| 121FLNA_X_153579448_A_G_PH4201_1 | X | 153579448 | A | G | FLNA | 104910 | 1543 | 1.23152E−05 | 3.51728E−05 | 1.2241E−09 | 4.31322E−05 | 0.000107146 | 0.011726387 |
| 122FLNA_X_153579448_A_G_PH4201_2 | X | 153579448 | A | G | FLNA | 87067 | 1273 | 2.03354E−05 | 6.03457E−05 | 3.6011E−09 | 4.31322E−05 | 0.000107146 | 0.011726387 |
| 123FLNA_X_153579448_A_G_PH4201_3 | X | 153579448 | A | G | FLNA | 60679 | 355 | 1.4749E−05 | 2.76533E−05 | 7.5592E−10 | 4.31322E−05 | 0.000107146 | 0.011726387 |
| 124SCAF11_12_46321441_T_G_PH4201_1 | 12 | 46321441 | T | G | SCAF11 | 128949 | 708 | 6.19744E−05 | 0.000144225 | 2.0567E−08 | 9.41463E−05 | 0.000233872 | 0.006252057 |
| 125SCAF11_12_46321441_T_G_PH4201_2 | 12 | 46321441 | T | G | SCAF11 | 86647 | 600 | 2.55061E−05 | 6.46009E−05 | 4.1191E−09 | 9.41463E−05 | 0.000233872 | 0.006252057 |
| 126SCAF11_12_46321441_T_G_PH4201_3 | 12 | 46321441 | T | G | SCAF11 | 138622 | 879 | 1.41111E−05 | 4.38961E−05 | 1.9045E−09 | 9.41463E−05 | 0.000233872 | 0.006252057 |
| 130SLX4_16_3639306_G_A_PH4201_1 | 16 | 3639306 | G | A | SLX4 | 90970 | 706 | 3.60242E−05 | 6.68836E−05 | 4.4258E−09 | 7.93998E−05 | 0.00019724 | 0.00776187 |
| 131SLX4_16_3639306_G_A_PH4201_2 | 16 | 3639306 | G | A | SLX4 | 107933 | 865 | 1.41706E−05 | 5.09717E−05 | 2.5652E−09 | 7.93998E−05 | 0.00019724 | 0.00776187 |
| 132SLX4_16_3639306_G_A_PH4201_3 | 16 | 3639306 | G | A | SLX4 | 124491 | 935 | 4.18812E−05 | 0.000109779 | 1.1922E−08 | 7.93998E−05 | 0.00019724 | 0.00776187 |
| 136LAMA3_18_21453038_C_T_PH4201_1 | 18 | 21453038 | C | T | LAMA3 | 145645 | 1114 | 1.19383E−05 | 4.08667E−05 | 1.653E−09 | 7.02583E−05 | 0.000174531 | 0.007249317 |
| 137LAMA3_18_21453038_C_T_PH4201_2 | 18 | 21453038 | C | T | LAMA3 | 94621 | 562 | 3.00284E−05 | 6.81186E−05 | 4.5849E−09 | 7.02583E−05 | 0.000174531 | 0.007249317 |
| 138LAMA3_18_21453038_C_T_PH4201_3 | 18 | 21453038 | C | T | LAMA3 | 56742 | 463 | 3.56723E−05 | 9.31623E−05 | 8.5707E−09 | 7.02583E−05 | 0.000174531 | 0.007249317 |
| 139FLNA_X_153587777_G_C_PH4201_1 | X | 153587777 | G | C | FLNA | 78250 | 822 | 1.89315E−05 | 3.97105E−05 | 1.5608E−09 | 4.86093E−05 | 0.000120752 | 0.01034064 |
| 140FLNA_X_153587777_G_C_PH4201_2 | X | 153587777 | G | C | FLNA | 135680 | 1295 | 2.90505E−05 | 6.76937E−05 | 4.5197E−09 | 4.86093E−05 | 0.000120752 | 0.01034064 |
| 141FLNA_X_153587777_G_C_PH4201_3 | X | 153587777 | G | C | FLNA | 90316 | 991 | 1.53494E−05 | 3.19139E−05 | 1.0081E−09 | 4.86093E−05 | 0.000120752 | 0.01034064 |
| 142ZNF223_19_44571260_C_A_PH4201_1 | 19 | 44571260 | C | A | ZNF223 | 45173 | 140 | 3.13548E−05 | 4.1112E−05 | 1.6728E−09 | 5.27123E−05 | 0.000130945 | 0.001166528 |
| 143ZNF223_19_44571260_C_A_PH4201_2 | 19 | 44571260 | C | A | ZNF223 | 66182 | 2 | 1.31595E−05 | 4.74401E−05 | 2.2221E−09 | 5.27123E−05 | 0.000130945 | 0.001166528 |
| 144ZNF223_19_44571260_C_A_PH4201_3 | 19 | 44571260 | C | A | ZNF223 | 32418 | 12 | 2.60104E−05 | 6.71356E−05 | 4.4409E−09 | 5.27123E−05 | 0.000130945 | 0.001166528 |
| 182NA_5_73717969_G_A_C-17_3 | 5 | 73717969 | G | A | NA | 133924 | 9487 | 1.61668E−05 | 5.32435E−05 | 2.8056E−09 | 6.87898E−05 | 0.000170883 | 0.0728698 |
| 183NA_5_73717969_G_A_C-18_3 | 5 | 73717969 | G | A | NA | 148542 | 15441 | 1.06255E−05 | 4.24466E−05 | 1.7833E−09 | 5.751E−05 | 0.000142863 | 0.105353 |
| 184NA_5_73717969_G_A_C-9_3 | 5 | 73717969 | G | A | NA | 149125 | 16289 | 1.72387E−05 | 4.24724E−05 | 1.7855E−09 | 9.5885E−05 | 0.000238192 | 0.1056935 |
| 185NA_5_73717969_G_A_C-11_3 | 5 | 73717969 | G | A | NA | 149150 | 16863 | 1.20897E−05 | 4.73091E−05 | 2.2153E−09 | 5.2303E−05 | 0.000129928 | 0.1146715 |
| 187NA_5_73717969_G_A_C-45_3 | 5 | 73717969 | G | A | NA | 148515 | 18657 | 1.03337E−05 | 4.0947E−05 | 1.6596E−09 | 5.20036E−05 | 0.000129184 | 0.122451 |
| 189NA_5_73717969_G_A_C-17_1 | 5 | 73717969 | G | A | NA | 127236 | 8870 | 3.7739E−05 | 0.000106012 | 1.1096E−08 | 6.87898E−05 | 0.000170883 | 0.0728698 |
| 190NA_5_73717969_G_A_C-18_1 | 5 | 73717969 | G | A | NA | 128246 | 13691 | 3.03114E−05 | 6.99529E−05 | 4.8315E−09 | 5.751E−05 | 0.000142863 | 0.105353 |
| 191NA_5_73717969_G_A_C-9_1 | 5 | 73717969 | G | A | NA | 126424 | 12915 | 4.73437E−05 | 0.000129674 | 1.6602E−08 | 9.5885E−05 | 0.000238192 | 0.1056935 |
| 192NA_5_73717969_G_A_C-11_1 | 5 | 73717969 | G | A | NA | 129169 | 15020 | 2.79676E−05 | 5.7425E−05 | 3.2559E−09 | 5.2303E−05 | 0.000129928 | 0.1146715 |
| 194NA_5_73717969_G_A_C-45_1 | 5 | 73717969 | G | A | NA | 127861 | 15251 | 2.9291E−05 | 6.16219E−05 | 3.7492E−09 | 5.20036E−05 | 0.000129184 | 0.122451 |
| 196NA_5_73717969_G_A_C-17_2 | 5 | 73717969 | G | A | NA | 146571 | 11441 | 6.38217E−06 | 1.72436E−05 | 2.9425E−10 | 6.87898E−05 | 0.000170883 | 0.0728698 |
| 199NA_11_49854989_C_T_A-9_3 | 11 | 49854989 | C | T | NA | 32775 | 3 | 1.75167E−05 | 4.59566E−05 | 2.0895E−09 | 3.59978E−05 | 8.94235E−05 | 4.57666E−05 |
| 200NA_11_49854989_C_T_A-9_1 | 11 | 49854989 | C | T | NA | 141978 | 0 | 1.14214E−05 | 2.25275E−05 | 5.0215E−10 | 3.59978E−05 | 8.94235E−05 | 4.57666E−05 |
| 204NA_1_170130646_T_G_C-9_3 | 1 | 170130646 | T | G | NA | 147507 | 0 | 1.36308E−05 | 3.00914E−05 | 8.896E−10 | 2.98262E−05 | 7.40925E−05 | 0 |
| 1FLNA_X_153579448_A_G_PH4201_1 | X | 153579448 | A | G | FLNA | 24264 | 287 | 1.84946E−05 | 3.64352E−05 | 1.3124E−09 | 3.04522E−05 | 7.56476E−05 | 0.007530833 |
| 2FLNA_X_153579448_A_G_PH4201_2 | X | 153579448 | A | G | FLNA | 69733 | 543 | 5.2092E−06 | 1.02847E−05 | 1.0461E−10 | 3.04522E−05 | 7.56476E−05 | 0.007530833 |
| 3FLNA_X_153579448_A_G_PH4201_3 | X | 153579448 | A | G | FLNA | 65828 | 196 | 1.1365E−05 | 3.71572E−05 | 1.365E−09 | 3.04522E−05 | 7.56476E−05 | 0.007530833 |
| 4SCAF11_12_46321441_T_G_PH4201_1 | 12 | 46321441 | T | G | SCAF11 | 81926 | 249 | 3.69937E−06 | 9.58745E−06 | 9.0886E−11 | 8.90779E−05 | 0.000221282 | 0.003211453 |
| 5SCAF11_12_46321441_T_G_PH4201_2 | 12 | 46321441 | T | G | SCAF11 | 15296 | 51 | 3.52445E−05 | 9.67775E−05 | 9.2426E−09 | 8.90779E−05 | 0.000221282 | 0.003211453 |
| 6SCAF11_12_46321441_T_G_PH4201_3 | 12 | 46321441 | T | G | SCAF11 | 133402 | 435 | 2.51523E−05 | 0.000120985 | 1.4471E−08 | 8.90779E−05 | 0.000221282 | 0.003211453 |
| 10SLX4_16_3639306_G_A_PH4201_1 | 16 | 3639306 | G | A | SLX4 | 58364 | 322 | 2.67548E−05 | 7.55218E−05 | 5.6429E−09 | 6.73416E−05 | 0.000167286 | 0.00393089 |
| 11SLX4_16_3639306_G_A_PH4201_2 | 16 | 3639306 | G | A | SLX4 | 44842 | 110 | 2.6859E−05 | 6.34548E−05 | 3.9749E−09 | 6.73416E−05 | 0.000167286 | 0.00393089 |
| 12SLX4_16_3639306_G_A_PH4201_3 | 16 | 3639306 | G | A | SLX4 | 59385 | 227 | 3.10302E−05 | 6.34771E−05 | 3.9869E−09 | 6.73416E−05 | 0.000167286 | 0.00393089 |
| 16LAMA3_18_21453038_C_T_PH4201_1 | 18 | 21453038 | C | T | LAMA3 | 95634 | 485 | 2.04848E−05 | 7.29925E−05 | 5.2735E−09 | 6.74429E−05 | 0.000167537 | 0.004350827 |
| 17LAMA3_18_21453038_C_T_PH4201_2 | 18 | 21453038 | C | T | LAMA3 | 37744 | 145 | 2.20973E−05 | 5.66442E−05 | 3.1614E−09 | 6.74429E−05 | 0.000167537 | 0.004350827 |
| 18LAMA3_18_21453038_C_T_PH4201_3 | 18 | 21453038 | C | T | LAMA3 | 75615 | 313 | 2.89559E−05 | 7.26407E−05 | 5.2107E−09 | 6.74429E−05 | 0.000167537 | 0.004350827 |
| 19FLNA_X_153587777_G_C_PH4201_1 | X | 153587777 | G | C | FLNA | 91142 | 673 | 9.17142E−06 | 2.32548E−05 | 5.3527E−10 | 9.82176E−05 | 0.000243986 | 0.006361423 |
| 20FLNA_X_153587777_G_C_PH4201_2 | X | 153587777 | G | C | FLNA | 99527 | 756 | 5.65447E−05 | 0.000164486 | 2.668E−08 | 9.82176E−05 | 0.000243986 | 0.006361423 |
| 21FLNA_X_153587777_G_C_PH4201_3 | X | 153587777 | G | C | FLNA | 79186 | 325 | 1.4775E−05 | 4.17448E−05 | 1.7248E−09 | 9.82176E−05 | 0.000243986 | 0.006361423 |
| 22ZNF223_19_44571260_C_A_PH4201_1 | 19 | 44571260 | C | A | ZNF223 | 46605 | 1 | 8.77945E−06 | 2.22494E−05 | 4.8988E−10 | 5.00911E−05 | 0.000124433 | 1.26338E−05 |
| 23ZNF223_19_44571260_C_A_PH4201_2 | 19 | 44571260 | C | A | ZNF223 | 70797 | 0 | 1.24727E−05 | 4.14129E−05 | 1.693E−09 | 5.00911E−05 | 0.000124433 | 1.26338E−05 |
| 24ZNF223_19_44571260_C_A_PH4201_3 | 19 | 44571260 | C | A | ZNF223 | 60811 | 1 | 2.99432E−05 | 7.36047E−05 | 5.3444E−09 | 5.00911E−05 | 0.000124433 | 1.26338E−05 |
| 25FLNA_X_153579448_A_G_PH4201_1 | X | 153579448 | A | G | FLNA | 129429 | 464 | 8.16419E−06 | 2.69819E−05 | 7.2052E−10 | 2.30552E−05 | 5.72723E−05 | 0.00236643 |
| 26FLNA_X_153579448_A_G_PH4201_2 | X | 153579448 | A | G | FLNA | 95869 | 199 | 1.05279E−05 | 1.80926E−05 | 3.2386E−10 | 2.30552E−05 | 5.72723E−05 | 0.00236643 |
| 27FLNA_X_153579448_A_G_PH4201_3 | X | 153579448 | A | G | FLNA | 59087 | 85 | 1.40795E−05 | 2.35888E−05 | 5.5025E−10 | 2.30552E−05 | 5.72723E−05 | 0.00236643 |
| 28SCAF11_12_46321441_T_G_PH4201_1 | 12 | 46321441 | T | G | SCAF11 | 83268 | 112 | 4.55364E−06 | 1.50852E−05 | 2.2501E−10 | 4.26917E−05 | 0.000106052 | 0.001588223 |
| 30SCAF11_12_46321441_T_G_PH4201_3 | 12 | 46321441 | T | G | SCAF11 | 117362 | 122 | 1.16431E−05 | 3.55168E−05 | 1.2473E−09 | 4.26917E−05 | 0.000106052 | 0.001588223 |
| 34SLX4_16_3639306_G_A_PH4201_1 | 16 | 3639306 | G | A | SLX4 | 88998 | 170 | 2.1575E−05 | 6.08845E−05 | 3.6657E−09 | 8.8215E−05 | 0.000219138 | 0.001983499 |
| 35SLX4_16_3639306_G_A_PH4201_2 | 16 | 3639306 | G | A | SLX4 | 67120 | 56 | 4.66127E−05 | 0.000122221 | 1.4746E−08 | 8.8215E−05 | 0.000219138 | 0.001983499 |
| 36SLX4_16_3639306_G_A_PH4201_3 | 16 | 3639306 | G | A | SLX4 | 61759 | 198 | 3.13336E−05 | 7.0612E−05 | 4.9336E−09 | 8.8215E−05 | 0.000219138 | 0.001983499 |
| 43FLNA_X_153587777_G_C_PH4201_1 | X | 153587777 | G | C | FLNA | 107980 | 337 | 1.79554E−05 | 3.73283E−05 | 1.3792E−09 | 4.81874E−05 | 0.000119704 | 0.00332788 |
| 44FLNA_X_153587777_G_C_PH4201_2 | X | 153587777 | G | C | FLNA | 132263 | 411 | 2.41059E−05 | 6.49675E−05 | 4.163E−09 | 4.81874E−05 | 0.000119704 | 0.00332788 |
| 45FLNA_X_153587777_G_C_PH4201_3 | X | 153587777 | G | C | FLNA | 68704 | 258 | 1.47833E−05 | 3.79376E−05 | 1.424E−09 | 4.81874E−05 | 0.000119704 | 0.00332788 |
| 46ZNF223_19_44571260_C_A_PH4201_1 | 19 | 44571260 | C | A | ZNF223 | 57574 | 0 | 1.03416E−05 | 2.74209E−05 | 7.4407E−10 | 5.19426E−05 | 0.000129032 | 6.7653E−06 |
| 47ZNF223_19_44571260_C_A_PH4201_2 | 19 | 44571260 | C | A | ZNF223 | 69787 | 0 | 1.12838E−05 | 4.2106E−05 | 1.7502E−09 | 5.19426E−05 | 0.000129032 | 6.7653E−06 |
| 48ZNF223_19_44571260_C_A_PH4201_3 | 19 | 44571260 | C | A | ZNF223 | 49271 | 1 | 3.33871E−05 | 7.53571E−05 | 5.5998E−09 | 5.19426E−05 | 0.000129032 | 6.7653E−06 |
| 49FLNA_X_153579448_A_G_PH4201_1 | X | 153579448 | A | G | FLNA | 87941 | 197 | 1.1037E−05 | 2.80666E−05 | 7.7953E−10 | 2.67942E−05 | 6.65605E−05 | 0.001935883 |
| 50FLNA_X_153579448_A_G_PH4201_2 | X | 153579448 | A | G | FLNA | 85699 | 173 | 1.1882E−05 | 2.72096E−05 | 7.3257E−10 | 2.67942E−05 | 6.65605E−05 | 0.001935883 |
| 51FLNA_X_153579448_A_G_PH4201_3 | X | 153579448 | A | G | FLNA | 52298 | 81 | 1.08666E−05 | 2.54719E−05 | 6.4169E−10 | 2.67942E−05 | 6.65605E−05 | 0.001935883 |
| 52SCAF11_12_46321441_T_G_PH4201_1 | 12 | 46321441 | T | G | SCAF11 | 54590 | 56 | 1.5484E−05 | 3.75274E−05 | 1.3907E−09 | 6.01151E−05 | 0.000149334 | 0.000855501 |
| 53SCAF11_12_46321441_T_G_PH4201_2 | 12 | 46321441 | T | G | SCAF11 | 35287 | 30 | 3.05225E−05 | 8.63868E−05 | 7.3658E−09 | 6.01151E−05 | 0.000149334 | 0.000855501 |
| 54SCAF11_12_46321441_T_G_PH4201_3 | 12 | 46321441 | T | G | SCAF11 | 130340 | 90 | 1.40915E−05 | 4.59206E−05 | 2.085E−09 | 6.01151E−05 | 0.000149334 | 0.000855501 |
| 67FLNA_X_153587777_G_C_PH4201_1 | X | 153587777 | G | C | FLNA | 119586 | 240 | 1.18964E−05 | 3.5427E−05 | 1.2423E−09 | 6.63438E−05 | 0.000164807 | 0.00198682 |
| 68FLNA_X_153587777_G_C_PH4201_2 | X | 153587777 | G | C | FLNA | 95355 | 226 | 4.46509E−05 | 0.000105779 | 1.1032E−08 | 6.63438E−05 | 0.000164807 | 0.00198682 |
| 69FLNA_X_153587777_G_C_PH4201_3 | X | 153587777 | G | C | FLNA | 56838 | 90 | 1.16134E−05 | 3.06666E−05 | 9.3065E−10 | 6.63438E−05 | 0.000164807 | 0.00198682 |
| 70ZNF223_19_44571260_C_A_PH4201_1 | 19 | 44571260 | C | A | ZNF223 | 59122 | 0 | 1.20736E−05 | 2.8092E−05 | 7.8094E−10 | 4.38919E−05 | 0.000109033 | 0 |
| 71ZNF223_19_44571260_C_A_PH4201_2 | 19 | 44571260 | C | A | ZNF223 | 64656 | 0 | 1.0793E−05 | 3.9434E−05 | 1.5354E−09 | 4.38919E−05 | 0.000109033 | 0 |
| 72ZNF223_19_44571260_C_A_PH4201_3 | 19 | 44571260 | C | A | ZNF223 | 58814 | 0 | 2.39246E−05 | 5.92561E−05 | 3.4632E−09 | 4.38919E−05 | 0.000109033 | 0 |
| 73FLNA_X_153579448_A_G_PH4201_1 | X | 153579448 | A | G | FLNA | 86773 | 167 | 9.98466E−06 | 2.56786E−05 | 6.5252E−10 | 3.30698E−05 | 8.21499E−05 | 0.001185302 |
| 74FLNA_X_153579448_A_G_PH4201_2 | X | 153579448 | A | G | FLNA | 72101 | 70 | 1.20489E−05 | 3.55212E−05 | 1.2474E−09 | 3.30698E−05 | 8.21499E−05 | 0.001185302 |
| 75FLNA_X_153579448_A_G_PH4201_3 | X | 153579448 | A | G | FLNA | 42393 | 28 | 1.63782E−05 | 3.73709E−05 | 1.3809E−09 | 3.30698E−05 | 8.21499E−05 | 0.001185302 |
| 88LAMA3_18_21453038_C_T_PH4201_1 | 18 | 21453038 | C | T | LAMA3 | 82365 | 74 | 1.89137E−05 | 5.99713E−05 | 3.5599E−09 | 6.7586E−05 | 0.000167893 | 0.00088785 |
| 89LAMA3_18_21453038_C_T_PH4201_2 | 18 | 21453038 | C | T | LAMA3 | 73739 | 73 | 2.10443E−05 | 5.8422E−05 | 3.3748E−09 | 6.7586E−05 | 0.000167893 | 0.00088785 |
| 90LAMA3_18_21453038_C_T_PH4201_3 | 18 | 21453038 | C | T | LAMA3 | 98048 | 76 | 3.09949E−05 | 8.27928E−05 | 6.769E−09 | 6.7586E−05 | 0.000167893 | 0.00088785 |
| 91FLNA_X_153587777_G_C_PH4201_1 | X | 153587777 | G | C | FLNA | 96939 | 4 | 1.70301E−05 | 4.40317E−05 | 1.919E−09 | 6.08077E−05 | 0.000151055 | 0.000592997 |
| 92FLNA_X_153587777_G_C_PH4201_2 | X | 153587777 | G | C | FLNA | 108834 | 94 | 3.7497E−05 | 9.10425E−05 | 8.1752E−09 | 6.08077E−05 | 0.000151055 | 0.000592997 |
| 93FLNA_X_153587777_G_C_PH4201_3 | X | 153587777 | G | C | FLNA | 69792 | 61 | 1.29442E−05 | 3.17658E−05 | 9.9855E−10 | 6.08077E−05 | 0.000151055 | 0.000592997 |
| 94ZNF223_19_44571260_C_A_PH4201_1 | 19 | 44571260 | C | A | ZNF223 | 59496 | 0 | 1.31575E−05 | 2.61998E−05 | 6.7928E−10 | 5.65725E−05 | 0.000140534 | 0 |
| 95ZNF223_19_44571260_C_A_PH4201_2 | 19 | 44571260 | C | A | ZNF223 | 60176 | 0 | 1.09985E−05 | 4.22833E−05 | 1.7644E−09 | 5.65725E−05 | 0.000140534 | 0 |
| 96ZNF223_19_44571260_C_A_PH4201_3 | 19 | 44571260 | C | A | ZNF223 | 53680 | 0 | 3.38316E−05 | 8.51887E−05 | 7.1577E−09 | 5.65725E−05 | 0.000140534 | 0 |
| 97FLNA_X_153579448_A_G_PH4201_1 | X | 153579448 | A | G | FLNA | 97371 | 77 | 9.51036E−06 | 2.66569E−05 | 7.0327E−10 | 3.44963E−05 | 8.56935E−05 | 0.000477369 |
| 98FLNA_X_153579448_A_G_PH4201_2 | X | 153579448 | A | G | FLNA | 63812 | 24 | 1.02225E−05 | 2.64331E−05 | 6.9127E−10 | 3.44963E−05 | 8.56935E−05 | 0.000477369 |
| 99FLNA_X_153579448_A_G_PH4201_3 | X | 153579448 | A | G | FLNA | 26394 | 7 | 2.43236E−05 | 4.69184E−05 | 2.1754E−09 | 3.44963E−05 | 8.56935E−05 | 0.000477369 |
| 102SCAF11_12_46321441_T_G_PH4201_3 | 12 | 46321441 | T | G | SCAF11 | 28326 | 0 | 1.8436E−05 | 5.25987E−05 | 2.7355E−09 | 6.68974E−05 | 0.000166182 | 0.000250532 |
| 106SLX4_16_3639306_G_A_PH4201_1 | 16 | 3639306 | G | A | SLX4 | 69218 | 6 | 1.20893E−05 | 3.92501E−05 | 1.5236E−09 | 4.88915E−05 | 0.000121453 | 0.000102273 |
| 107SLX4_16_3639306_G_A_PH4201_2 | 16 | 3639306 | G | A | SLX4 | 38925 | 0 | 1.73624E−05 | 5.98804E−05 | 3.5403E−09 | 4.88915E−05 | 0.000121453 | 0.000102273 |
| 108SLX4_16_3639306_G_A_PH4201_3 | 16 | 3639306 | G | A | SLX4 | 54512 | 12 | 1.79625E−05 | 4.61508E−05 | 2.1072E−09 | 4.88915E−05 | 0.000121453 | 0.000102273 |
| 115FLNA_X_153587777_G_C_PH4201_1 | X | 153587777 | G | C | FLNA | 80552 | 1 | 6.90677E−06 | 2.10375E−05 | 4.3806E−10 | 6.01185E−05 | 0.000149343 | 0.000400965 |
| 117FLNA_X_153587777_G_C_PH4201_3 | X | 153587777 | G | C | FLNA | 58499 | 0 | 9.26507E−06 | 1.90282E−05 | 3.583E−10 | 6.01185E−05 | 0.000149343 | 0.000400965 |
| 119ZNF223_19_44571260_C_A_PH4201_2 | 19 | 44571260 | C | A | ZNF223 | 41307 | 0 | 1.1469E−05 | 4.05857E−05 | 1.6261E−09 | 7.439E−05 | 0.000184795 | 0 |
| 121FLNA_X_153579448_A_G_PH4201_1 | X | 153579448 | A | G | FLNA | 83614 | 23 | 7.52473E−06 | 1.95783E−05 | 3.7928E−10 | 1.73495E−05 | 4.30987E−05 | 0.000191769 |
| 122FLNA_X_153579448_A_G_PH4201_2 | X | 153579448 | A | G | FLNA | 58905 | 9 | 6.48471E−06 | 1.69247E−05 | 2.8319E−10 | 1.73495E−05 | 4.30987E−05 | 0.000191769 |
| 123FLNA_X_153579448_A_G_PH4201_3 | X | 153579448 | A | G | FLNA | 54258 | 8 | 5.38074E−06 | 1.55977E−05 | 2.4055E−10 | 1.73495E−05 | 4.30987E−05 | 0.000191769 |
| 124SCAF11_12_46321441_T_G_PH4201_1 | 12 | 46321441 | T | G | SCAF11 | 10183 | 0 | 1.14584E−05 | 3.89589E−05 | 1.5007E−09 | 5.42166E−05 | 0.000134681 | 4.21327E−05 |
| 125SCAF11_12_46321441_T_G_PH4201_2 | 12 | 46321441 | T | G | SCAF11 | 15823 | 2 | 2.08904E−05 | 7.62989E−05 | 5.7459E−09 | 5.42166E−05 | 0.000134681 | 4.21327E−05 |
| 126SCAF11_12_46321441_T_G_PH4201_3 | 12 | 46321441 | T | G | SCAF11 | 43369 | 0 | 1.05994E−05 | 3.98686E−05 | 1.5716E−09 | 5.42166E−05 | 0.000134681 | 4.21327E−05 |
| 130SLX4_16_3639306_G_A_PH4201_1 | 16 | 3639306 | G | A | SLX4 | 42958 | 8 | 1.40447E−05 | 3.79384E−05 | 1.4238E−09 | 3.76729E−05 | 9.35848E−05 | 0.000135465 |
| 131SLX4_16_3639306_G_A_PH4201_2 | 16 | 3639306 | G | A | SLX4 | 22424 | 2 | 1.55039E−05 | 3.99029E−05 | 1.5721E−09 | 3.76729E−05 | 9.35848E−05 | 0.000135465 |
| 132SLX4_16_3639306_G_A_PH4201_3 | 16 | 3639306 | G | A | SLX4 | 53444 | 7 | 1.75157E−05 | 3.57126E−05 | 1.2618E−09 | 3.76729E−05 | 9.35848E−05 | 0.000135465 |
| 136LAMA3_18_21453038_C_T_PH4201_1 | 18 | 21453038 | C | T | LAMA3 | 121601 | 80 | 9.09633E−06 | 2.94624E−05 | 8.5917E−10 | 5.14128E−05 | 0.000127716 | 0.000420001 |
| 137LAMA3_18_21453038_C_T_PH4201_2 | 18 | 21453038 | C | T | LAMA3 | 53584 | 12 | 2.4705E−05 | 6.33785E−05 | 3.9684E−09 | 5.14128E−05 | 0.000127716 | 0.000420001 |
| 138LAMA3_18_21453038_C_T_PH4201_3 | 18 | 21453038 | C | T | LAMA3 | 47598 | 18 | 2.24107E−05 | 5.60533E−05 | 3.1022E−09 | 5.14128E−05 | 0.000127716 | 0.000420001 |
| 139FLNA_X_153587777_G_C_PH4201_1 | X | 153587777 | G | C | FLNA | 71859 | 2 | 9.87313E−06 | 2.39318E−05 | 5.6689E−10 | 5.99534E−05 | 0.000148933 | 0.000210718 |
| 140FLNA_X_153587777_G_C_PH4201_2 | X | 153587777 | G | C | FLNA | 54664 | 31 | 3.24141E−05 | 9.86693E−05 | 9.6023E−09 | 5.99534E−05 | 0.000148933 | 0.000210718 |
| 141FLNA_X_153587777_G_C_PH4201_3 | X | 153587777 | G | C | FLNA | 53732 | 2 | 9.80512E−06 | 2.49083E−05 | 6.1409E−10 | 5.99534E−05 | 0.000148933 | 0.000210718 |
| 142ZNF223_19_44571260_C_A_PH4201_1 | 19 | 44571260 | C | A | ZNF223 | 31245 | 1 | 1.11919E−05 | 2.51831E−05 | 6.2765E−10 | 0.000189244 | 0.000470108 | 0.000419128 |
| 143ZNF223_19_44571260_C_A_PH4201_2 | 19 | 44571260 | C | A | ZNF223 | 37757 | 0 | 1.22571E−05 | 5.98359E−05 | 3.5344E−09 | 0.000189244 | 0.000470108 | 0.000419128 |
| 144ZNF223_19_44571260_C_A_PH4201_3 | 19 | 44571260 | C | A | ZNF223 | 11425 | 14 | 0.000119374 | 0.000323909 | 1.0328E−07 | 0.000189244 | 0.000470108 | 0.000419128 |
| 182NA_5_73717969_G_A_C-putamen_3 | 5 | 73717969 | G | A | NA | 146868 | 16657 | 1.04913E−05 | 3.63854E−05 | 1.3103E−09 | 7.32249E−05 | 0.000181901 | 0.1121115 |
| 183NA_5_73717969_G_A_C-37_3 | 5 | 73717969 | G | A | NA | 148025 | 16387 | 9.50264E−06 | 3.4586E−05 | 1.184E−09 | 4.9362E−05 | 0.000122622 | 0.1104095 |
| 184NA_5_73717969_G_A_C-7_3 | 5 | 73717969 | G | A | NA | 148518 | 14328 | 1.04058E−05 | 3.78078E−05 | 1.4148E−09 | 6.39402E−05 | 0.000158836 | 0.0963384 |
| 185NA_5_73717969_G_A_C-19_3 | 5 | 73717969 | G | A | NA | 148641 | 19027 | 1.21165E−05 | 4.49393E−05 | 1.9989E−09 | 5.8551E−05 | 0.000145449 | 0.1281165 |
| 186NA_5_73717969_G_A_C-pons_3 | 5 | 73717969 | G | A | NA | 146034 | 15926 | 9.58084E−06 | 3.91421E−05 | 1.5165E−09 | 0.000106587 | 0.000264777 | 0.1114735 |
| 187NA_5_73717969_G_A_C-adrenal_3 | 5 | 73717969 | G | A | NA | 148408 | 18181 | 9.66387E−06 | 4.03626E−05 | 1.6125E−09 | 4.5798E−05 | 0.000113768 | 0.1167425 |
| 188NA_5_73717969_G_A_C-pancreas_3 | 5 | 73717969 | G | A | NA | 139170 | 14360 | 1.77384E−05 | 5.30139E−05 | 2.7812E−09 | 0.000144591 | 0.000359185 | 0.09964535 |
| 189NA_5_73717969_G_A_C-putamen_1 | 5 | 73717969 | G | A | NA | 131453 | 14566 | 4.26031E−05 | 9.76354E−05 | 9.4135E−09 | 7.32249E−05 | 0.000181901 | 0.1121115 |
| 190NA_5_73717969_G_A_C-37_1 | 5 | 73717969 | G | A | NA | 135104 | 14877 | 2.94044E−05 | 6.11223E−05 | 3.6892E−09 | 4.9362E−05 | 0.000122622 | 0.1104095 |
| 191NA_5_73717969_G_A_C-7_1 | 5 | 73717969 | G | A | NA | 132282 | 12726 | 3.32702E−05 | 8.27493E−05 | 6.7619E−09 | 6.39402E−05 | 0.000158836 | 0.0963384 |
| 192NA_5_73717969_G_A_C-19_1 | 5 | 73717969 | G | A | NA | 134589 | 17258 | 2.72636E−05 | 7.01355E−05 | 4.8575E−09 | 5.8551E−05 | 0.000145449 | 0.1281165 |
| 193NA_5_73717969_G_A_C-pons_1 | 5 | 73717969 | G | A | NA | 130810 | 14898 | 4.3965E−05 | 0.000146539 | 2.1205E−08 | 0.000106587 | 0.000264777 | 0.1114735 |
| 194NA_5_73717969_G_A_C-adrenal_1 | 5 | 73717969 | G | A | NA | 134856 | 14966 | 2.2429E−05 | 5.11379E−05 | 2.5824E−09 | 4.5798E−05 | 0.000113768 | 0.1167425 |
| 195NA_5_73717969_G_A_C-pancreas_1 | 5 | 73717969 | G | A | NA | 130978 | 12588 | 5.25575E−05 | 0.000198812 | 3.9032E−08 | 0.000144591 | 0.000359185 | 0.09964535 |
| 196NA_5_73717969_G_A_C-cerebellum_2 | 5 | 73717969 | G | A | NA | 145082 | 21160 | 8.00971E−06 | 2.02271E−05 | 4.0488E−10 | 2.01215E−05 | 4.99846E−05 | 0.145849 |
| 200NA_3_177844577_G_A_C-17_1 | 3 | 177844577 | G | A | NA | 84017 | 46 | 1.38304E−05 | 4.52855E−05 | 2.0272E−09 | 3.85259E−05 | 9.57036E−05 | 0.000361819 |
| 201NA_3_177844577_G_A_C-18_1 | 3 | 177844577 | G | A | NA | 146570 | 343 | 6.03893E−06 | 1.68273E−05 | 2.8015E−10 | 1.33039E−05 | 3.30488E−05 | 0.00252737 |
| 202NA_3_177844577_G_A_C-9_1 | 3 | 177844577 | G | A | NA | 140940 | 1026 | 6.91288E−06 | 2.7459E−05 | 7.4597E−10 | 2.89933E−05 | 7.20232E−05 | 0.007887165 |
| 203NA_3_177844577_G_A_C-11_1 | 3 | 177844577 | G | A | NA | 133617 | 1015 | 1.17129E−05 | 3.84869E−05 | 1.4612E−09 | 2.82699E−05 | 7.02263E−05 | 0.01191867 |
| 204NA_3_177844577_G_A_C-47_1 | 3 | 177844577 | G | A | NA | 146353 | 1231 | 8.90321E−06 | 2.56134E−05 | 6.4921E−10 | 5.15293E−05 | 0.000128006 | 0.010443985 |
| 205NA_3_177844577_G_A_C-45_1 | 3 | 177844577 | G | A | NA | 136805 | 185 | 6.38948E−06 | 3.04637E−05 | 9.1761E−10 | 2.1955E−05 | 5.45392E−05 | 0.001199725 |
| 206NA_3_177844577_G_A_C-44_1 | 3 | 177844577 | G | A | NA | 141268 | 395 | 1.73897E−06 | 5.76108E−06 | 3.2841E−11 | 1.20403E−05 | 2.99097E−05 | 0.002975415 |
| 207NA_3_177844577_G_A_C-17_3 | 3 | 177844577 | G | A | NA | 45421 | 8 | 7.77866E−06 | 3.08413E−05 | 9.4128E−10 | 3.85259E−05 | 9.57036E−05 | 0.000361819 |
| 208NA_3_177844577_G_A_C-18_3 | 3 | 177844577 | G | A | NA | 135197 | 367 | 2.28091E−06 | 8.63841E−06 | 7.3845E−11 | 1.33039E−05 | 3.30488E−05 | 0.00252737 |
| 209NA_3_177844577_G_A_C-9_3 | 3 | 177844577 | G | A | NA | 60391 | 513 | 7.70605E−06 | 3.07423E−05 | 9.3524E−10 | 2.89933E−05 | 7.20232E−05 | 0.007887165 |
| 210NA_3_177844577_G_A_C-11_3 | 3 | 177844577 | G | A | NA | 102580 | 1666 | 3.05559E−06 | 1.17725E−05 | 1.3715E−10 | 2.82699E−05 | 7.02263E−05 | 0.01191867 |
| 211NA_3_177844577_G_A_C-47_3 | 3 | 177844577 | G | A | NA | 26449 | 330 | 3.77525E−05 | 6.86744E−05 | 4.6613E−09 | 5.15293E−05 | 0.000128006 | 0.010443985 |
| 212NA_3_177844577_G_A_C-45_3 | 3 | 177844577 | G | A | NA | 121280 | 127 | 2.40403E−06 | 6.84998E−06 | 4.6433E−11 | 2.1955E−05 | 5.45392E−05 | 0.001199725 |
| 213NA_3_177844577_G_A_C-44_3 | 3 | 177844577 | G | A | NA | 100801 | 318 | 3.94254E−06 | 1.61183E−05 | 2.5709E−10 | 1.20403E−05 | 2.99097E−05 | 0.002975415 |
| 215NA_3_177844577_G_A_C-8_3 | 3 | 177844577 | G | A | NA | 17068 | 94 | 4.22828E−06 | 1.8048E−05 | 3.2234E−10 | 4.47728E−05 | 0.000111222 | 0.0051468 |
| 184SNK383_20_12810118_G_A_SNK383_1 | 20 | 12810118 | G | A | SNK383 | 145741 | 5200 | 1.6912E−05 | 4.04662E−05 | 1.6208E−09 | 4.02592E−05 | 0.000100009 | 0.0356797 |
| 185SNK384_20_12810118_G_A_SNK384_2 | 20 | 12810118 | G | A | SNK384 | 147601 | 5355 | 1.55507E−05 | 3.81814E−05 | 1.4429E−09 | 3.79861E−05 | 9.43628E−05 | 0.0362802 |
| 186SNK385_20_12810118_G_A_SNK385_3 | 20 | 12810118 | G | A | SNK385 | 144097 | 5336 | 2.44978E−05 | 8.29819E−05 | 6.815E−09 | 8.25531E−05 | 0.000205073 | 0.0370306 |
| 188SK215_5_73717969_G_A_SK215_1 | 5 | 73717969 | G | A | SK215 | 145975 | 16363 | 2.01463E−05 | 4.53848E−05 | 2.0383E−09 | 4.51478E−05 | 0.000112153 | 0.112095 |
| 205SNK312_5_173266954_G_A_SNK312_2 | 5 | 173266954 | G | A | SNK312 | 72517 | 1547 | 3.26982E−05 | 7.13994E−05 | 5.028E−09 | 7.09087E−05 | 0.000176147 | 0.0213329 |
| 17NA_5_174228431_G_C_S3PFC_1 | 5 | 174228431 | G | C | NA | 49919 | 1534 | 2.48992E−05 | 4.02286E−05 | 1.6017E−09 | 4.2453E−05 | 0.000105459 | 0.015787505 |
| 18NA_5_174228431_G_C_S3PFC_3 | 5 | 174228431 | G | C | NA | 34311 | 29 | 2.84456E−05 | 4.49986E−05 | 2.0029E−09 | 4.2453E−05 | 0.000105459 | 0.015787505 |
| 19NA_7_283913_T_A_S3PFC_2 | 7 | 283913 | T | A | NA | 18171 | 0 | 5.29407E−05 | 0.000123851 | 1.5172E−08 | 0.000172125 | 0.000427581 | 0 |
| 24NA_9_136638046_C_T_S3PFC_1 | 9 | 136638046 | C | T | NA | 47371 | 4 | 3.05441E−05 | 6.73903E−05 | 4.4756E−09 | 8.8965E−05 | 0.000221001 | 6.6676E−05 |
| 25NA_9_136638046_C_T_S3PFC_2 | 9 | 136638046 | C | T | NA | 41069 | 2 | 3.5752E−05 | 6.04299E−05 | 3.5989E−09 | 8.8965E−05 | 0.000221001 | 6.6676E−05 |
| 26NA_9_136638046_C_T_S3PFC_3 | 9 | 136638046 | C | T | NA | 29900 | 2 | 5.48824E−05 | 0.000126096 | 1.567E−08 | 8.8965E−05 | 0.000221001 | 6.6676E−05 |
| 74NA_2_17125698_C_T_S3PFC_1 | 2 | 17125698 | C | T | NA | 28148 | 3 | 6.7018E−05 | 0.000117751 | 1.3709E−08 | 9.74024E−05 | 0.000241961 | 4.59349E−05 |
| 75NA_2_17125698_C_T_S3PFC_2 | 2 | 17125698 | C | T | NA | 30646 | 0 | 4.8479E−05 | 9.22613E−05 | 8.4165E−09 | 9.74024E−05 | 0.000241961 | 4.59349E−05 |
| 76NA_2_17125698_C_T_S3PFC_3 | 2 | 17125698 | C | T | NA | 32026 | 1 | 5.1095E−05 | 8.00484E−05 | 6.3357E−09 | 9.74024E−05 | 0.000241961 | 4.59349E−05 |
| 103NA_6_79286753_T_C_S3PFC_2 | 6 | 79286753 | T | C | NA | 40164 | 0 | 4.95544E−05 | 9.39536E−05 | 8.727E−09 | 7.51117E−05 | 0.000186588 | 0 |
| 104NA_6_79286753_T_C_S3PFC_3 | 6 | 79286753 | T | C | NA | 44679 | 0 | 3.4306E−05 | 5.0825E−05 | 2.5565E−09 | 7.51117E−05 | 0.000186588 | 0 |
| 111NA_8_40724674_G_A_S3PFC_1 | 8 | 40724674 | G | A | NA | 24021 | 104 | 4.25707E−05 | 9.05005E−05 | 8.105E−09 | 0.000167721 | 0.000416643 | 0.004739067 |
| 112NA_8_40724674_G_A_S3PFC_2 | 8 | 40724674 | G | A | NA | 21527 | 131 | 4.44042E−05 | 7.90849E−05 | 6.1899E−09 | 0.000167721 | 0.000416643 | 0.004739067 |
| 114NA_9_103459386_G_A_S3PFC_1 | 9 | 103459386 | G | A | NA | 32109 | 83 | 5.84782E−05 | 7.6984E−05 | 5.8654E−09 | 6.59462E−05 | 0.00016382 | 0.00265107 |
| 115NA_9_103459386_G_A_S3PFC_2 | 9 | 103459386 | G | A | NA | 30386 | 83 | 5.09081E−05 | 6.53611E−05 | 4.2285E−09 | 6.59462E−05 | 0.00016382 | 0.00265107 |
| 116NA_9_103459386_G_A_S3PFC_3 | 9 | 103459386 | G | A | NA | 86091 | 227 | 6.13687E−05 | 5.46191E−05 | 2.9528E−09 | 6.59462E−05 | 0.00016382 | 0.00265107 |
| 121NA_6_153444080_T_C_S3PFC_3 | 6 | 153444080 | T | C | NA | 58976 | 9758 | 0.000030193 | 8.73841E−05 | 7.5108E−09 | 7.83227E−05 | 0.000194564 | 0.44448 |
| 17NA_5_174228431_G_C_S3PFC_1 | 5 | 174228431 | G | C | NA | 148622 | 149 | 1.24541E−05 | 2.27254E−05 | 5.1083E−10 | 2.26958E−05 | 5.63796E−05 | 0.000685806 |
| 18NA_5_174228431_G_C_S3PFC_3 | 5 | 174228431 | G | C | NA | 75866 | 28 | 1.07793E−05 | 2.2912E−05 | 5.1937E−10 | 2.26958E−05 | 5.63796E−05 | 0.000685806 |
| 19NA_7_283913_T_A_S3PFC_2 | 7 | 283913 | T | A | NA | 25754 | 0 | 1.86353E−05 | 4.34137E−05 | 1.864E−09 | 6.82206E−05 | 0.000169469 | 1.76498E−05 |
| 20NA_7_283913_T_A_S3PFC_3 | 7 | 283913 | T | A | NA | 28329 | 1 | 3.50616E−05 | 8.67623E−05 | 7.4441E−09 | 6.82206E−05 | 0.000169469 | 1.76498E−05 |
| 24NA_9_136638046_C_T_S3PFC_1 | 9 | 136638046 | C | T | NA | 86137 | 0 | 2.67017E−05 | 5.92953E−05 | 3.465E−09 | 7.96953E−05 | 0.000197974 | 4.74267E−05 |
| 25NA_9_136638046_C_T_S3PFC_2 | 9 | 136638046 | C | T | NA | 135595 | 17 | 2.73487E−05 | 7.62347E−05 | 5.7275E−09 | 7.96953E−05 | 0.000197974 | 4.74267E−05 |
| 26NA_9_136638046_C_T_S3PFC_3 | 9 | 136638046 | C | T | NA | 59147 | 1 | 3.56968E−05 | 0.000100033 | 9.8615E−09 | 7.96953E−05 | 0.000197974 | 4.74267E−05 |
| 88NA_22_37475065_G_A_S3PFC_1 | 22 | 37475065 | G | A | NA | 24231 | 1 | 6.34762E−05 | 0.000128382 | 1.6171E−08 | 0.000121471 | 0.000301752 | 1.37565E−05 |
| 89NA_22_37475065_G_A_S3PFC_2 | 22 | 37475065 | G | A | NA | 62505 | 0 | 2.41034E−05 | 5.70967E−05 | 3.225E−09 | 0.000121471 | 0.000301752 | 1.37565E−05 |
| 90NA_22_37475065_G_A_S3PFC_3 | 22 | 37475065 | G | A | NA | 34638 | 0 | 7.69682E−05 | 0.000159271 | 2.487E−08 | 0.000121471 | 0.000301752 | 1.37565E−05 |
| 103NA_6_79286753_T_C_S3PFC_2 | 6 | 79286753 | T | C | NA | 121355 | 0 | 4.01043E−05 | 7.43887E−05 | 5.4708E−09 | 5.96301E−05 | 0.000148129 | 0 |
| 104NA_6_79286753_T_C_S3PFC_3 | 6 | 79286753 | T | C | NA | 86795 | 0 | 2.11582E−05 | 4.0716E−05 | 1.6407E−09 | 5.96301E−05 | 0.000148129 | 0 |
| 111NA_8_40724674_G_A_S3PFC_1 | 8 | 40724674 | G | A | NA | 77763 | 153 | 2.25222E−05 | 5.78709E−05 | 3.3142E−09 | 6.513E−05 | 0.000161792 | 0.0019613 |
| 112NA_8_40724674_G_A_S3PFC_2 | 8 | 40724674 | G | A | NA | 66376 | 108 | 2.61608E−05 | 7.10426E−05 | 4.995E−09 | 6.513E−05 | 0.000161792 | 0.0019613 |
| 113NA_8_40724674_G_A_S3PFC_3 | 8 | 40724674 | G | A | NA | 111825 | 256 | 3.32318E−05 | 6.68025E−05 | 4.4166E−09 | 6.513E−05 | 0.000161792 | 0.0019613 |
| 114NA_9_103459386_G_A_S3PFC_1 | 9 | 103459386 | G | A | NA | 43258 | 251 | 1.78852E−05 | 3.02772E−05 | 9.0726E−10 | 3.42549E−05 | 8.50939E−05 | 0.00484131 |
| 115NA_9_103459386_G_A_S3PFC_2 | 9 | 103459386 | G | A | NA | 68024 | 264 | 1.84993E−05 | 3.55257E−05 | 1.2489E−09 | 3.42549E−05 | 8.50939E−05 | 0.00484131 |
| 116NA_9_103459386_G_A_S3PFC_3 | 9 | 103459386 | G | A | NA | 76644 | 371 | 2.09892E−05 | 3.71224E−05 | 1.364E−09 | 3.42549E−05 | 8.50939E−05 | 0.00484131 |
| 120NA_6_153444080_T_C_S3PFC_1 | 6 | 153444080 | T | C | NA | 38726 | 22027 | 1.20121E−05 | 2.65412E−05 | 6.9089E−10 | 7.80641E−05 | 0.000193922 | 0.30980405 |
| 121NA_6_153444080_T_C_S3PFC_3 | 6 | 153444080 | T | C | NA | 105378 | 5355 | 2.47757E−05 | 0.000108161 | 1.1497E−08 | 7.80641E−05 | 0.000193922 | 0.30980405 |

### Example 2: Detecting Alleles with an Alternate Allele Fraction (AAF) at or Above 0.025%

To identify low frequency genetic variation in a target nucleic acid sequence with an alternate allele fraction of 0.025% or greater, three pairs of primers were designed to yield overlapping amplicons. Each pair of primers comprised a forward and a reverse primer, with each primer having a nucleotide sequence complementary to a portion of the target nucleic acid sequence. Each primer had an adapter at or near its 5′ terminus and upstream from its complementary nucleic acid sequence. The adapter's nucleic acid sequence was complementary to a nucleic acid sequence used in an NGS platform, such as Ion Torrent or Illumina's MiSeq. Each individual reverse primer further comprised an index sequence upstream from the primer's complementary nucleic acid sequence. Additionally, each individual forward or reverse primer in each pair of primers further comprised a unique molecular identifier (UMI). No two primers had the same UMI.

Three distinct amplification reactions were prepared, each comprising one of the three pairs of primers. The reactions comprised 1.0 μM primers, 1× final concentration of 5× Phusion High-fidelity Buffer (NEB), 200 μM deoxynucleotide triphosphates (dNTPs), 0.1 μl of 0.4 mM Biotin-14-dCTP, 1.0 units of Phusion High-fidelity Polymerase (NEB), and about 25 to 50 ng of template DNA. The reactions were subjected to an initial denaturation step of 30 seconds at 98° C. followed by 8 cycles of 98° C. (denaturing the template DNA) 10 seconds, 62° C. (annealing the primers to the template nucleic acid) for 20 seconds, and 72° C. (to extend the DNA product) for 30 seconds. After cycling, the reactions were subjected to an additional 10 minutes at 72° C. as a final extension step. The reaction products, or amplicons, were purified by washing 5 μl of MyOne C1 streptavidin beads two times with 1× Binding-Washing (B&W) buffer and then resuspending the beads in 25 μl of 2×B&W buffer. 25 μl of the MyOne C1 streptavidin beads was then added to 25 μl of the PCR amplicon and incubated at room temperature for 15 minutes with mixing. The mixture was exposed to a magnet, which isolates the beads with the amplicons bound thereto. The supernatant was removed, and 500 μl 1× B&W buffer was added to the beads, mixed, and exposed to the magnet. Again, the supernatant is removed, and the wash was repeated. The beads were finally resuspended in 28 μl water. Some reaction products were purified using an exonuclease 1/shrimp alkaline phosphatase (ExoSap) enzymatic purification protocol, wherein 8 μl of the commercially available ExoSap-It reagent (ThermoFisher) was added to the 20 μl amplification reaction and incubated at 37° C. for 15 minutes followed by 80° C. for 15 minutes.

While the amplicons were attached to the streptavidin beads, an additional amplification was performed to enhance the copy number of the bound amplicons. Briefly, the additional amplification reactions comprised 1.0 μM primers, 1× final concentration of 5× Phusion High-fidelity Buffer (NEB), 200 μM deoxynucleotide triphosphates (dNTPs), 0.1 μl of 0.4 mM Biotin-14-dCTP, 1.0 units of Phusion High-fidelity Polymerase (NEB), and about 25 to 50 ng of template DNA. The reactions were subjected to an initial denaturation step of 30 seconds at 98° C. followed by 20 cycles of 98° C. (denaturing the template DNA) 10 seconds, 62° C. (annealing the primers to the template nucleic acid) for 20 seconds, and 72° C. (to extend the DNA product) for 30 seconds. After cycling, the reactions were subjected to an additional 10 minutes at 72° C. as a final extension step, and 5 μl of the PCR reactions were pooled. A ThermoFisher MagJet purification kit that removes products <100 base pairs in length was used to purify the amplicons. Specifically, the amplicons in the pooled reactions were bound to streptavidin beads, and the supernatant was removed. The beads were then resuspended in 200 of water, mixed, and incubated for two minutes. The mixture was then exposed to a magnet for two minutes, and the eluted DNA was captured.

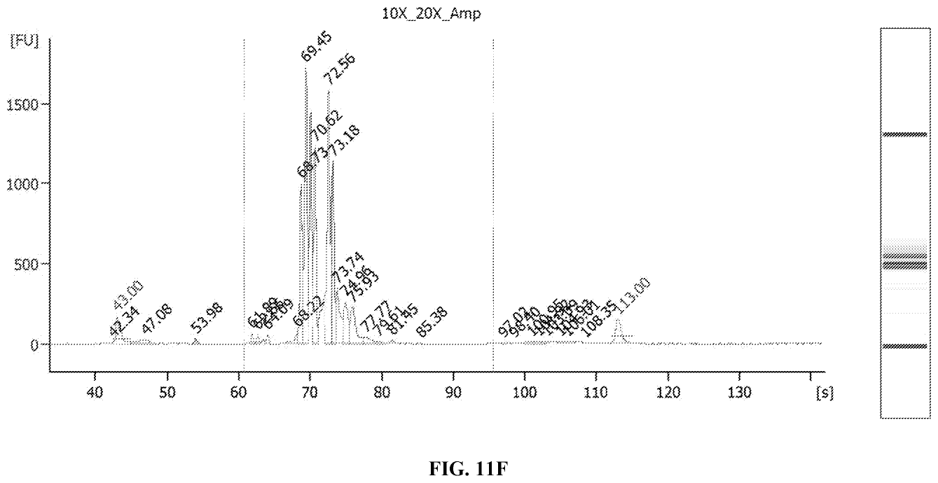

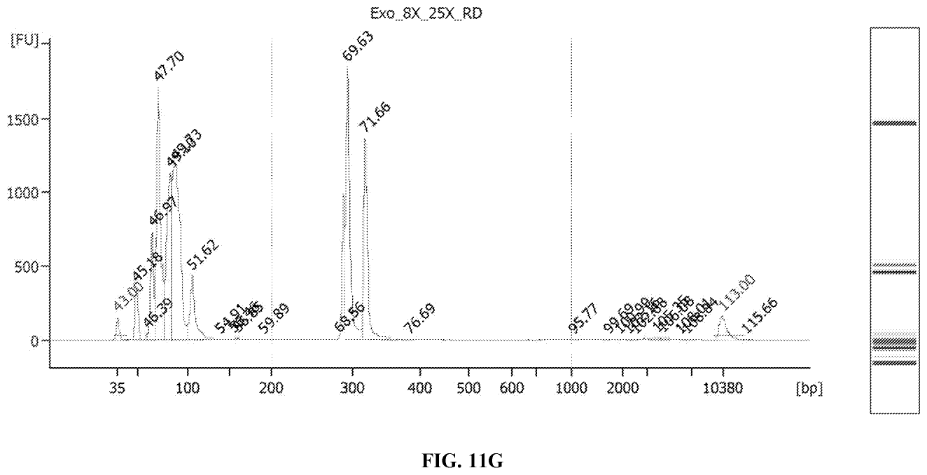

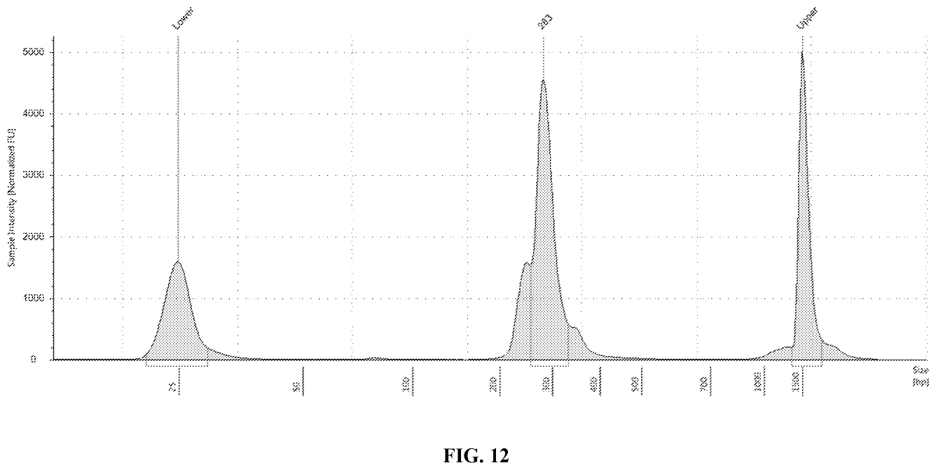

Referring to FIGS. 11D to 11G, 1 μl aliquots of eluted amplicons prepared using two rounds of amplification were run on a Bioanalyzer 2100 to confirm the quality of amplicons for use in downstream sequencing. FIG. 11D (first round=8 cycles; second round=20 cycles; biotin purification), FIG. 11E (first round=10 cycles; second round=20 cycles; biotin purification), FIG. 11F (first round=10 cycles; second round=20 cycles; no biotin purification), and FIG. 11G (first round=8 cycles; second round=25 cycles; ExoSAP purification) all show detectable amounts of the desired amplicons. For comparison purposes, data from an amplicon analyzed using TapeStation is shown in FIG. 12. Less sensitive than the Bioanalyzer 2100, the amplicons detected using the TapeStation are represented by much broader and rounded peaks compared to the Bioanalyzer 2100. However, this approach is still viable for the methods presented herein.

After determining the concentration of the eluted DNA, it was diluted to 100 pM, and the purified PCR reaction products were sequenced using the Ion Torrent system (ThermoFisher Scientific).

### Example 3: Sensitivity and Reproducibility Assessment

The sensitivity and reproducibility of the methods described herein were assessed through serial dilutions of known germline mutations and known somatic mutations across a spectrum of alternative allele fractions. A comparison of alternative allele fractions with other known detections strategies including whole genome sequencing, whole exome sequencing, targeted sequencing, Sanger sequencing with Topo-cloning, and ddPCR was performed. First, triplicate primers (i.e., 3 unique pairs of primers) were designed as described in the methods for known germline mutations occurring in both the autosomal and X-chromosomal regions, including both heterozygous and hemizygous alleles. Twelve serial dilutions were sequenced on the Ion Torrent S5 with 400 base pair reads using six unique barcodes per primer. All reads were processed using custom analytical scripts (described in methods), allowing the for comparison of assessed and expected allelic fractions.

Referring to FIGS. 13 and 14, the methods described herein accurately measured alternative allele fractions as low as 0.025% and up to germline events when using a 50 ng of genomic DNA, although for significant detection above the amplicon-specific error rates, alternative allele fractions were typically required to be above 0.05%. The strong correlation between the expected and assessed alternative allele fractions (R²=0.9995 and R²=0.9761 for dilutions between 0-60% and for dilutions between 0-0.864%, respectively) across the assessed germline alleles, indicates that this method is extremely accurate for low-level alternative allele fractions.

Given that input DNA is often limited but is also known as an important factor for sensitivity for somatic alleles, decreased inputs of DNA were tested to determine if they could achieve a similar level of precision under the same dilution curve. Indeed, while decreased input DNA does impact the sensitivity, alternative allele fractions down to 0.05% remain detectable, though at a slightly elevated standard deviation among the triplicate primes for the lowest alternative allele fractions of 0.05%, indicating that when validating alleles below 0.1% alternative allele fractions, increased input DNA could improve precision. Furthermore, the impact of total sequencing depth on the accuracy was assessed to identify the minimum depth needed for accurate determination of alternative allele fractions. Using random sampling of the initial raw unmapped data, a strong correlation of read depths above threshold level can be made, and sequencing beyond this threshold will provide minimal benefits on the precision of the alternative allele fraction assessment.

### Example 4: Somatic Mosaics in Human Brain Samples

Frozen postmortem human brain specimens from 61 autism spectrum disorder cases and 15 neurotypical controls were obtained for analysis. DNA was extracted from dorsolateral prefrontal cortex where available (or generic cortex in a minority of cases) using lysis buffer from the QIAamp DNA Mini kit (Qiagen) followed by phenol chloroform extraction and isopropanol cleanup. Samples UMB4334, UMB4899, UMB4999, UMB5027, UMB5115, UMB5176, UMB5297, UMB5302, UMB1638, UMB4671, and UMB797 were processed using TruSeq Nano DNA library preparation (Illumina) followed by Illumina HiSeq X Ten sequencing to a minimum 200× depth. All remaining samples were processed using TruSeq DNA PCR-Free library preparation (Illumina) followed by minimum 30× sequencing of seven separate libraries on the Illumina HiSeq X Ten, for a total minimum coverage of 210× per sample. An average of 251× depth was achieved across all samples, using 150 base pair paired-end reads. Two samples, UMB5771 and UMB5939, had parental saliva-derived DNA available, and DNA from both parents for these two cases was obtained and sequenced to about 50× depth. Parental DNA was not available for any other samples. Additionally, DNA was extracted from Brodmann Area 17 (occipital lobe) for cases UMB4638 and UMB4643 and sequenced at Macrogen to a minimum 210× depth following PCR-free library preparation. Bulk heart and liver sequencing data, as well as single-cell sequencing data from three individuals (UMB1465, UMB4643, and UMB4638) were used in this study.

### Mutation Calling and Filtration

All paired-end FASTQ files were aligned using BWA-MEM version 0.7.8 to the GRCh37 human reference genome including the hs37d5 decoy sequence from the Broad Institute, following GATK best practices (software.broadinstitute.org/gatk/best-practices/). Mutect2-PoN was used to generate two pairs of panel-of-normals (PoN) by using 60 autism spectrum disorder samples or 15 control samples to remove sequencing artifacts and germline variants from the other group. Rare variants were further selected by filtering out any variant with a maximum population minor allele frequency >0.001 in any of Kaviar, 1000 Genomes, EVS6500 (evs.gs.washington.edu/EVS/), ExACnonpsych, or gnomAD (gnomad.broadinstitute.org/). Repetitive region variants were removed using RepeatMasker (www.repeatmasker.org/), and variants within segmental duplication regions or shared between multiple individuals were also removed. Low-quality calls tagged “t_lod_fstar,” “str_contraction,” and “triallelic_site” were removed. For analysis of damaging heterozygous variants, variants were identified in the 78 risk genes previously used.

For somatic mutation detection, a minimum alternate (or variant) allele fraction (AAF or VAF) of 0.03 was required unless a variant was phasable by Mutect2, which allowed for rescue of variants down to an alternate allele fraction of 0.02. Low-quality calls tagged “triallelic_site” were removed. A minimum alternate read depth of four reads was required. Only private events among the population were analyzed. An upper alternate allele fraction threshold of 0.40 was set and heterozygous germline variants were removed. Variants within repetitive regions were also removed, leaving 14,984 candidate somatic mutations. MosaicForecast was then used to perform read-backed phasing and identify high-confidence mosaics from the candidate call set. Briefly, features likely to be correlated with mosaic detection specificity were selected: mapping quality, base quality, clustering of mutations, read depth, number of mismatches per read, read1/read2 bias, strand bias, base position, read position, trinucleotide context, sequencing cycle, library preparation method, and genotype likelihood. Based on these features a random forest model was trained using phased variants. Further training was conducted using parental whole genome sequencing data from two cases UMB5771 and UMB5939 as well as single cell whole genome sequencing data from three control brains, UMB1465, UMB4643, and UMB4638 for which inherited germline mutations or variants present in multiple single cells at a low alternate allele fraction (averaging alternate allele fraction <0.30, likely representing sequencing or alignment artifact), supplied a training set of false positives. Predicted mosaics were further filtered by removing genomic regions enriched for low-alternate allele fraction variants and by removing variants with unusually high sequencing depth that also occurred in regions marked as copy number variants (CNVs) by Meerkat. Following all training and filtration, 1143 putative mosaic variants were identified. One autism spectrum disorder sample, MSSM007, was eliminated from the study due to very high noise suggestive of contamination or sequencing artifact.

Pathogenicity prediction scores were calculated for functional mosaic and germline variants using SIFT, PolyPhen-2, MutationTaster, and CADD. To be considered damaging, a variant had to be predicted as damaging or probably damaging (or CADD phred score >20) by at least three out of four prediction tools. Mutations in genes were checked for overlap with the Simons Foundation Autism Research Initiative (SFARI) database of autism spectrum disorder—relevant genes (gene.sfari.org/), and with the Online Inheritance in Man (OMIM) database of genes with relevance to any human disease (www.omim.org/).

### Triple Primer PCR Sequencing

Targeted validation was attempted on 243 of 1143 possible mosaic variants. PCR primers were designed for each variant and synthesized with Ion Torrent adapters P and A, with barcodes added for unique identification. PCR amplification was performed using Phusion HotStart II DNA Polymerase (Thermo) as described by the manufacturer, with 20-25 cycles of amplification. Reactions were pooled and purified with AMPure XP technology (Agencourt), then sequenced on the Ion Torrent Personal Genome Machine using the Ion 530 chip with 400 base pair reads, reaching an average coverage of 118,000 reads per variant amongst reactions that yielded mappable reads. Following demultiplexing and trimming, reads were mapped using BWAMEM (a Burrows-Wheeler aligner algorithm) and locally realigned using GATK. BAM files were then imported into a CLC Genomics workbench (Qiagen) and mosaic variants were identified using the following filters: minimum frequency 0.05%, minimum depth 10,000× per reaction, minimum count 50, required significance 0.1%, central and neighborhood base quality of >15, and 3-nucleotide homopolymer filtration. Variants were then classified as validated true mosaics (198 variants), homozygous reference with variant not present (21 variants), germline heterozygous (1 variant), PCR reactions failed to amplify (19 variants), or undetermined (4 variants). The “undetermined” designation was used for variants for which the originally sequenced DNA was not available, so validation was conducted on a separate DNA extraction that could have slightly different clonal architecture. It was also used to classify two variants in which sequencing noise precluded validation interpretation. Validation success rates were calculated as the number of true mosaics divided by the sum of true mosaics, homozygous reference, and germline heterozygous. Weighted averaging across PCR and PCR-free variant validation was used to determine a comprehensive validation rate of 93%. Five variants from UMB5771 and UMB5939 were also re-sequenced in parent DNA, which confirmed a mosaic state in the offspring and homozygous reference in parents.

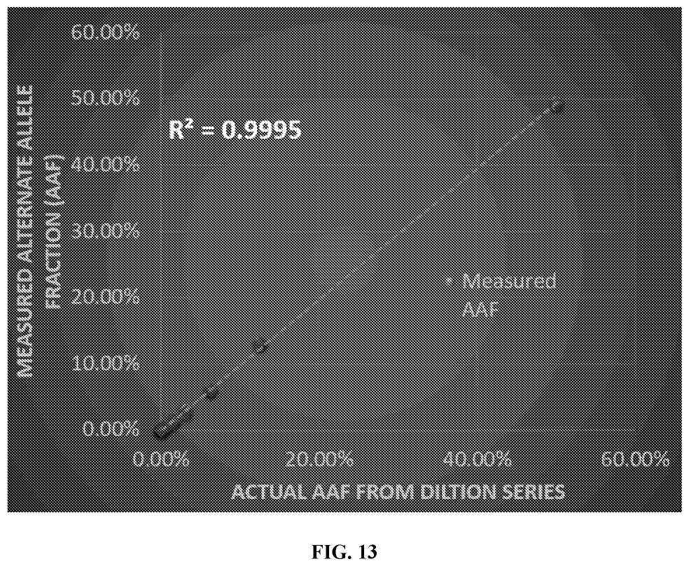

A deleterious missense C to A change in the autism spectrum disorder risk gene CACNA1A was called in 5.2% of sequencing reads in case UMB1174 (FIG. 15). Targeted validation of this region using the methods described herein generated 93,000 reads that confirmed an alternate allele fraction of 5.0%, meaning that this mutation is present in about 10% of cells.

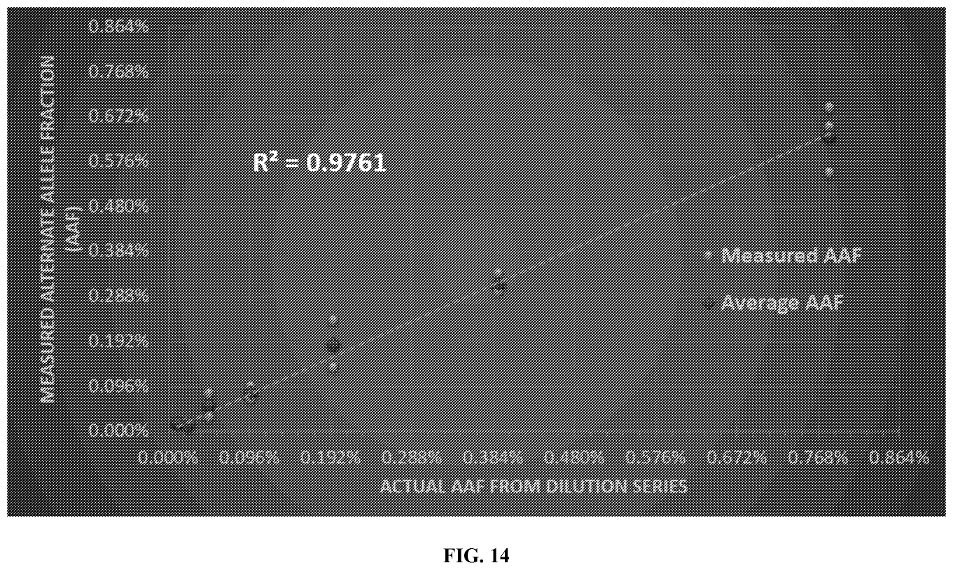

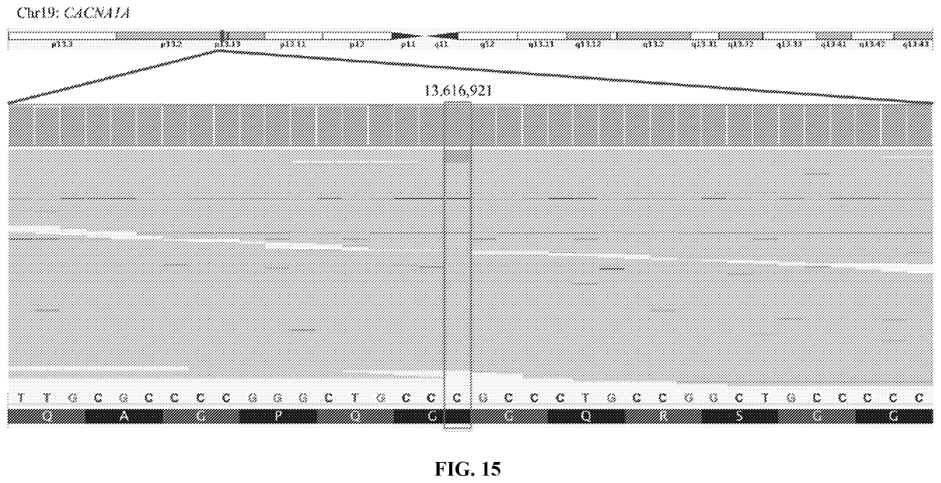

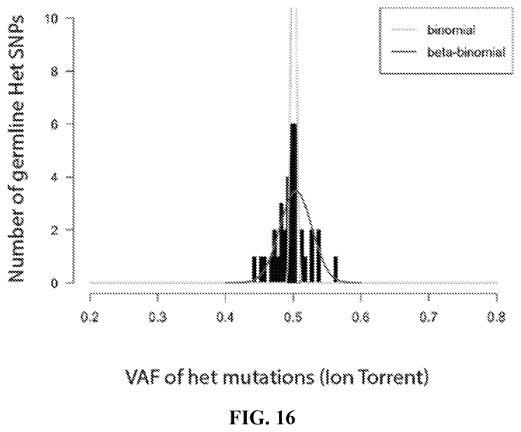

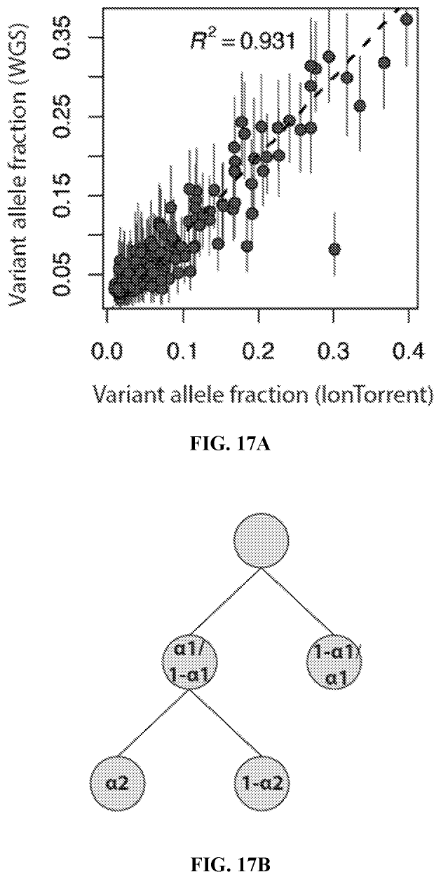

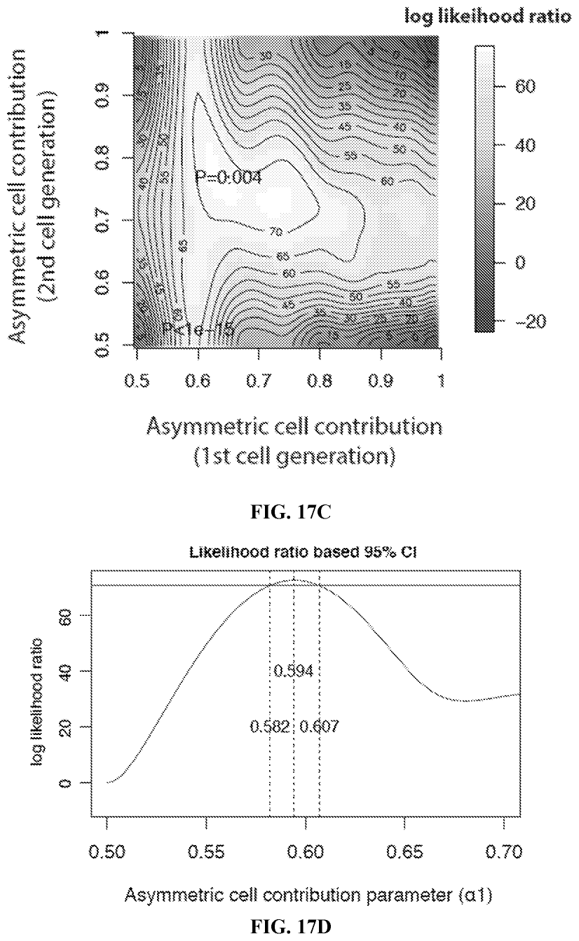

Ion Torrent amplicon resequencing for 34 germline heterozygous mutations revealed that alternate allele frequencies were slightly over-dispersed compared to a binomial distribution (FIG. 16), likely due to noise induced by PCR amplification. The alternate allele frequency distribution was fit with a beta-binomial model to capture the over-dispersion (θ=452.44, p=1/(1+θ)=0.0022). 220 Ion Torrent-validated mosaics was used with a similar model to measure potential asymmetrical cell contributions to the brain during early embryonic development (FIG. 17A). Briefly, α₁ and 1−α₁ were defined as the fraction of brain cells deriving from each of the two cells created by the first division of the brain ancestor cell. A contribution parameter value of α=0.5 meant that the first two cells contributed equally to the brain, while a non-0.5 value meant that the cell contribution was asymmetrical. Given a specific α₁, it was possible to calculate the expected alternate allele frequency for mutations acquired at different branches of the early phylogeny (FIG. 17B). Assuming the mutation rate per cell generation was constant (i.e., the two cell divisions from the 2nd cell generation had the same mutation rate), the likelihood of a mosaic arising on a specific branch was computed by multiplying the estimated sensitivity for detecting mosaics at the expected branch alternate allele frequency with the over-dispersion beta-binomial likelihood of the mosaic alternate allele fraction measured by the deep Ion Torrent sequencing. The log likelihoods for all sites were then summed over all branches to estimate the log likelihood of a specific al. al was fit by maximizing the log likelihood over α₁∈[0.5, 1] using a grid search with step size=0.001. A likelihood ratio test was used to compare the asymmetrical model to the symmetrical model (i.e., α₁=0.5), which clearly favored the model with unequal cell contribution during the 1st cell generation (p<10⁻¹⁵). There is some evidence for asymmetrical contributions for later cell generations; however, since the asymmetric parameter α₁ estimated from the 2nd cell generation showed poor stability (FIG. 17C, p=0.004 compared to only one asymmetric cell division), asymmetric contribution was only assumed for the first cell generation. A 95% C.I. ([0.582, 0.607], FIG. 17D) was constructed using the likelihood ratio.

### Example 5: Ultra-Sensitive Rapid Detection and Validation of Low-Frequency Somatic Mutations

The triple-primer PCR sequencing method substantially increases the throughput and sensitivity for the detection and validation of somatic mutations (FIGS. 4 and 5). This method utilizes multiple unique, carefully designed, custom primers targeting a region of interest in the genome to identify a novel mutation or assess the alternate allele fraction (AAF) of a known mutation in one or more samples. Unlike existing methods such as ddPCR, triple-primer PCR sequencing often requires little to no optimization after primer design and is less sensitive to DNA source, concentration, and nucleotide context. The robust sensitivity of the method detects and validates somatic and germline mutations using the Ion Torrent S5 platform and detects of novel alleles through modifications for Illumina sequencing.

### Description of Triple Primer PCR Sequencing

While numerous studies have sought to define the error rates for the Ion Torrent platform due to the potential increased rate of insertion and deletion errors, particularly at homopolymers, the exact error rate appears to vary from sample to sample. Even more, while the rate of indel errors is likely elevated in the Ion Torrent platform over Illumina technology, the rates of SNV errors appear to be similar. It is likely that many estimates of errors are compounded by the combined effects of polymerase induced errors, mapping issues, and sequencing artifacts, all of which are known to reduce the sensitivity of detecting somatic mutations present in low fractions of a sample. Therefore, triple-primer PCR sequencing was developed to assess and partially mitigate these errors, while leveraging the rates to provide statistical confidence about a given mutation.

Prior studies have demonstrated the method of validating low AAF alleles using ultra-deep amplicon sequencing. However, technical issues including allelic dropout, artifacts (e.g., PCR- and sequencing platform-induced) and PCR duplicates can reduce the accuracy detected AAFs and possible result in both false negative calls as well as skewed AAFs. Triple-primer PCR sequencing overcomes these limitations through the use of multiple unique primers that are specifically designed to prevent sharing binding sites while avoiding known mutations (i.e., individual specific and general population) but are within 250 nucleotides (nts) of the target mutation. Once designed, unique primer-specific barcodes are appended to the reverse primers, along with Ion Torrent adapters. Optionally, Illumina adapters and/or 10 nt molecular barcodes can be appended to the primers to improve sensitivity or usage on the Illumina platform. Customized primers amplify targets including the mutation or region of interest using reduced cycling and minimal amounts of DNA, and amplification products are sequenced on either the Ion Torrent S5 or Illumina MiSeq platform for ultra-deep coverage. This optimized process allows for independent analyses of each primer pair, determination of error rates bases on amplicon-specific error rates (i.e., level of PCR and sequencing induced artifacts across the amplicon), identification of allelic imbalances from additional mutations affecting primer binding or chromatin structure, and the assessment of the variation in AAF among primers. Together, these steps provide a robust and low-cost strategy for extremely precise estimation of AAFs which is broadly applicable to studies of somatic and germline mutations.

### Accounting for Error Rates in Ion Torrent Data.

As the utility of the presently described invention relies on overcoming the previously described limitations of somatic mutation detection, triplicate unique primer sets were first designed around 5 known germline mutations (Tables 6A-6C) previously identified in bulk genomic DNA for testing the error rates of the method. The reduced PCR cycling conditions with a high-fidelity polymerase (4.4×10⁻⁷; Phusion HS, ThermoFisher) is estimated to result in an error rate of 8.8×10⁻⁶ at any given nucleotide position (ThermoFisher PCR Fidelity Calculator). Given that error rates vary amongst amplicons due to the specific nucleotide content of each amplicon, an internal control was designed for assigning the significance of each identified mutation. Using these primers, background error rates from PCR and sequencing, the sensitivity to detect extremely low AAFs, accuracy of the ascertained AAF measurement, and required DNA input and sequencing depths were assessed.

First, reads and nucleotides were stringently filtered for nucleotide and mapping qualities (q>20 and Q>20), resulting in the removal of an average of 10% of bases at any given nucleotide position. Relaxing these parameters (e.g., q10, Q10) did not decrease the fraction of excluded sites or assessed AAF, supporting that most nucleotide positions are of high quality. Next, the rate of artifacts in the region of the amplicon surrounding the mutation of interest was assessed by the AAF of all alternate alleles at each position under the assumption that all non-reference high-quality alleles present at sites not known to have a mutation represent errors. Across all amplicons, a low average background mutation frequency (0.018% AAF+/−0.0067%) was found for nucleotides located in the flanking 50 nt on either site of a mutation. Consistent with prior studies, some amplicons exhibited positional variability in error rates due to mapping errors around indels, including artifacts arising during sequencing.

To further reduce the rate of indel-associated errors, a computational modeling approach that detects and corrects sequencing platform errors was incorporated. Specifically, Pollux, a recent error modeling algorithm that screens for and corrects an estimated >95% of all indel associated errors, was used. The correction of indel-associated errors resulted in nearly a 5-fold reduction in nucleotide error frequency (0.0034%+/−0.0009%), allowing for mutations at extremely low AAFs to be distinguished from background sequencing and PCR-induced artifacts.

TABLE 6A

| | | | | | | Product | Product | | |
|---|---|---|---|---|---|---|---|---|---|
| Chromosome | AlleleStart | AlleleEnd | Ref | Alt | Gene | Start | end | InsertStart | InsertEnd |
| X | 153579431 | 153579431 | T | C | FLNA | 153579266 | 153579517 | 153579284 | 153579499 |
| X | 153579431 | 153579431 | T | C | FLNA | 153579289 | 153579555 | 153579311 | 153579536 |
| X | 153579431 | 153579431 | T | C | FLNA | 153579379 | 153579637 | 153579397 | 153579619 |
| 12 | 46321441 | 46321441 | T | G | SCAF11 | 46321317 | 46321542 | 46321343 | 46321517 |
| 12 | 46321441 | 46321441 | T | G | SCAF11 | 46321246 | 46321470 | 46321271 | 46321448 |
| 12 | 46321441 | 46321441 | T | G | SCAF11 | 46321376 | 46321606 | 46321399 | 46321585 |
| X | 153594210 | 153594210 | C | T | FLNA | 153593965 | 153594295 | 153593983 | 153594277 |
| X | 153594210 | 153594210 | C | T | FLNA | 153594163 | 153594424 | 153594181 | 153594406 |
| X | 153594210 | 153594210 | C | T | FLNA | 153594114 | 153594378 | 153594132 | 153594360 |
| 16 | 3639306 | 3639306 | G | A | SLX4 | 3639180 | 3639447 | 3639200 | 3639427 |
| 16 | 3639306 | 3639306 | G | A | SLX4 | 3639109 | 3639337 | 3639129 | 3639319 |
| 16 | 3639306 | 3639306 | G | A | SLX4 | 3639209 | 3639498 | 3639227 | 3639478 |
| X | 153599770 | 153599770 | G | T | FLNA | 153599611 | 153599868 | 153599629 | 153599850 |
| X | 153599770 | 153599770 | G | T | FLNA | 153599708 | 153599994 | 153599726 | 153599976 |
| X | 153599770 | 153599770 | G | T | FLNA | 153599747 | 153600008 | 153599766 | 153599989 |
| 18 | 21453038 | 21453038 | C | T | LAMA3 | 21452938 | 21453163 | 21452959 | 21453143 |
| 18 | 21453038 | 21453038 | C | T | LAMA3 | 21452848 | 21453097 | 21452867 | 21453076 |
| 18 | 21453038 | 21453038 | C | T | LAMA3 | 21453007 | 21453231 | 21453025 | 21453208 |
| X | 153587777 | 153587777 | G | C | FLNA | 153587660 | 153587885 | 153587682 | 153587865 |
| X | 153587777 | 153587777 | G | C | FLNA | 153587508 | 153587801 | 153587528 | 153587781 |
| X | 153587777 | 153587777 | G | C | FLNA | 153587606 | 153587897 | 153587626 | 153587878 |
| 19 | 44571260 | 44571260 | C | A | ZNF223 | 44571155 | 44571379 | 44571175 | 44571359 |
| 19 | 44571260 | 44571260 | C | A | ZNF223 | 44571066 | 44571291 | 44571085 | 44571270 |
| 19 | 44571260 | 44571260 | C | A | ZNF223 | 44571227 | 44571456 | 44571251 | 44571429 |
| X | 153579431 | 153579431 | T | C | FLNA | 153579266 | 153579517 | 153579284 | 153579499 |
| X | 153579431 | 153579431 | T | C | FLNA | 153579289 | 153579555 | 153579311 | 153579536 |
| X | 153579431 | 153579431 | T | C | FLNA | 153579379 | 153579637 | 153579397 | 153579619 |
| 12 | 46321441 | 46321441 | T | G | SCAF11 | 46321317 | 46321542 | 46321343 | 46321517 |
| 12 | 46321441 | 46321441 | T | G | SCAF11 | 46321246 | 46321470 | 46321271 | 46321448 |
| 12 | 46321441 | 46321441 | T | G | SCAF11 | 46321376 | 46321606 | 46321399 | 46321585 |
| X | 153594210 | 153594210 | C | T | FLNA | 153593965 | 153594295 | 153593983 | 153594277 |
| X | 153594210 | 153594210 | C | T | FLNA | 153594163 | 153594424 | 153594181 | 153594406 |
| X | 153594210 | 153594210 | C | T | FLNA | 153594114 | 153594378 | 153594132 | 153594360 |
| 16 | 3639306 | 3639306 | G | A | SLX4 | 3639180 | 3639447 | 3639200 | 3639427 |
| 16 | 3639306 | 3639306 | G | A | SLX4 | 3639109 | 3639337 | 3639129 | 3639319 |
| 16 | 3639306 | 3639306 | G | A | SLX4 | 3639209 | 3639498 | 3639227 | 3639478 |
| X | 153599770 | 153599770 | G | T | FLNA | 153599611 | 153599868 | 153599629 | 153599850 |
| X | 153599770 | 153599770 | G | T | FLNA | 153599708 | 153599994 | 153599726 | 153599976 |
| X | 153599770 | 153599770 | G | T | FLNA | 153599747 | 153600008 | 153599766 | 153599989 |
| 18 | 21453038 | 21453038 | C | T | LAMA3 | 21452938 | 21453163 | 21452959 | 21453143 |
| 18 | 21453038 | 21453038 | C | T | LAMA3 | 21452848 | 21453097 | 21452867 | 21453076 |
| 18 | 21453038 | 21453038 | C | T | LAMA3 | 21453007 | 21453231 | 21453025 | 21453208 |
| X | 153587777 | 153587777 | G | C | FLNA | 153587660 | 153587885 | 153587682 | 153587865 |
| X | 153587777 | 153587777 | G | C | FLNA | 153587508 | 153587801 | 153587528 | 153587781 |
| X | 153587777 | 153587777 | G | C | FLNA | 153587606 | 153587897 | 153587626 | 153587878 |
| 19 | 44571260 | 44571260 | C | A | ZNF223 | 44571155 | 44571379 | 44571175 | 44571359 |
| 19 | 44571260 | 44571260 | C | A | ZNF223 | 44571066 | 44571291 | 44571085 | 44571270 |
| 19 | 44571260 | 44571260 | C | A | ZNF223 | 44571227 | 44571456 | 44571251 | 44571429 |
| X | 153579431 | 153579431 | T | C | FLNA | 153579266 | 153579517 | 153579284 | 153579499 |
| X | 153579431 | 153579431 | T | C | FLNA | 153579289 | 153579555 | 153579311 | 153579536 |
| X | 153579431 | 153579431 | T | C | FLNA | 153579379 | 153579637 | 153579397 | 153579619 |
| 12 | 46321441 | 46321441 | T | G | SCAF11 | 46321317 | 46321542 | 46321343 | 46321517 |
| 12 | 46321441 | 46321441 | T | G | SCAF11 | 46321246 | 46321470 | 46321271 | 46321448 |
| 12 | 46321441 | 46321441 | T | G | SCAF11 | 46321376 | 46321606 | 46321399 | 46321585 |
| X | 153594210 | 153594210 | C | T | FLNA | 153593965 | 153594295 | 153593983 | 153594277 |
| X | 153594210 | 153594210 | C | T | FLNA | 153594163 | 153594424 | 153594181 | 153594406 |
| X | 153594210 | 153594210 | C | T | FLNA | 153594114 | 153594378 | 153594132 | 153594360 |
| 16 | 3639306 | 3639306 | G | A | SLX4 | 3639180 | 3639447 | 3639200 | 3639427 |
| 16 | 3639306 | 3639306 | G | A | SLX4 | 3639109 | 3639337 | 3639129 | 3639319 |
| 16 | 3639306 | 3639306 | G | A | SLX4 | 3639209 | 3639498 | 3639227 | 3639478 |
| X | 153599770 | 153599770 | G | T | FLNA | 153599611 | 153599868 | 153599629 | 153599850 |
| X | 153599770 | 153599770 | G | T | FLNA | 153599708 | 153599994 | 153599726 | 153599976 |
| X | 153599770 | 153599770 | G | T | FLNA | 153599747 | 153600008 | 153599766 | 153599989 |
| 18 | 21453038 | 21453038 | C | T | LAMA3 | 21452938 | 21453163 | 21452959 | 21453143 |
| 18 | 21453038 | 21453038 | C | T | LAMA3 | 21452848 | 21453097 | 21452867 | 21453076 |
| 18 | 21453038 | 21453038 | C | T | LAMA3 | 21453007 | 21453231 | 21453025 | 21453208 |
| X | 153587777 | 153587777 | G | C | FLNA | 153587660 | 153587885 | 153587682 | 153587865 |
| X | 153587777 | 153587777 | G | C | FLNA | 153587508 | 153587801 | 153587528 | 153587781 |
| X | 153587777 | 153587777 | G | C | FLNA | 153587606 | 153587897 | 153587626 | 153587878 |
| 19 | 44571260 | 44571260 | C | A | ZNF223 | 44571155 | 44571379 | 44571175 | 44571359 |
| 19 | 44571260 | 44571260 | C | A | ZNF223 | 44571066 | 44571291 | 44571085 | 44571270 |
| 19 | 44571260 | 44571260 | C | A | ZNF223 | 44571227 | 44571456 | 44571251 | 44571429 |
| X | 153579431 | 153579431 | T | C | FLNA | 153579266 | 153579517 | 153579284 | 153579499 |
| X | 153579431 | 153579431 | T | C | FLNA | 153579289 | 153579555 | 153579311 | 153579536 |
| X | 153579431 | 153579431 | T | C | FLNA | 153579379 | 153579637 | 153579397 | 153579619 |
| 12 | 46321441 | 46321441 | T | G | SCAF11 | 46321317 | 46321542 | 46321343 | 46321517 |
| 12 | 46321441 | 46321441 | T | G | SCAF11 | 46321246 | 46321470 | 46321271 | 46321448 |
| 12 | 46321441 | 46321441 | T | G | SCAF11 | 46321376 | 46321606 | 46321399 | 46321585 |
| X | 153594210 | 153594210 | C | T | FLNA | 153593965 | 153594295 | 153593983 | 153594277 |
| X | 153594210 | 153594210 | C | T | FLNA | 153594163 | 153594424 | 153594181 | 153594406 |
| X | 153594210 | 153594210 | C | T | FLNA | 153594114 | 153594378 | 153594132 | 153594360 |
| 16 | 3639306 | 3639306 | G | A | SLX4 | 3639180 | 3639447 | 3639200 | 3639427 |
| 16 | 3639306 | 3639306 | G | A | SLX4 | 3639109 | 3639337 | 3639129 | 3639319 |
| 16 | 3639306 | 3639306 | G | A | SLX4 | 3639209 | 3639498 | 3639227 | 3639478 |
| X | 153599770 | 153599770 | G | T | FLNA | 153599611 | 153599868 | 153599629 | 153599850 |
| X | 153599770 | 153599770 | G | T | FLNA | 153599708 | 153599994 | 153599726 | 153599976 |
| X | 153599770 | 153599770 | G | T | FLNA | 153599747 | 153600008 | 153599766 | 153599989 |
| 18 | 21453038 | 21453038 | C | T | LAMA3 | 21452938 | 21453163 | 21452959 | 21453143 |
| 18 | 21453038 | 21453038 | C | T | LAMA3 | 21452848 | 21453097 | 21452867 | 21453076 |
| 18 | 21453038 | 21453038 | C | T | LAMA3 | 21453007 | 21453231 | 21453025 | 21453208 |
| X | 153587777 | 153587777 | G | C | FLNA | 153587660 | 153587885 | 153587682 | 153587865 |
| X | 153587777 | 153587777 | G | C | FLNA | 153587508 | 153587801 | 153587528 | 153587781 |
| X | 153587777 | 153587777 | G | C | FLNA | 153587606 | 153587897 | 153587626 | 153587878 |
| 19 | 44571260 | 44571260 | C | A | ZNF223 | 44571155 | 44571379 | 44571175 | 44571359 |
| 19 | 44571260 | 44571260 | C | A | ZNF223 | 44571066 | 44571291 | 44571085 | 44571270 |
| 19 | 44571260 | 44571260 | C | A | ZNF223 | 44571227 | 44571456 | 44571251 | 44571429 |
| X | 153579431 | 153579431 | T | C | FLNA | 153579266 | 153579517 | 153579284 | 153579499 |
| X | 153579431 | 153579431 | T | C | FLNA | 153579289 | 153579555 | 153579311 | 153579536 |
| X | 153579431 | 153579431 | T | C | FLNA | 153579379 | 153579637 | 153579397 | 153579619 |
| 12 | 46321441 | 46321441 | T | G | SCAF11 | 46321317 | 46321542 | 46321343 | 46321517 |
| 12 | 46321441 | 46321441 | T | G | SCAF11 | 46321246 | 46321470 | 46321271 | 46321448 |
| 12 | 46321441 | 46321441 | T | G | SCAF11 | 46321376 | 46321606 | 46321399 | 46321585 |
| X | 153594210 | 153594210 | C | T | FLNA | 153593965 | 153594295 | 153593983 | 153594277 |
| X | 153594210 | 153594210 | C | T | FLNA | 153594163 | 153594424 | 153594181 | 153594406 |
| X | 153594210 | 153594210 | C | T | FLNA | 153594114 | 153594378 | 153594132 | 153594360 |
| 16 | 3639306 | 3639306 | G | A | SLX4 | 3639180 | 3639447 | 3639200 | 3639427 |
| 16 | 3639306 | 3639306 | G | A | SLX4 | 3639109 | 3639337 | 3639129 | 3639319 |
| 16 | 3639306 | 3639306 | G | A | SLX4 | 3639209 | 3639498 | 3639227 | 3639478 |
| X | 153599770 | 153599770 | G | T | FLNA | 153599611 | 153599868 | 153599629 | 153599850 |
| X | 153599770 | 153599770 | G | T | FLNA | 153599708 | 153599994 | 153599726 | 153599976 |
| X | 153599770 | 153599770 | G | T | FLNA | 153599747 | 153600008 | 153599766 | 153599989 |
| 18 | 21453038 | 21453038 | C | T | LAMA3 | 21452938 | 21453163 | 21452959 | 21453143 |
| 18 | 21453038 | 21453038 | C | T | LAMA3 | 21452848 | 21453097 | 21452867 | 21453076 |
| 18 | 21453038 | 21453038 | C | T | LAMA3 | 21453007 | 21453231 | 21453025 | 21453208 |
| X | 153587777 | 153587777 | G | C | FLNA | 153587660 | 153587885 | 153587682 | 153587865 |
| X | 153587777 | 153587777 | G | C | FLNA | 153587508 | 153587801 | 153587528 | 153587781 |
| X | 153587777 | 153587777 | G | C | FLNA | 153587606 | 153587897 | 153587626 | 153587878 |
| 19 | 44571260 | 44571260 | C | A | ZNF223 | 44571155 | 44571379 | 44571175 | 44571359 |
| 19 | 44571260 | 44571260 | C | A | ZNF223 | 44571066 | 44571291 | 44571085 | 44571270 |
| 19 | 44571260 | 44571260 | C | A | ZNF223 | 44571227 | 44571456 | 44571251 | 44571429 |
| X | 153579431 | 153579431 | T | C | FLNA | 153579266 | 153579517 | 153579284 | 153579499 |
| X | 153579431 | 153579431 | T | C | FLNA | 153579289 | 153579555 | 153579311 | 153579536 |
| X | 153579431 | 153579431 | T | C | FLNA | 153579379 | 153579637 | 153579397 | 153579619 |
| 12 | 46321441 | 46321441 | T | G | SCAF11 | 46321317 | 46321542 | 46321343 | 46321517 |
| 12 | 46321441 | 46321441 | T | G | SCAF11 | 46321246 | 46321470 | 46321271 | 46321448 |
| 12 | 46321441 | 46321441 | T | G | SCAF11 | 46321376 | 46321606 | 46321399 | 46321585 |
| X | 153594210 | 153594210 | C | T | FLNA | 153593965 | 153594295 | 153593983 | 153594277 |
| X | 153594210 | 153594210 | C | T | FLNA | 153594163 | 153594424 | 153594181 | 153594406 |
| X | 153594210 | 153594210 | C | T | FLNA | 153594114 | 153594378 | 153594132 | 153594360 |
| 16 | 3639306 | 3639306 | G | A | SLX4 | 3639180 | 3639447 | 3639200 | 3639427 |
| 16 | 3639306 | 3639306 | G | A | SLX4 | 3639109 | 3639337 | 3639129 | 3639319 |
| 16 | 3639306 | 3639306 | G | A | SLX4 | 3639209 | 3639498 | 3639227 | 3639478 |
| X | 153599770 | 153599770 | G | T | FLNA | 153599611 | 153599868 | 153599629 | 153599850 |
| X | 153599770 | 153599770 | G | T | FLNA | 153599708 | 153599994 | 153599726 | 153599976 |
| X | 153599770 | 153599770 | G | T | FLNA | 153599747 | 153600008 | 153599766 | 153599989 |
| 18 | 21453038 | 21453038 | C | T | LAMA3 | 21452938 | 21453163 | 21452959 | 21453143 |
| 18 | 21453038 | 21453038 | C | T | LAMA3 | 21452848 | 21453097 | 21452867 | 21453076 |
| 18 | 21453038 | 21453038 | C | T | LAMA3 | 21453007 | 21453231 | 21453025 | 21453208 |
| X | 153587777 | 153587777 | G | C | FLNA | 153587660 | 153587885 | 153587682 | 153587865 |
| X | 153587777 | 153587777 | G | C | FLNA | 153587508 | 153587801 | 153587528 | 153587781 |
| X | 153587777 | 153587777 | G | C | FLNA | 153587606 | 153587897 | 153587626 | 153587878 |
| 19 | 44571260 | 44571260 | C | A | ZNF223 | 44571155 | 44571379 | 44571175 | 44571359 |
| 19 | 44571260 | 44571260 | C | A | ZNF223 | 44571066 | 44571291 | 44571085 | 44571270 |
| 19 | 44571260 | 44571260 | C | A | ZNF223 | 44571227 | 44571456 | 44571251 | 44571429 |

TABLE 6B

| Chromosome | AlleleStart | AlleleEnd | Forward |
|---|---|---|---|
| X | 153579431 | 153579431 | CCTCTCTATGGGCAGTCGGTGATCAGGGCCTCACCTTGGTC |
| X | 153579431 | 153579431 | CCTCTCTATGGGCAGTCGGTGATCTGTGACATAGCACTCCTCCAG |
| X | 153579431 | 153579431 | CCTCTCTATGGGCAGTCGGTGATAGGCTGGCTGGTTGACCT |
| 12 | 46321441 | 46321441 | CCTCTCTATGGGCAGTCGGTGATAATCACACTCCATAGGTATCATTTCA |
| 12 | 46321441 | 46321441 | CCTCTCTATGGGCAGTCGGTGATTTCATTCATTTGTTTAAGATCAGCA |
| 12 | 46321441 | 46321441 | CCTCTCTATGGGCAGTCGGTGATTCAATGTGTGTTTTAGGCAACTC |
| X | 153594210 | 153594210 | CCTCTCTATGGGCAGTCGGTGATAGGGGGACATGCAAGACA |
| X | 153594210 | 153594210 | CCTCTCTATGGGCAGTCGGTGATGCGAGCTCTTCCGAAGGT |
| X | 153594210 | 153594210 | CCTCTCTATGGGCAGTCGGTGATGTTGACCCTGTGGGCAGA |
| 16 | 3639306 | 3639306 | CCTCTCTATGGGCAGTCGGTGATTCCTCTGGGTAGTGCAGCTT |
| 16 | 3639306 | 3639306 | CCTCTCTATGGGCAGTCGGTGATCAGAGCCGAATTCAGAAAGC |
| 16 | 3639306 | 3639306 | CCTCTCTATGGGCAGTCGGTGATGGGGTGGTGTCCAGGAGT |
| X | 153599770 | 153599770 | CCTCTCTATGGGCAGTCGGTGATCATTTTGAGGCGCGAGAA |
| X | 153599770 | 153599770 | CCTCTCTATGGGCAGTCGGTGATGAGGCAGGGAGCAGAGGT |
| X | 153599770 | 153599770 | CCTCTCTATGGGCAGTCGGTGATCCTTTAAATGCGGGAGGAG |
| 18 | 21453038 | 21453038 | CCTCTCTATGGGCAGTCGGTGATGAGCAGGAAGGGCAGGTATAA |
| 18 | 21453038 | 21453038 | CCTCTCTATGGGCAGTCGGTGATCTGGCACAGGCTGACTCAT |
| 18 | 21453038 | 21453038 | CCTCTCTATGGGCAGTCGGTGATGGATGCCTCCAGCAGTGA |
| X | 153587777 | 153587777 | CCTCTCTATGGGCAGTCGGTGATAGCCTCATAAGGGATGTACTCG |
| X | 153587777 | 153587777 | CCTCTCTATGGGCAGTCGGTGATCCTTGAAAGGACTGCCTGAG |
| X | 153587777 | 153587777 | CCTCTCTATGGGCAGTCGGTGATCTCCTCACCTGGCACTTGAT |
| 19 | 44571260 | 44571260 | CCTCTCTATGGGCAGTCGGTGATAGAGCCCACACAGGAGAGAG |
| 19 | 44571260 | 44571260 | CCTCTCTATGGGCAGTCGGTGATATCAGCGAGTCCACACTGG |
| 19 | 44571260 | 44571260 | CCTCTCTATGGGCAGTCGGTGATTTGAATCATAAGAGACTCCATTGC |
| X | 153579431 | 153579431 | CCTCTCTATGGGCAGTCGGTGATCAGGGCCTCACCTTGGTC |
| X | 153579431 | 153579431 | CCTCTCTATGGGCAGTCGGTGATCTGTGACATAGCACTCCTCCAG |
| X | 153579431 | 153579431 | CCTCTCTATGGGCAGTCGGTGATAGGCTGGCTGGTTGACCT |
| 12 | 46321441 | 46321441 | CCTCTCTATGGGCAGTCGGTGATAATCACACTCCATAGGTATCATTTCA |
| 12 | 46321441 | 46321441 | CCTCTCTATGGGCAGTCGGTGATTTCATTCATTTGTTTAAGATCAGCA |
| 12 | 46321441 | 46321441 | CCTCTCTATGGGCAGTCGGTGATTCAATGTGTGTTTTAGGCAACTC |
| X | 153594210 | 153594210 | CCTCTCTATGGGCAGTCGGTGATAGGGGGACATGCAAGACA |
| X | 153594210 | 153594210 | CCTCTCTATGGGCAGTCGGTGATGCGAGCTCTTCCGAAGGT |
| X | 153594210 | 153594210 | CCTCTCTATGGGCAGTCGGTGATGTTGACCCTGTGGGCAGA |
| 16 | 3639306 | 3639306 | CCTCTCTATGGGCAGTCGGTGATTCCTCTGGGTAGTGCAGCTT |
| 16 | 3639306 | 3639306 | CCTCTCTATGGGCAGTCGGTGATCAGAGCCGAATTCAGAAAGC |
| 16 | 3639306 | 3639306 | CCTCTCTATGGGCAGTCGGTGATGGGGTGGTGTCCAGGAGT |
| X | 153599770 | 153599770 | CCTCTCTATGGGCAGTCGGTGATCATTTTGAGGCGCGAGAA |
| X | 153599770 | 153599770 | CCTCTCTATGGGCAGTCGGTGATGAGGCAGGGAGCAGAGGT |
| X | 153599770 | 153599770 | CCTCTCTATGGGCAGTCGGTGATCCTTTAAATGCGGGAGGAG |
| 18 | 21453038 | 21453038 | CCTCTCTATGGGCAGTCGGTGATGAGCAGGAAGGGCAGGTATAA |
| 18 | 21453038 | 21453038 | CCTCTCTATGGGCAGTCGGTGATCTGGCACAGGCTGACTCAT |
| 18 | 21453038 | 21453038 | CCTCTCTATGGGCAGTCGGTGATGGATGCCTCCAGCAGTGA |
| X | 153587777 | 153587777 | CCTCTCTATGGGCAGTCGGTGATAGCCTCATAAGGGATGTACTCG |
| X | 153587777 | 153587777 | CCTCTCTATGGGCAGTCGGTGATCCTTGAAAGGACTGCCTGAG |
| X | 153587777 | 153587777 | CCTCTCTATGGGCAGTCGGTGATCTCCTCACCTGGCACTTGAT |
| 19 | 44571260 | 44571260 | CCTCTCTATGGGCAGTCGGTGATAGAGCCCACACAGGAGAGAG |
| 19 | 44571260 | 44571260 | CCTCTCTATGGGCAGTCGGTGATATCAGCGAGTCCACACTGG |
| 19 | 44571260 | 44571260 | CCTCTCTATGGGCAGTCGGTGATTTGAATCATAAGAGACTCCATTGC |
| X | 153579431 | 153579431 | CCTCTCTATGGGCAGTCGGTGATCAGGGCCTCACCTTGGTC |
| X | 153579431 | 153579431 | CCTCTCTATGGGCAGTCGGTGATCTGTGACATAGCACTCCTCCAG |
| X | 153579431 | 153579431 | CCTCTCTATGGGCAGTCGGTGATAGGCTGGCTGGTTGACCT |
| 12 | 46321441 | 46321441 | CCTCTCTATGGGCAGTCGGTGATAATCACACTCCATAGGTATCATTTCA |
| 12 | 46321441 | 46321441 | CCTCTCTATGGGCAGTCGGTGATTTCATTCATTTGTTTAAGATCAGCA |
| 12 | 46321441 | 46321441 | CCTCTCTATGGGCAGTCGGTGATTCAATGTGTGTTTTAGGCAACTC |
| X | 153594210 | 153594210 | CCTCTCTATGGGCAGTCGGTGATAGGGGGACATGCAAGACA |
| X | 153594210 | 153594210 | CCTCTCTATGGGCAGTCGGTGATGCGAGCTCTTCCGAAGGT |
| X | 153594210 | 153594210 | CCTCTCTATGGGCAGTCGGTGATGTTGACCCTGTGGGCAGA |
| 16 | 3639306 | 3639306 | CCTCTCTATGGGCAGTCGGTGATTCCTCTGGGTAGTGCAGCTT |
| 16 | 3639306 | 3639306 | CCTCTCTATGGGCAGTCGGTGATCAGAGCCGAATTCAGAAAGC |
| 16 | 3639306 | 3639306 | CCTCTCTATGGGCAGTCGGTGATGGGGTGGTGTCCAGGAGT |
| X | 153599770 | 153599770 | CCTCTCTATGGGCAGTCGGTGATCATTTTGAGGCGCGAGAA |
| X | 153599770 | 153599770 | CCTCTCTATGGGCAGTCGGTGATGAGGCAGGGAGCAGAGGT |
| X | 153599770 | 153599770 | CCTCTCTATGGGCAGTCGGTGATCCTTTAAATGCGGGAGGAG |
| 18 | 21453038 | 21453038 | CCTCTCTATGGGCAGTCGGTGATGAGCAGGAAGGGCAGGTATAA |
| 18 | 21453038 | 21453038 | CCTCTCTATGGGCAGTCGGTGATCTGGCACAGGCTGACTCAT |
| 18 | 21453038 | 21453038 | CCTCTCTATGGGCAGTCGGTGATGGATGCCTCCAGCAGTGA |
| X | 153587777 | 153587777 | CCTCTCTATGGGCAGTCGGTGATAGCCTCATAAGGGATGTACTCG |
| X | 153587777 | 153587777 | CCTCTCTATGGGCAGTCGGTGATCCTTGAAAGGACTGCCTGAG |
| X | 153587777 | 153587777 | CCTCTCTATGGGCAGTCGGTGATCTCCTCACCTGGCACTTGAT |
| 19 | 44571260 | 44571260 | CCTCTCTATGGGCAGTCGGTGATAGAGCCCACACAGGAGAGAG |
| 19 | 44571260 | 44571260 | CCTCTCTATGGGCAGTCGGTGATATCAGCGAGTCCACACTGG |
| 19 | 44571260 | 44571260 | CCTCTCTATGGGCAGTCGGTGATTTGAATCATAAGAGACTCCATTGC |
| X | 153579431 | 153579431 | CCTCTCTATGGGCAGTCGGTGATCAGGGCCTCACCTTGGTC |
| X | 153579431 | 153579431 | CCTCTCTATGGGCAGTCGGTGATCTGTGACATAGCACTCCTCCAG |
| X | 153579431 | 153579431 | CCTCTCTATGGGCAGTCGGTGATAGGCTGGCTGGTTGACCT |
| 12 | 46321441 | 46321441 | CCTCTCTATGGGCAGTCGGTGATAATCACACTCCATAGGTATCATTTCA |
| 12 | 46321441 | 46321441 | CCTCTCTATGGGCAGTCGGTGATTTCATTCATTTGTTTAAGATCAGCA |
| 12 | 46321441 | 46321441 | CCTCTCTATGGGCAGTCGGTGATTCAATGTGTGTTTTAGGCAACTC |
| X | 153594210 | 153594210 | CCTCTCTATGGGCAGTCGGTGATAGGGGGACATGCAAGACA |
| X | 153594210 | 153594210 | CCTCTCTATGGGCAGTCGGTGATGCGAGCTCTTCCGAAGGT |
| X | 153594210 | 153594210 | CCTCTCTATGGGCAGTCGGTGATGTTGACCCTGTGGGCAGA |
| 16 | 3639306 | 3639306 | CCTCTCTATGGGCAGTCGGTGATTCCTCTGGGTAGTGCAGCTT |
| 16 | 3639306 | 3639306 | CCTCTCTATGGGCAGTCGGTGATCAGAGCCGAATTCAGAAAGC |
| 16 | 3639306 | 3639306 | CCTCTCTATGGGCAGTCGGTGATGGGGTGGTGTCCAGGAGT |
| X | 153599770 | 153599770 | CCTCTCTATGGGCAGTCGGTGATCATTTTGAGGCGCGAGAA |
| X | 153599770 | 153599770 | CCTCTCTATGGGCAGTCGGTGATGAGGCAGGGAGCAGAGGT |
| X | 153599770 | 153599770 | CCTCTCTATGGGCAGTCGGTGATCCTTTAAATGCGGGAGGAG |
| 18 | 21453038 | 21453038 | CCTCTCTATGGGCAGTCGGTGATGAGCAGGAAGGGCAGGTATAA |
| 18 | 21453038 | 21453038 | CCTCTCTATGGGCAGTCGGTGATCTGGCACAGGCTGACTCAT |
| 18 | 21453038 | 21453038 | CCTCTCTATGGGCAGTCGGTGATGGATGCCTCCAGCAGTGA |
| X | 153587777 | 153587777 | CCTCTCTATGGGCAGTCGGTGATAGCCTCATAAGGGATGTACTCG |
| X | 153587777 | 153587777 | CCTCTCTATGGGCAGTCGGTGATCCTTGAAAGGACTGCCTGAG |
| X | 153587777 | 153587777 | CCTCTCTATGGGCAGTCGGTGATCTCCTCACCTGGCACTTGAT |
| 19 | 44571260 | 44571260 | CCTCTCTATGGGCAGTCGGTGATAGAGCCCACACAGGAGAGAG |
| 19 | 44571260 | 44571260 | CCTCTCTATGGGCAGTCGGTGATATCAGCGAGTCCACACTGG |
| 19 | 44571260 | 44571260 | CCTCTCTATGGGCAGTCGGTGATTTGAATCATAAGAGACTCCATTGC |
| X | 153579431 | 153579431 | CCTCTCTATGGGCAGTCGGTGATCAGGGCCTCACCTTGGTC |
| X | 153579431 | 153579431 | CCTCTCTATGGGCAGTCGGTGATCTGTGACATAGCACTCCTCCAG |
| X | 153579431 | 153579431 | CCTCTCTATGGGCAGTCGGTGATAGGCTGGCTGGTTGACCT |
| 12 | 46321441 | 46321441 | CCTCTCTATGGGCAGTCGGTGATAATCACACTCCATAGGTATCATTTCA |
| 12 | 46321441 | 46321441 | CCTCTCTATGGGCAGTCGGTGATTTCATTCATTTGTTTAAGATCAGCA |
| 12 | 46321441 | 46321441 | CCTCTCTATGGGCAGTCGGTGATTCAATGTGTGTTTTAGGCAACTC |
| X | 153594210 | 153594210 | CCTCTCTATGGGCAGTCGGTGATAGGGGGACATGCAAGACA |
| X | 153594210 | 153594210 | CCTCTCTATGGGCAGTCGGTGATGCGAGCTCTTCCGAAGGT |
| X | 153594210 | 153594210 | CCTCTCTATGGGCAGTCGGTGATGTTGACCCTGTGGGCAGA |
| 16 | 3639306 | 3639306 | CCTCTCTATGGGCAGTCGGTGATTCCTCTGGGTAGTGCAGCTT |
| 16 | 3639306 | 3639306 | CCTCTCTATGGGCAGTCGGTGATCAGAGCCGAATTCAGAAAGC |
| 16 | 3639306 | 3639306 | CCTCTCTATGGGCAGTCGGTGATGGGGTGGTGTCCAGGAGT |
| X | 153599770 | 153599770 | CCTCTCTATGGGCAGTCGGTGATCATTTTGAGGCGCGAGAA |
| X | 153599770 | 153599770 | CCTCTCTATGGGCAGTCGGTGATGAGGCAGGGAGCAGAGGT |
| X | 153599770 | 153599770 | CCTCTCTATGGGCAGTCGGTGATCCTTTAAATGCGGGAGGAG |
| 18 | 21453038 | 21453038 | CCTCTCTATGGGCAGTCGGTGATGAGCAGGAAGGGCAGGTATAA |
| 18 | 21453038 | 21453038 | CCTCTCTATGGGCAGTCGGTGATCTGGCACAGGCTGACTCAT |
| 18 | 21453038 | 21453038 | CCTCTCTATGGGCAGTCGGTGATGGATGCCTCCAGCAGTGA |
| X | 153587777 | 153587777 | CCTCTCTATGGGCAGTCGGTGATAGCCTCATAAGGGATGTACTCG |
| X | 153587777 | 153587777 | CCTCTCTATGGGCAGTCGGTGATCCTTGAAAGGACTGCCTGAG |
| X | 153587777 | 153587777 | CCTCTCTATGGGCAGTCGGTGATCTCCTCACCTGGCACTTGAT |
| 19 | 44571260 | 44571260 | CCTCTCTATGGGCAGTCGGTGATAGAGCCCACACAGGAGAGAG |
| 19 | 44571260 | 44571260 | CCTCTCTATGGGCAGTCGGTGATATCAGCGAGTCCACACTGG |
| 19 | 44571260 | 44571260 | CCTCTCTATGGGCAGTCGGTGATTTGAATCATAAGAGACTCCATTGC |
| X | 153579431 | 153579431 | CCTCTCTATGGGCAGTCGGTGATCAGGGCCTCACCTTGGTC |
| X | 153579431 | 153579431 | CCTCTCTATGGGCAGTCGGTGATCTGTGACATAGCACTCCTCCAG |
| X | 153579431 | 153579431 | CCTCTCTATGGGCAGTCGGTGATAGGCTGGCTGGTTGACCT |
| 12 | 46321441 | 46321441 | CCTCTCTATGGGCAGTCGGTGATAATCACACTCCATAGGTATCATTTCA |
| 12 | 46321441 | 46321441 | CCTCTCTATGGGCAGTCGGTGATTTCATTCATTTGTTTAAGATCAGCA |
| 12 | 46321441 | 46321441 | CCTCTCTATGGGCAGTCGGTGATTCAATGTGTGTTTTAGGCAACTC |
| X | 153594210 | 153594210 | CCTCTCTATGGGCAGTCGGTGATAGGGGGACATGCAAGACA |
| X | 153594210 | 153594210 | CCTCTCTATGGGCAGTCGGTGATGCGAGCTCTTCCGAAGGT |
| X | 153594210 | 153594210 | CCTCTCTATGGGCAGTCGGTGATGTTGACCCTGTGGGCAGA |
| 16 | 3639306 | 3639306 | CCTCTCTATGGGCAGTCGGTGATTCCTCTGGGTAGTGCAGCTT |
| 16 | 3639306 | 3639306 | CCTCTCTATGGGCAGTCGGTGATCAGAGCCGAATTCAGAAAGC |
| 16 | 3639306 | 3639306 | CCTCTCTATGGGCAGTCGGTGATGGGGTGGTGTCCAGGAGT |
| X | 153599770 | 153599770 | CCTCTCTATGGGCAGTCGGTGATCATTTTGAGGCGCGAGAA |
| X | 153599770 | 153599770 | CCTCTCTATGGGCAGTCGGTGATGAGGCAGGGAGCAGAGGT |
| X | 153599770 | 153599770 | CCTCTCTATGGGCAGTCGGTGATCCTTTAAATGCGGGAGGAG |
| 18 | 21453038 | 21453038 | CCTCTCTATGGGCAGTCGGTGATGAGCAGGAAGGGCAGGTATAA |
| 18 | 21453038 | 21453038 | CCTCTCTATGGGCAGTCGGTGATCTGGCACAGGCTGACTCAT |
| 18 | 21453038 | 21453038 | CCTCTCTATGGGCAGTCGGTGATGGATGCCTCCAGCAGTGA |
| X | 153587777 | 153587777 | CCTCTCTATGGGCAGTCGGTGATAGCCTCATAAGGGATGTACTCG |
| X | 153587777 | 153587777 | CCTCTCTATGGGCAGTCGGTGATCCTTGAAAGGACTGCCTGAG |
| X | 153587777 | 153587777 | CCTCTCTATGGGCAGTCGGTGATCTCCTCACCTGGCACTTGAT |
| 19 | 44571260 | 44571260 | CCTCTCTATGGGCAGTCGGTGATAGAGCCCACACAGGAGAGAG |
| 19 | 44571260 | 44571260 | CCTCTCTATGGGCAGTCGGTGATATCAGCGAGTCCACACTGG |
| 19 | 44571260 | 44571260 | CCTCTCTATGGGCAGTCGGTGATTTGAATCATAAGAGACTCCATTGC |

TABLE 6C

| Chromosome | AlleleStart | AlleleEnd | Reverse | Barcode |
|---|---|---|---|---|
| X | 153579431 | 153579431 | CCATCTCATCCCTGCGTGTCTCCGACTCAGttaacggacgCGCCAGATGGGTAAGTGC | ttaacggacg |
| X | 153579431 | 153579431 | CCATCTCATCCCTGCGTGTCTCCGACTCAGtccggcttacTGCAAATCAGTGGCTCTCC | tccggcttac |
| X | 153579431 | 153579431 | CCATCTCATCCCTGCGTGTCTCCGACTCAGtctcattcagCTCCCTTCCTGCCACCTG | tctcattcag |
| 12 | 46321441 | 46321441 | CCATCTCATCCCTGCGTGTCTCCGACTCAGgcggtcatacACATGTGATACTTTTGGGAATGAA | gcggtcatac |
| | | | G | |
| 12 | 46321441 | 46321441 | CCATCTCATCCCTGCGTGTCTCCGACTCAGtaggacgttcCTTCTGAACACCAAATTGGAAA | taggacgttc |
| 12 | 46321441 | 46321441 | CCATCTCATCCCTGCGTGTCTCCGACTCAGacgacgcaacTGTTAAGAGCCCAGAGGTTCA | acgacgcaac |
| X | 153594210 | 153594210 | CCATCTCATCCCTGCGTGTCTCCGACTCAGcttctcggacGGGGCCCCTACTCTTTGA | cttctcggac |
| X | 153594210 | 153594210 | CCATCTCATCCCTGCGTGTCTCCGACTCAGcattgccgttCTCGCAGCCCCTACACTG | |
| X | 153594210 | 153594210 | CCATCTCATCCCTGCGTGTCTCCGACTCAGcgagccagaaTGACTGCCCTCTGCTGTG | cattgccgtta |
| 16 | 3639306 | 3639306 | CCATCTCATCCCTGCGTGTCTCCGACTCAGtgaggacggcAGTGACGATGAGCAGGAGGT | tgaggacggc |
| 16 | 3639306 | 3639306 | CCATCTCATCCCTGCGTGTCTCCGACTCAGgcctgcgcagGCCAATTCCCATTGACCA | gcctgcgcag |
| 16 | 3639306 | 3639306 | CCATCTCATCCCTGCGTGTCTCCGACTCAGgttgacgtctCCAAGCTTCCTGAACCAGAC | gttgacgtct |
| X | 153599770 | 153599770 | CCATCTCATCCCTGCGTGTCTCCGACTCAGgagatcgattCTAGTGGGGGCATTCCAA | gagatcgatt |
| X | 153599770 | 153599770 | CCATCTCATCCCTGCGTGTCTCCGACTCAGagttcgagccCTCTAGGGCGCGTTTCCT | agttcgagcc |
| X | 153599770 | 153599770 | CCATCTCATCCCTGCGTGTCTCCGACTCAGctcaggctcaTCAGCCTTTCCTCGCTCTA | ctcaggctca |
| 18 | 21453038 | 21453038 | CCATCTCATCCCTGCGTGTCTCCGACTCAGggcaatataaTCCACATAACTCGCTTGCAG | ggcaatataa |
| 18 | 21453038 | 21453038 | CCATCTCATCCCTGCGTGTCTCCGACTCAGggtactcatgGAACTGTAGCCCAGACACTGC | ggtactcatg |
| 18 | 21453038 | 21453038 | CCATCTCATCCCTGCGTGTCTCCGACTCAGtctggttcaaACAAAGCTGGAAACTCTTCCCTA | tctggttcaa |
| X | 153587777 | 153587777 | CCATCTCATCCCTGCGTGTCTCCGACTCAGgtcctataagCCAACAAGCCCAACAAGTTC | gtcctataag |
| X | 153587777 | 153587777 | CCATCTCATCCCTGCGTGTCTCCGACTCAGgtcagcctccGAATGACCGGCTGTCTGTTT | gtcagcctcc |
| X | 153587777 | 153587777 | CCATCTCATCCCTGCGTGTCTCCGACTCAGttcaagctcgAAAGTGGCACCACCAACAA | ttcaagctcg |
| 19 | 44571260 | 44571260 | CCATCTCATCCCTGCGTGTCTCCGACTCAGgtaccagcgcCTTGTAGCGCTTCCCACAGT | gtaccagcgc |
| 19 | 44571260 | 44571260 | CCATCTCATCCCTGCGTGTCTCCGACTCAGtcctattcggAGCTTCTTTCCACAATCCTCA | tcctattcgg |
| 19 | 44571260 | 44571260 | CCATCTCATCCCTGCGTGTCTCCGACTCAGgccagcgattCTGTACCCCATAAATATGTACAACA | gccagcgatt |
| | | | CT | |
| X | 153579431 | 153579431 | CCATCTCATCCCTGCGTGTCTCCGACTCAGacctagactgCGCCAGATGGGTAAGTGC | acctagactg |
| X | 153579431 | 153579431 | CCATCTCATCCCTGCGTGTCTCCGACTCAGactggttcgcTGCAAATCAGTGGCTCTCC | actggttcgc |
| X | 153579431 | 153579431 | CCATCTCATCCCTGCGTGTCTCCGACTCAGccatattaggCTCCCTTCCTGCCACCTG | ccatattagg |
| 12 | 46321441 | 46321441 | CCATCTCATCCCTGCGTGTCTCCGACTCAGgctcgtcagcACATGTGATACTTTTGGGAATGAA | gctcgtcagc |
| | | | G | |
| 12 | 46321441 | 46321441 | CCATCTCATCCCTGCGTGTCTCCGACTCAGcgtaatgacgCTTCTGAACACCAAATTGGAAA | cgtaatgacg |
| 12 | 46321441 | 46321441 | CCATCTCATCCCTGCGTGTCTCCGACTCAGccggcgctgaTGTTAAGAGCCCAGAGGTTCA | ccggcgctga |
| X | 153594210 | 153594210 | CCATCTCATCCCTGCGTGTCTCCGACTCAGcgcgaagataGGGGCCCCTACTCTTTGA | cgcgaagata |
| X | 153594210 | 153594210 | CCATCTCATCCCTGCGTGTCTCCGACTCAGgaaccgcagaCTCGCAGCCCCTACACTG | gaaccgcaga |
| X | 153594210 | 153594210 | CCATCTCATCCCTGCGTGTCTCCGACTCAGttggcagagaTGACTGCCCTCTGCTGTG | ttggcagaga |
| 16 | 3639306 | 3639306 | CCATCTCATCCCTGCGTGTCTCCGACTCAGgcatctctgcAGTGACGATGAGCAGGAGGT | gcatctctgc |
| 16 | 3639306 | 3639306 | CCATCTCATCCCTGCGTGTCTCCGACTCAGttggaccgcaGCCAATTCCCATTGACCA | ttggaccgca |
| 16 | 3639306 | 3639306 | CCATCTCATCCCTGCGTGTCTCCGACTCAGgcagaacgtcCCAAGCTTCCTGAACCAGAC | gcagaacgtc |
| X | 153599770 | 153599770 | CCATCTCATCCCTGCGTGTCTCCGACTCAGaacttcgagcCTAGTGGGGGCATTCCAA | aacttcgagc |
| X | 153599770 | 153599770 | CCATCTCATCCCTGCGTGTCTCCGACTCAGgctcctagagCTCTAGGGCGCGTTTCCT | gctcctagag |
| X | 153599770 | 153599770 | CCATCTCATCCCTGCGTGTCTCCGACTCAGtatctagcttTCAGCCTTTCCTCGCTCTA | tatctagctt |
| 18 | 21453038 | 21453038 | CCATCTCATCCCTGCGTGTCTCCGACTCAGgagtattggcTCCACATAACTCGCTTGCAG | gagtattggc |
| 18 | 21453038 | 21453038 | CCATCTCATCCCTGCGTGTCTCCGACTCAGcctgagctcaGAACTGTAGCCCAGACACTGC | cctgagctca |
| 18 | 21453038 | 21453038 | CCATCTCATCCCTGCGTGTCTCCGACTCAGcaggcgagtaACAAAGCTGGAAACTCTTCCCTA | caggcgagta |
| X | 153587777 | 153587777 | CCATCTCATCCCTGCGTGTCTCCGACTCAGgcaggcagagCCAACAAGCCCAACAAGTTC | gcaggcagag |
| X | 153587777 | 153587777 | CCATCTCATCCCTGCGTGTCTCCGACTCAGgcgtcgatacGAATGACCGGCTGTCTGTTT | gcgtcgatac |
| X | 153587777 | 153587777 | CCATCTCATCCCTGCGTGTCTCCGACTCAGcgatgattatAAAGTGGCACCACCAACAA | cgatgattat |
| 19 | 44571260 | 44571260 | CCATCTCATCCCTGCGTGTCTCCGACTCAGgacggctggcCTTGTAGCGCTTCCCACAGT | gacggctggc |
| 19 | 44571260 | 44571260 | CCATCTCATCCCTGCGTGTCTCCGACTCAGggagcctgagAGCTTCTTTCCACAATCCTCA | ggagcctgag |
| 19 | 44571260 | 44571260 | CCATCTCATCCCTGCGTGTCTCCGACTCAGcctgactgctCTGTACCCCATAAATATGTACAACA | cctgactgct |
| | | | CT | |
| X | 153579431 | 153579431 | CCATCTCATCCCTGCGTGTCTCCGACTCAGacggctgacgCGCCAGATGGGTAAGTGC | acggctgacg |
| X | 153579431 | 153579431 | CCATCTCATCCCTGCGTGTCTCCGACTCAGtaaccatagcTGCAAATCAGTGGCTCTCC | taaccatagc |
| X | 153579431 | 153579431 | CCATCTCATCCCTGCGTGTCTCCGACTCAGtcttgccttcCTCCCTTCCTGCCACCTG | tcttgccttc |
| 12 | 46321441 | 46321441 | CCATCTCATCCCTGCGTGTCTCCGACTCAGttcttagattACATGTGATACTTTTGGGAATGAAG | ttcttagatt |
| 12 | 46321441 | 46321441 | CCATCTCATCCCTGCGTGTCTCCGACTCAGtcatctcattCTTCTGAACACCAAATTGGAAA | tcatctcatt |
| 12 | 46321441 | 46321441 | CCATCTCATCCCTGCGTGTCTCCGACTCAGtctccgctcgTGTTAAGAGCCCAGAGGTTCA | tctccgctcg |
| X | 153594210 | 153594210 | CCATCTCATCCCTGCGTGTCTCCGACTCAGtgccatatgcGGGGCCCCTACTCTTTGA | tgccatatgc |
| X | 153594210 | 153594210 | CCATCTCATCCCTGCGTGTCTCCGACTCAGtaaggcctctCTCGCAGCCCCTACACTG | taaggcctct |
| X | 153594210 | 153594210 | CCATCTCATCCCTGCGTGTCTCCGACTCAGgagtaggccgTGACTGCCCTCTGCTGTG | gagtaggccg |
| 16 | 3639306 | 3639306 | CCATCTCATCCCTGCGTGTCTCCGACTCAGgcaataagctAGTGACGATGAGCAGGAGGT | gcaataagct |
| 16 | 3639306 | 3639306 | CCATCTCATCCCTGCGTGTCTCCGACTCAGggcgttgcaaGCCAATTCCCATTGACCA | ggcgttgcaa |
| 16 | 3639306 | 3639306 | CCATCTCATCCCTGCGTGTCTCCGACTCAGccaagaagcgCCAAGCTTCCTGAACCAGAC | ccaagaagcg |
| X | 153599770 | 153599770 | CCATCTCATCCCTGCGTGTCTCCGACTCAGggttacctcgCTAGTGGGGGCATTCCAA | ggttacctcg |
| X | 153599770 | 153599770 | CCATCTCATCCCTGCGTGTCTCCGACTCAGctccgccttaCTCTAGGGCGCGTTTCCT | ctccgcctta |
| X | 153599770 | 153599770 | CCATCTCATCCCTGCGTGTCTCCGACTCAGctccagagatTCAGCCTTTCCTCGCTCTA | ctccagagat |
| 18 | 21453038 | 21453038 | CCATCTCATCCCTGCGTGTCTCCGACTCAGgtcgaggtagTCCACATAACTCGCTTGCAG | gtcgaggtag |
| 18 | 21453038 | 21453038 | CCATCTCATCCCTGCGTGTCTCCGACTCAGtatggacctgGAACTGTAGCCCAGACACTGC | tatggacctg |
| 18 | 21453038 | 21453038 | CCATCTCATCCCTGCGTGTCTCCGACTCAGtacctgctagACAAAGCTGGAAACTCTTCCCTA | tacctgctag |
| X | 153587777 | 153587777 | CCATCTCATCCCTGCGTGTCTCCGACTCAGccgcgaccgaCCAACAAGCCCAACAAGTTC | ccgcgaccga |
| X | 153587777 | 153587777 | CCATCTCATCCCTGCGTGTCTCCGACTCAGgttgaacgttGAATGACCGGCTGTCTGTTT | gttgaacgtt |
| X | 153587777 | 153587777 | CCATCTCATCCCTGCGTGTCTCCGACTCAGtgccaacgcaAAAGTGGCACCACCAACAA | tgccaacgca |
| 19 | 44571260 | 44571260 | CCATCTCATCCCTGCGTGTCTCCGACTCAGggattgacctCTTGTAGCGCTTCCCACAGT | ggattgacct |
| 19 | 44571260 | 44571260 | CCATCTCATCCCTGCGTGTCTCCGACTCAGggacggattcAGCTTCTTTCCACAATCCTCA | ggacggattc |
| 19 | 44571260 | 44571260 | CCATCTCATCCCTGCGTGTCTCCGACTCAGtcctccgtcgCTGTACCCCATAAATATGTACAACA | tcctccgtcg |
| | | | CT | |
| X | 153579431 | 153579431 | CCATCTCATCCCTGCGTGTCTCCGACTCAGagttcatggtCGCCAGATGGGTAAGTGC | agttcatggt |
| X | 153579431 | 153579431 | CCATCTCATCCCTGCGTGTCTCCGACTCAGtatccattccTGCAAATCAGTGGCTCTCC | tatccattcc |
| X | 153579431 | 153579431 | CCATCTCATCCCTGCGTGTCTCCGACTCAGggagagcgcgCTCCCTTCCTGCCACCTG | ggagagcgcg |
| 12 | 46321441 | 46321441 | CCATCTCATCCCTGCGTGTCTCCGACTCAGcggaccttggACATGTGATACTTTTGGGAATGAA | cggaccttgg |
| | | | G | |
| 12 | 46321441 | 46321441 | CCATCTCATCCCTGCGTGTCTCCGACTCAGggcaatctccCTTCTGAACACCAAATTGGAAA | ggcaatctcc |
| 12 | 46321441 | 46321441 | CCATCTCATCCCTGCGTGTCTCCGACTCAGaggattgattTGTTAAGAGCCCAGAGGTTCA | aggattgatt |
| X | 153594210 | 153594210 | CCATCTCATCCCTGCGTGTCTCCGACTCAGgccgttgcctGGGGCCCCTACTCTTTGA | gccgttgcct |
| X | 153594210 | 153594210 | CCATCTCATCCCTGCGTGTCTCCGACTCAGaagtacgtcgCTCGCAGCCCCTACACTG | aagtacgtcg |
| X | 153594210 | 153594210 | CCATCTCATCCCTGCGTGTCTCCGACTCAGtggcttaaggTGACTGCCCTCTGCTGTG | tggcttaagg |
| 16 | 3639306 | 3639306 | CCATCTCATCCCTGCGTGTCTCCGACTCAGctcttccagaAGTGACGATGAGCAGGAGGT | ctcttccaga |
| 16 | 3639306 | 3639306 | CCATCTCATCCCTGCGTGTCTCCGACTCAGcgttcttcaaGCCAATTCCCATTGACCA | cgttcttcaa |
| 16 | 3639306 | 3639306 | CCATCTCATCCCTGCGTGTCTCCGACTCAGcaacggctgcCCAAGCTTCCTGAACCAGAC | caacggctgc |
| X | 153599770 | 153599770 | CCATCTCATCCCTGCGTGTCTCCGACTCAGgcaagtaaccCTAGTGGGGGCATTCCAA | gcaagtaacc |
| X | 153599770 | 153599770 | CCATCTCATCCCTGCGTGTCTCCGACTCAGgttcatagtcCTCTAGGGCGCGTTTCCT | gttcatagtc |
| X | 153599770 | 153599770 | CCATCTCATCCCTGCGTGTCTCCGACTCAGacggcgagccTCAGCCTTTCCTCGCTCTA | acggcgagcc |
| 18 | 21453038 | 21453038 | CCATCTCATCCCTGCGTGTCTCCGACTCAGgtatggtcggTCCACATAACTCGCTTGCAG | gtatggtcgg |
| 18 | 21453038 | 21453038 | CCATCTCATCCCTGCGTGTCTCCGACTCAGtcggttatccGAACTGTAGCCCAGACACTGC | tcggttatcc |
| 18 | 21453038 | 21453038 | CCATCTCATCCCTGCGTGTCTCCGACTCAGgcggtcgataACAAAGCTGGAAACTCTTCCCTA | gcggtcgata |
| X | 153587777 | 153587777 | CCATCTCATCCCTGCGTGTCTCCGACTCAGtcctcagtatCCAACAAGCCCAACAAGTTC | tcctcagtat |
| X | 153587777 | 153587777 | CCATCTCATCCCTGCGTGTCTCCGACTCAGaccgttcctgGAATGACCGGCTGTCTGTTT | accgttcctg |
| X | 153587777 | 153587777 | CCATCTCATCCCTGCGTGTCTCCGACTCAGgcctgctcttAAAGTGGCACCACCAACAA | gcctgctctt |
| 19 | 44571260 | 44571260 | CCATCTCATCCCTGCGTGTCTCCGACTCAGagcgtaaccaCTTGTAGCGCTTCCCACAGT | agcgtaacca |
| 19 | 44571260 | 44571260 | CCATCTCATCCCTGCGTGTCTCCGACTCAGttgcctgatgAGCTTCTTTCCACAATCCTCA | ttgcctgatg |
| 19 | 44571260 | 44571260 | CCATCTCATCCCTGCGTGTCTCCGACTCAGttattgatctCTGTACCCCATAAATATGTACAACA | ttattgatct |
| | | | CT | |
| X | 153579431 | 153579431 | CCATCTCATCCCTGCGTGTCTCCGACTCAGtacgctcggaCGCCAGATGGGTAAGTGC | tacgctcgga |
| X | 153579431 | 153579431 | CCATCTCATCCCTGCGTGTCTCCGACTCAGcaatccaaggTGCAAATCAGTGGCTCTCC | caatccaagg |
| X | 153579431 | 153579431 | CCATCTCATCCCTGCGTGTCTCCGACTCAGtcgtagctatCTCCCTTCCTGCCACCTG | tcgtagctat |
| 12 | 46321441 | 46321441 | CCATCTCATCCCTGCGTGTCTCCGACTCAGcgctcatcgcACATGTGATACTTTTGGGAATGAA | cgctcatcgc |
| | | | G | |
| 12 | 46321441 | 46321441 | CCATCTCATCCCTGCGTGTCTCCGACTCAGtccgttcattCTTCTGAACACCAAATTGGAAA | tccgttcatt |
| 12 | 46321441 | 46321441 | CCATCTCATCCCTGCGTGTCTCCGACTCAGcggccaggctTGTTAAGAGCCCAGAGGTTCA | cggccaggct |
| X | 153594210 | 153594210 | CCATCTCATCCCTGCGTGTCTCCGACTCAGcaacctatctGGGGCCCCTACTCTTTGA | caacctatct |
| X | 153594210 | 153594210 | CCATCTCATCCCTGCGTGTCTCCGACTCAGcgtaatctcaCTCGCAGCCCCTACACTG | cgtaatctca |
| X | 153594210 | 153594210 | CCATCTCATCCCTGCGTGTCTCCGACTCAGatatcgcgacTGACTGCCCTCTGCTGTG | atatcgcgac |
| 16 | 3639306 | 3639306 | CCATCTCATCCCTGCGTGTCTCCGACTCAGtcaatatctgAGTGACGATGAGCAGGAGGT | tcaatatctg |
| 16 | 3639306 | 3639306 | CCATCTCATCCCTGCGTGTCTCCGACTCAGatagagtataGCCAATTCCCATTGACCA | atagagtata |
| 16 | 3639306 | 3639306 | CCATCTCATCCCTGCGTGTCTCCGACTCAGgcaactagttCCAAGCTTCCTGAACCAGAC | gcaactagtt |
| X | 153599770 | 153599770 | CCATCTCATCCCTGCGTGTCTCCGACTCAGatctcgaatcCTAGTGGGGGCATTCCAA | atctcgaatc |
| X | 153599770 | 153599770 | CCATCTCATCCCTGCGTGTCTCCGACTCAGccaggagcgaCTCTAGGGCGCGTTTCCT | ccaggagcga |
| X | 153599770 | 153599770 | CCATCTCATCCCTGCGTGTCTCCGACTCAGatctccatcgTCAGCCTTTCCTCGCTCTA | atctccatcg |
| 18 | 21453038 | 21453038 | CCATCTCATCCCTGCGTGTCTCCGACTCAGttgacgagctTCCACATAACTCGCTTGCAG | ttgacgagct |
| 18 | 21453038 | 21453038 | CCATCTCATCCCTGCGTGTCTCCGACTCAGtactattaccGAACTGTAGCCCAGACACTGC | tactattacc |
| 18 | 21453038 | 21453038 | CCATCTCATCCCTGCGTGTCTCCGACTCAGcgtcctggacACAAAGCTGGAAACTCTTCCCTA | cgtcctggac |
| X | 153587777 | 153587777 | CCATCTCATCCCTGCGTGTCTCCGACTCAGctcggcgcttCCAACAAGCCCAACAAGTTC | ctcggcgctt |
| X | 153587777 | 153587777 | CCATCTCATCCCTGCGTGTCTCCGACTCAGgatacgtaagGAATGACCGGCTGTCTGTTT | gatacgtaag |
| X | 153587777 | 153587777 | CCATCTCATCCCTGCGTGTCTCCGACTCAGctcggattaaAAAGTGGCACCACCAACAA | ctcggattaa |
| 19 | 44571260 | 44571260 | CCATCTCATCCCTGCGTGTCTCCGACTCAGttggattcgtCTTGTAGCGCTTCCCACAGT | ttggattcgt |
| 19 | 44571260 | 44571260 | CCATCTCATCCCTGCGTGTCTCCGACTCAGccgtccgctaAGCTTCTTTCCACAATCCTCA | ccgtccgcta |
| 19 | 44571260 | 44571260 | CCATCTCATCCCTGCGTGTCTCCGACTCAGgcgattgcaaCTGTACCCCATAAATATGTACAAC | gcgattgcaa |
| | | | ACT | |
| X | 153579431 | 153579431 | CCATCTCATCCCTGCGTGTCTCCGACTCAGccatgcataaCGCCAGATGGGTAAGTGC | ccatgcataa |
| X | 153579431 | 153579431 | CCATCTCATCCCTGCGTGTCTCCGACTCAGtaattgcaatTGCAAATCAGTGGCTCTCC | taattgcaat |
| X | 153579431 | 153579431 | CCATCTCATCCCTGCGTGTCTCCGACTCAGacgactccaaCTCCCTTCCTGCCACCTG | acgactccaa |
| 12 | 46321441 | 46321441 | CCATCTCATCCCTGCGTGTCTCCGACTCAGatcatgcagaACATGTGATACTTTTGGGAATGAA | atcatgcaga |
| | | | G | |
| 12 | 46321441 | 46321441 | CCATCTCATCCCTGCGTGTCTCCGACTCAGaactcctaatCTTCTGAACACCAAATTGGAAA | aactcctaat |
| 12 | 46321441 | 46321441 | CCATCTCATCCCTGCGTGTCTCCGACTCAGggatattcgtTGTTAAGAGCCCAGAGGTTCA | ggatattcgt |
| X | 153594210 | 153594210 | CCATCTCATCCCTGCGTGTCTCCGACTCAGtcggatgactGGGGCCCCTACTCTTTGA | tcggatgact |
| X | 153594210 | 153594210 | CCATCTCATCCCTGCGTGTCTCCGACTCAGgacgcgcgagCTCGCAGCCCCTACACTG | gacgcgcgag |
| X | 153594210 | 153594210 | CCATCTCATCCCTGCGTGTCTCCGACTCAGgcctagacctTGACTGCCCTCTGCTGTG | gcctagacct |
| 16 | 3639306 | 3639306 | CCATCTCATCCCTGCGTGTCTCCGACTCAGgaccaggcgaAGTGACGATGAGCAGGAGGT | gaccaggcga |
| 16 | 3639306 | 3639306 | CCATCTCATCCCTGCGTGTCTCCGACTCAGgctctggcgtGCCAATTCCCATTGACCA | gctctggcgt |
| 16 | 3639306 | 3639306 | CCATCTCATCCCTGCGTGTCTCCGACTCAGtggtccggaaCCAAGCTTCCTGAACCAGAC | tggtccggaa |
| X | 153599770 | 153599770 | CCATCTCATCCCTGCGTGTCTCCGACTCAGctctgcgtctCTAGTGGGGGCATTCCAA | ctctgcgtct |
| X | 153599770 | 153599770 | CCATCTCATCCCTGCGTGTCTCCGACTCAGccagaagcagCTCTAGGGCGCGTTTCCT | ccagaagcag |
| X | 153599770 | 153599770 | CCATCTCATCCCTGCGTGTCTCCGACTCAGggaaggttgcTCAGCCTTTCCTCGCTCTA | ggaaggttgc |
| 18 | 21453038 | 21453038 | CCATCTCATCCCTGCGTGTCTCCGACTCAGtaacggtacgTCCACATAACTCGCTTGCAG | taacggtacg |
| 18 | 21453038 | 21453038 | CCATCTCATCCCTGCGTGTCTCCGACTCAGctcgctcatgGAACTGTAGCCCAGACACTGC | ctcgctcatg |
| 18 | 21453038 | 21453038 | CCATCTCATCCCTGCGTGTCTCCGACTCAGactccaaggcACAAAGCTGGAAACTCTTCCCTA | actccaaggc |
| X | 153587777 | 153587777 | CCATCTCATCCCTGCGTGTCTCCGACTCAGgagctgctatCCAACAAGCCCAACAAGTTC | gagctgctat |
| X | 153587777 | 153587777 | CCATCTCATCCCTGCGTGTCTCCGACTCAGcgttgaggccGAATGACCGGCTGTCTGTTT | cgttgaggcc |
| X | 153587777 | 153587777 | CCATCTCATCCCTGCGTGTCTCCGACTCAGttctggatccAAAGTGGCACCACCAACAA | ttctggatcc |
| 19 | 44571260 | 44571260 | CCATCTCATCCCTGCGTGTCTCCGACTCAGccggattccaCTTGTAGCGCTTCCCACAGT | ccggattcca |
| 19 | 44571260 | 44571260 | CCATCTCATCCCTGCGTGTCTCCGACTCAGtccatcgcttAGCTTCTTTCCACAATCCTCA | tccatcgctt |
| 19 | 44571260 | 44571260 | CCATCTCATCCCTGCGTGTCTCCGACTCAGttacttctcaCTGTACCCCATAAATATGTACAACA | ttacttctca |
| | | | CT | |

### Sensitivity and Reproducibility of Assay

The AAF of somatic mutations can vary dramatically across tissues, where they can be nearly undetectable in tissues such as blood, but higher frequency in tissues like the brain. Given that most genetic testing is performed on blood or cell free DNA samples with anticipated low AAFs, the ability of the presently described methods to accurately detect AAFs at extremely low levels, which are often difficult or impossible to accurately assess by other methods.

The sensitivity of triple-primer PCR sequencing was assessed through serial dilution of a genomic control DNA sample containing the same 5 known germline mutations described above (Tables 6A-6C) with a control DNA lacking these mutations, thereby generating AAFs ranging from 50% down to 0.01%. The dilutions were amplified with primers for each mutation and sequenced on the Ion Torrent S5 with sequencing reads of 400 bp in length. All reads were processed using custom analytical scripts (described in methods), allowing the comparison of assessed and expected allelic fractions.

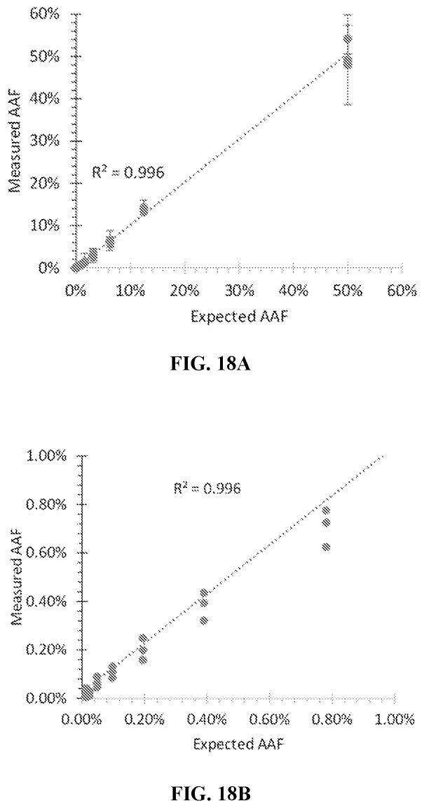

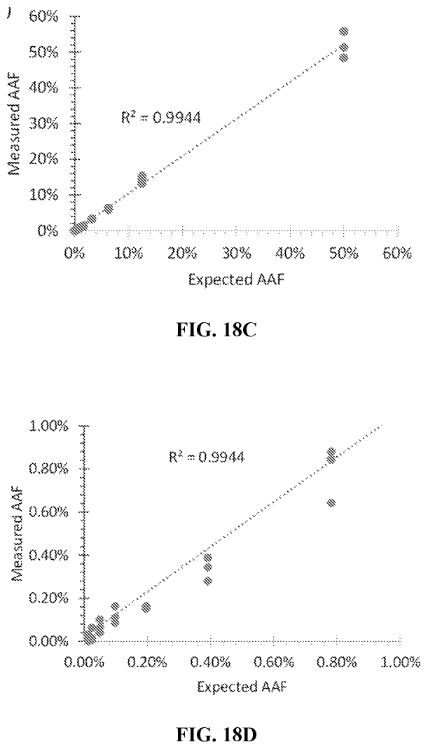

The presently described method accurately measures AAFs as low as 0.01% when using a 50 ng of genomic DNA, although for significant detection above the amplicon-specific error rates, AAFs were typically required to be above 0.05% (FIGS. 18A, 18B). Surprisingly, 6 of 6 mutations were successfully identified at AAFs of 0.05%, and all were identified by at least one of the primers in the sets at AAFs as low as 0.01%. Therefore, the presently described approach is able to achieve a 100% sensitivity for detection of alleles down to 0.01% AAF (FIGS. 18A, 18B). The largest factors observed in accurately measuring the AAFs at extremely low levels of below 0.05% was providing sufficient input DNA and achieving enough sequencing depth to distinguish errors from true calls. In this case, a depth of more than 50,000× is recommended for the best sensitivity. While each independent primer set can produce slightly different AAFs due to both inherent primer characteristics and variability amongst reactions, averaging the primers provides an extremely accurate assessment of the true AAF. Even more, the accuracy of the estimate is better assessed through the comparison of the confidence intervals from the AAFs of the mutation and the background error rates. For example, it was found that the measurement of a 2048-fold dilution (estimated AAF˜0.012%) sample resulted in an AAF of 0.0136%±0.012% while the background error rate was significantly lower that the measured AAF at 0.0015%±0.009%.

The measured AAFs (average across triple primer sets) were linearly correlated with the expected AAFs down to 0.01% (R²>0.999), though as expected, individual AAFs do vary amongst individual primers (R²>0.98). Therefore, while individual primer sets are prone to biases in AAFs, the utility of multiple primer provides a robust and accurate measurement.

DNA is often limited, particularly in clinical contexts, but is also known as an important factor for sensitivity for somatic alleles due to the presence of fewer DNA fragments containing the targeted allele. Therefore, the sensitivity of using 50 ng was compared to using a reduced concentration of 25 ng (˜3800 cells) (PMID: 30813969). With 3800 cells, the accurate detection of the lowest dilution of 0.01% AAF is unlikely as it would likely only be represented by a single fragment. Surprisingly, AAFs down to 0.05% remained detectable with 25 ng DNA (FIGS. 18C, 18D), though with less precision, which indicates that increasing the input DNA to 50 ng or more would improve accuracy when validating alleles below 0.1% AAF.

Furthermore, the impact of total sequencing depth on the accuracy was assessed to identify the minimum depth needed for accurate determination of AAFs. Sequencing data for each amplicon were randomly sampled to create artificial datasets containing a wide range of depths ranging from 10,000 to 150,000× coverage. Increasing read depths above 10,000× did not have a substantial impact on the background error rates within the amplicons. Even more, a minimum depth of 10,000× was able to accurately measure AAFs down to 0.1% with no improvement with elevated coverage. However, accurate measurement of AAFs below 0.1% required depths of 25,000× to ensure significance over the background errors. Overall, a strong correlation was found of AAFs measured across a wide range of read depths, indicating that detection of AAFs of 0.01% is possible at depths greater than above 25,000×.

The assessment of error rates and the potential for false positive allele calls was extended by performing similar sequencing on DNA samples lacking mutations. As expected, these alleles were not detectable, with only the typical background error rate being detected, which is often not the same allele as the mutation, supporting the specificity of this method.

### Precise Assessment of Broad Range of AAFs in Multiple Tissues

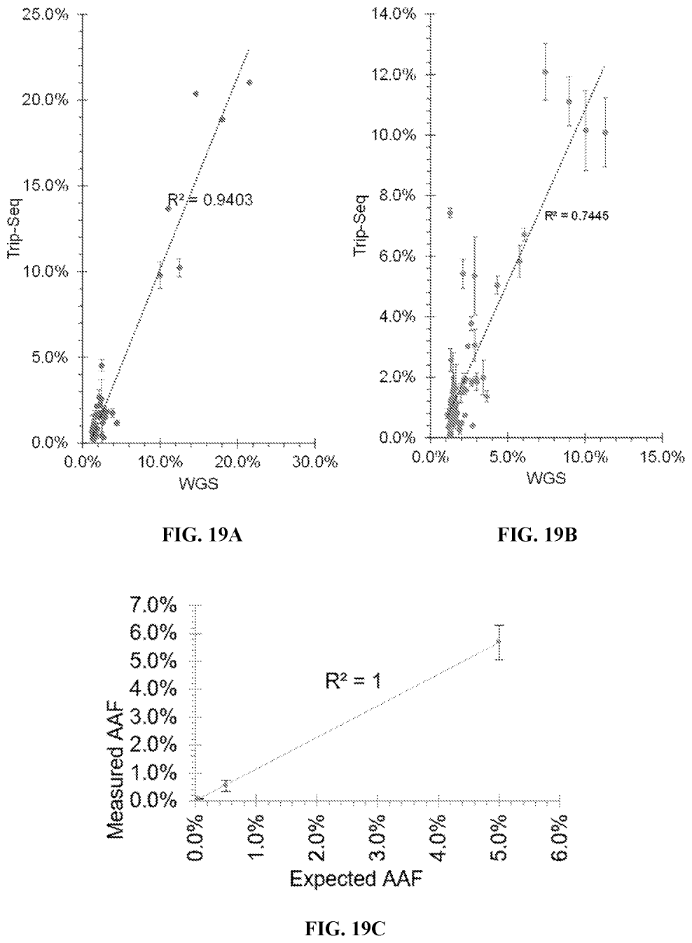

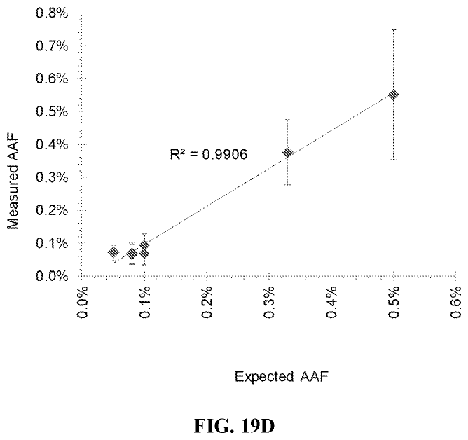

As some tissues are more difficult to work with, the ability was assessed of the method to accurately detect known mosaic alleles that were previously identified in blood and brain tissue by a range of methods including WGS, WES, and targeted Illumina sequencing. Even more, given the importance of validating indels and the elevated indels error rates on Ion Torrent data, >50 somatic indels were tested using the method of the present invention with a direct comparison of the sites between the DNA sample containing the mutation and a control sample. It was demonstrated that AAFs of SNVs (R=0.93, (FIG. 17A) and indels (R=0.89, across insertions and deletions (FIGS. 19A, 19B)) detected between the methods were highly correlated regardless of the tissue or original sequencing platform Surprisingly, very accurate assessments of indels with very little increase in error rates were obtained. However, the ability to validate extremely low AAF indels occurring within homopolymers remained challenging when using Ion Torrent. In some instances, AAFs were observed that were dissimilar to the original detection method. In these instances, the discrepancy was driven by low coverage in the original sequencing platform, resulting in an incorrect estimate of AAFs. Additionally, in some cases, a single primer provided an outlier AAF, which deviated from the other primers and original method of identification. In these cases, other primers revealed a germline mutation impacting the primer binding, resulting in allelic dropout. Such instances of allelic dropout are mitigated through the primer design process, but as often is the case, not all alleles are known, particularly in targeted sequencing and exome studies. The chances of allelic dropout highlight the importance of using multiple primers when studying mosaic and germline alleles.

### Robust Validation for Low AAF Insertions/Deletions

The known increased error rates for indel in Ion Torrent data and the inability to utilize PCR duplicate information may limit the ability to quantitate some ultra-rare alleles (<0.05% AAF) and indels. Even more, the Pollux software is known to overcorrect for indels and has difficulty distinguishing rare indels from artifacts. Despite these limitations, it was assessed how the method performs on a wide range of indels occurring at AAFs from 1% to 30% and 1 to 21 base pairs in length, including 40 insertions and 60 deletions previously identified using 200× whole genome sequencing. Even more importantly, these mutations were not identified in control DNA, where at these sites very low error rates for indels (0.010%±0.05%) were found, supporting that even the single base indels are not being introduced by PCR or the Ion Torrent. These data indicate a sensitivity to accurately quantitate AAFs of indels down to 0.05% in many instances. Despite that many of these mutations were detected using only a few reads in the WGS data, a strong correlation was found between the predicted AAFs in the WGS and the measured values by the method described in this example (FIGS. 19A, 19B; R²=0.75 deletions and R²=0.94 for insertions), indicating that this method is also sensitive to detect very low AAF indels, which are often difficult to validate.

To further improve the sensitivity for low AAFs, a modified version of the protocol was performed (FIG. 5A) in which an initial low cycle PCR was performed containing biotinylated dCTP (˜25% of a cytosines) and using unique molecular indexes (UMIs) to uniquely tag all PCR products in the first 10 cycles. After purification using either streptavidin capture or enzymatic digest (see methods), all reactions were further amplified by a common primer that maintained the UMI signature, effectively tagging all PCR duplicates from the second round of PCR. An optional step after purification comprises analyzing the sample for acceptable quality control, which, for example, can be done using a Bioanalyzer or TapeStation (FIG. 5B)

The incorporation of biotin into the PCR product did not impact the overall measured AAFs, but slightly reduced the error rate (0.0023%±0.0011% AAF), possibly due to the ability to perform better purification and the use of a common primer for the majority of the amplifications. These indicate that a 2-step UMI approach for the method is valuable in situations requiring reduced error rates for ultra-low AAFs or where PCR duplicates may be of particular concern.

### Application of Method for Novel Variant Discovery Using Illumina Sequencing

The increased sensitivity of the the presently described approach can be further applied for the detection of novel ultra-low AAFs variants with Illumina-based sequencing. Overlapping primers were developed so that all regions of the PRNP gene was covered by at least 3 independent amplicons, each containing Illumina sequencing adapters and UMIs. Using the 2-step PCR approach, sequencing libraries were prepared for a dilution series of a known mutation (5%, 0.5%, and 0.05% AAFs) and additional samples were screened for novel alleles. While any given amplicon can have some errors, as outlined above and previously documented in amplicon-based sequencing studies, it was contemplated whether the method could reduce such effects to identify high-confidence mutations. By requiring consistent AAFs across multiple unique primer sets, the AAFs of mutations were accurately measured down to at least 0.05% (FIG. 19C). Even more, when applied to a large set of tissues derived DNA samples for detections of novel mutations in a given gene, mutations down to 0.05% AAFs were accurately detected with no additional false positive occurrences (FIGS. 19C and 19D), indicating a possible option for improved accurate measurement of AAFs of novel alleles in targeted sequencing platforms.

The following materials and methods were used in carrying out this example.

### Primer Design

At least three unique sets of primers were designed for each mutation by extracting the flanking sequence around each mutation so that the mutation is located at different positions within each of the three sequences. Next, common alleles are masked, along with the targeted mutation and flanking 5bps on each site using the bedtools maskfasta tool. The masked multi-fasta file containing all sequences for targeted alleles are input into BatchPrimer webtool to design primers for each sequence. Primers are designed to an average TM of 60° C., with a minimum of 59° C. and maximum of 62° C. The amplicon length is dependent on the specific mutation and DNA sources. For example, difficult to map regions may have longer products while degraded DNA samples may require shorter amplicons. In general, to ensure that all primers are likely unique and of similar amplicon length, amplicons have a target length of 225-300 bp in length. The primer sequences are checked by BLAT and in-silico PCR to ensure both their unique amplificon in the genome and that the primer binding sites do not overlap between any set of primers. The final set of primers are then uniquely barcoded using 10 nt barcodes and if desired, an additional 10 nt UMI is added. Finally, Ion Torrent specific adapter sequences are appended to the forward and reverse primers, allowing for their direct sequencing.

### Library Preparation

For the standard, single step PCR sequencing method described above, PCR was performed using 20 cycles on a 25 μl reaction mix containing either 25 or 50 ng of input DNA sample, Phusion Hot-Start polymerase, dNTPs, HC-Buffer, and the primers. For initial testing, 30 cycles of enrichment were used to ensure only a single amplicon is produced. The high-sensitivity method modifies this process by reduction of the PCR cycling to 5 and the incorporation of 0.1 μL of 0.4 mM biotin-14-dCTP into the reaction mix. Biotinylated PCR amplicons are captured by adding 5 μl of washed Strepatvidin Myone beads resuspended in 25 μl of 2× binding and washing buffer. The mixture is incubated at room temperature with gentle mixing for 15 minutes and placed on a 96-well magnetic plate. The liquid was removed and the beads were washed one time with 1× binding and washing buffer. Then beads are then resuspended in 25 μl PCR reaction mixture containing custom primers which preserve the original UMI sequences, Phusion Hot-Start polymerase, dNTPs, and HC-Buffer. The biotin labeled product was amplified with an additional 20 cycles of enrichment before the beads were removed. Enriched products were pools at equal volumes and purified using the MagJet purification kit.

### QC and Variant Calling

Purified library pools are analyzed for enrichment efficiency and the complete removal of primers through by either the Agilent Bioanalyzer Hi-sensitivity chip or the TapeStation. The concentration was determined using PicoGreen. Pools were diluted to a final concentration of 100 pM prior to sequencing on the 430 chip for the Ion Torrent S5.

Raw unmapped bam files were obtained for each run and were processed using our custom analyses pipeline. First, all BAMs are converted to a fastq fiel using bedtools bamtofastq tool. Then, quality and adapter trimming was performed using cutadapt tool. Next, samples lacking UMIs, are demultiplexed using fastx_barcode_splitter, resulting in separate fastq files for each primer set. The barcode sequences are removed from the sequences using cutadapt. If the allele being tested in an SNV, indel correction is performed using Pollux. Finally, all samples are aligned to the reference genome using BWA-mem.

Variants are then called across the length of each amplicon though the use of samtools mPileup with the settings: q=20, Q=20. The resulting vcfs are parsed into a file containing the flanking 50 nt positions on each side of the variant and a separate file for the allele of interest. The average allele frequency across the flanking regions are then compared to the average AAF of the mutation across the 3 unique primers.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for determining alternate allele frequency, the method comprising:
a) performing two or more parallel amplification reactions on a single sample, thereby generating overlapping amplicons, wherein each amplification reaction comprises a unique pair of forward and reverse primers, wherein the forward or reverse primer comprises an index sequence, and wherein the forward and reverse primers comprise different adapter sequences;
b) sequencing the overlapping amplicons to produce sequence reads;
c) segregating the sequencing reads into bins by index sequence; and
d) detecting the presence or absence of one or more genetic variants within sequencing reads within a bin, wherein the frequency of detection of the variant determines the alternate allele frequency.

2. A method for determining alternate allele frequency, the method comprising:
a) performing three amplification reactions on a single sample, thereby generating three overlapping amplicons, wherein each amplification reaction comprises a unique pair of forward and reverse primers, wherein each primer comprises a nucleic acid sequence complementary to a portion of a target nucleic acid sequence, wherein the forward or reverse primer comprises an index sequence, and wherein the forward and reverse primers comprise different adapter sequences at or near the 5′ terminus of the primer and upstream of the sequence complementary to the target, and wherein at least one adapter sequence is complementary to a nucleic acid sequence used in sequencing;
b) sequencing the overlapping amplicons to produce sequence reads;
c) segregating the sequencing reads into bins by index sequence; and
d) detecting the presence or absence of one or more genetic variants within sequencing reads within a bin, wherein the frequency of detection of the variant determines the alternate allele frequency.

3. A method for determining alternate allele frequency, the method comprising:
a) performing three amplification reactions on a single sample, thereby generating three overlapping amplicons, wherein each amplification reaction comprises a unique pair of forward and reverse primers, wherein the forward or reverse primer comprises an index sequence and/or a unique molecular identifier (UMI); and each primer comprises
i. a nucleotide sequence complementary to a portion of a target nucleic acid sequence;
ii. an adapter at or near its 5′ terminus, wherein the adapter is upstream of the sequence complementary to the target and wherein the forward and reverse primers comprise different adapter sequences, wherein at least one adapter sequence is complementary to a nucleic acid sequence used in sequencing;
b) sequencing the overlapping amplicons to produce sequence reads;
c) segregating the sequencing reads into bins by index sequence;
d) detecting the UMI and removing duplicate reads from the bin, wherein the detecting can be simultaneous with step c or subsequent to step c; and
e) detecting the presence or absence of one or more genetic variants within sequencing reads within a bin, wherein the frequency of detection of the variant determines the alternate allele frequency.

4. The method of claim 1 further comprising pooling the amplicons prior to sequencing.

5. The method of claim 1, wherein sequencing the amplicons comprises contacting the amplicons with a nucleic acid complementary to the adapter sequence.

6. The method of claim 1, wherein the amplicons comprise a nucleotide having a label, optionally wherein the label is biotin.

7. (canceled)

8. The method of claim 6 further comprising contacting the label with a capture agent that specifically binds the label.

9. The method of claim 1 further comprising enzymatically digesting the primers.

10. The method of claim 1 further comprising amplifying the amplicons, thereby generating enriched populations of amplicons.

11. The method of claim 1, wherein the genetic variation to be detected is known or unknown.

12. The method of claim 1, wherein the genetic variant has an alternate allele fraction of at least 0.1%.

13. The method of claim 1, wherein the genetic variant has an alternate allele fraction of at least 0.025%.

14. The method of claim 1, wherein the genetic variant is a mosaic variant.

15. The method of claim 1, wherein detection of the genetic variant identifies the presence of a disease or a predisposition to a disease in a subject from whom the sample was derived.

16. The method of claim 15, wherein the disease is cancer.

17. The method claim 1, wherein the sample comprises circulating tumor cells or cell free DNA.

18. The method of claim 1, wherein the genetic variant originated from a somatic event or a germline event.

19. The method of claim 15, wherein the alternate allele frequency is compared to the allele frequency of a reference sample to determine if the subject's disease is progressing, regressing, or in remission.

20. The method of claim 1 further comprising averaging the alternate allele frequencies determined for each bin.

21. The method of claim 20 further comprising determining the error rate of the nucleic acid sequences flanking the alternate allele.


SEQUENCE LISTING (339 sequences)

SEQ ID NO: 1 - DNA, 18 bp, Artificial Sequence
  Description of Artificial Sequence Synthetic primer
cagggcctcaccttggtc

SEQ ID NO: 2 - DNA, 22 bp, Artificial Sequence
  Description of Artificial Sequence Synthetic primer
ctgtgacatagcactcctccag

SEQ ID NO: 3 - DNA, 18 bp, Artificial Sequence
  Description of Artificial Sequence Synthetic primer
aggctggctggttgacct

SEQ ID NO: 4 - DNA, 26 bp, Artificial Sequence
  Description of Artificial Sequence Synthetic primer
aatcacactccataggtatcatttca

SEQ ID NO: 5 - DNA, 25 bp, Artificial Sequence
  Description of Artificial Sequence Synthetic primer
ttcattcatttgtttaagatcagca

SEQ ID NO: 6 - DNA, 28 bp, Artificial Sequence
  Description of Artificial Sequence Synthetic primer
ttaacggacgcgccagatgggtaagtgc

SEQ ID NO: 7 - DNA, 29 bp, Artificial Sequence
  Description of Artificial Sequence Synthetic primer
tccggcttactgcaaatcagtggctctcc

SEQ ID NO: 8 - DNA, 28 bp, Artificial Sequence
  Description of Artificial Sequence Synthetic primer
tctcattcagctcccttcctgccacctg

SEQ ID NO: 9 - DNA, 35 bp, Artificial Sequence
  Description of Artificial Sequence Synthetic primer
gcggtcatacacatgtgatacttttgggaatgaag

SEQ ID NO: 10 - DNA, 32 bp, Artificial Sequence
  Description of Artificial Sequence Synthetic primer
taggacgttccttctgaacaccaaattggaaa

SEQ ID NO: 11 - DNA, 15 bp, Artificial Sequence
  Description of Artificial Sequence Synthetic oligonucleotide
ttaacggacgcgcca

SEQ ID NO: 12 - DNA, 15 bp, Artificial Sequence
  Description of Artificial Sequence Synthetic oligonucleotide
tccggcttactgcaa

SEQ ID NO: 13 - DNA, 15 bp, Artificial Sequence
  Description of Artificial Sequence Synthetic oligonucleotide
tctcattcagctccc

SEQ ID NO: 14 - DNA, 15 bp, Artificial Sequence
  Description of Artificial Sequence Synthetic oligonucleotide
gcggtcatacacatg

SEQ ID NO: 15 - DNA, 15 bp, Artificial Sequence
  Description of Artificial Sequence Synthetic oligonucleotide
taggacgttccttct

SEQ ID NO: 16 - DNA, 15 bp, Artificial Sequence
  Description of Artificial Sequence Synthetic oligonucleotide
caaggtgaggccctg

SEQ ID NO: 17 - DNA, 15 bp, Artificial Sequence
  Description of Artificial Sequence Synthetic oligonucleotide
agtgctatgtcacag

SEQ ID NO: 18 - DNA, 15 bp, Artificial Sequence
  Description of Artificial Sequence Synthetic oligonucleotide
tcaaccagccagcct

SEQ ID NO: 19 - DNA, 15 bp, Artificial Sequence
  Description of Artificial Sequence Synthetic oligonucleotide
ctatggagtgtgatt

SEQ ID NO: 20 - DNA, 15 bp, Artificial Sequence
  Description of Artificial Sequence Synthetic oligonucleotide
aaacaaatgaatgaa

SEQ ID NO: 21 - DNA, 41 bp, Artificial Sequence
  Description of Artificial Sequence Synthetic primer
cctctctatgggcagtcggtgatcagggcctcaccttggtc

SEQ ID NO: 22 - DNA, 45 bp, Artificial Sequence
  Description of Artificial Sequence Synthetic primer
cctctctatgggcagtcggtgatctgtgacatagcactcctccag

SEQ ID NO: 23 - DNA, 41 bp, Artificial Sequence
  Description of Artificial Sequence Synthetic primer
cctctctatgggcagtcggtgataggctggctggttgacct

SEQ ID NO: 24 - DNA, 49 bp, Artificial Sequence
  Description of Artificial Sequence Synthetic primer
cctctctatgggcagtcggtgataatcacactccataggtatcatttca

SEQ ID NO: 25 - DNA, 48 bp, Artificial Sequence
  Description of Artificial Sequence Synthetic primer
cctctctatgggcagtcggtgatttcattcatttgtttaagatcagca

SEQ ID NO: 26 - DNA, 46 bp, Artificial Sequence
  Description of Artificial Sequence Synthetic primer
cctctctatgggcagtcggtgattcaatgtgtgttttaggcaactc

SEQ ID NO: 27 - DNA, 41 bp, Artificial Sequence
  Description of Artificial Sequence Synthetic primer
cctctctatgggcagtcggtgatagggggacatgcaagaca

SEQ ID NO: 28 - DNA, 41 bp, Artificial Sequence
  Description of Artificial Sequence Synthetic primer
cctctctatgggcagtcggtgatgcgagctcttccgaaggt

SEQ ID NO: 29 - DNA, 41 bp, Artificial Sequence
  Description of Artificial Sequence Synthetic primer
cctctctatgggcagtcggtgatgttgaccctgtgggcaga

SEQ ID NO: 30 - DNA, 43 bp, Artificial Sequence
  Description of Artificial Sequence Synthetic primer
cctctctatgggcagtcggtgattcctctgggtagtgcagctt

SEQ ID NO: 31 - DNA, 43 bp, Artificial Sequence
  Description of Artificial Sequence Synthetic primer
cctctctatgggcagtcggtgatcagagccgaattcagaaagc

SEQ ID NO: 32 - DNA, 41 bp, Artificial Sequence
  Description of Artificial Sequence Synthetic primer
cctctctatgggcagtcggtgatggggtggtgtccaggagt

SEQ ID NO: 33 - DNA, 41 bp, Artificial Sequence
  Description of Artificial Sequence Synthetic primer
cctctctatgggcagtcggtgatcattttgaggcgcgagaa

SEQ ID NO: 34 - DNA, 41 bp, Artificial Sequence
  Description of Artificial Sequence Synthetic primer
cctctctatgggcagtcggtgatgaggcagggagcagaggt

SEQ ID NO: 35 - DNA, 42 bp, Artificial Sequence
  Description of Artificial Sequence Synthetic primer
cctctctatgggcagtcggtgatcctttaaatgcgggaggag

SEQ ID NO: 36 - DNA, 44 bp, Artificial Sequence
  Description of Artificial Sequence Synthetic primer
cctctctatgggcagtcggtgatgagcaggaagggcaggtataa

SEQ ID NO: 37 - DNA, 42 bp, Artificial Sequence
  Description of Artificial Sequence Synthetic primer
cctctctatgggcagtcggtgatctggcacaggctgactcat

SEQ ID NO: 38 - DNA, 41 bp, Artificial Sequence
  Description of Artificial Sequence Synthetic primer
cctctctatgggcagtcggtgatggatgcctccagcagtga

SEQ ID NO: 39 - DNA, 45 bp, Artificial Sequence
  Description of Artificial Sequence Synthetic primer
cctctctatgggcagtcggtgatagcctcataagggatgtactcg

SEQ ID NO: 40 - DNA, 43 bp, Artificial Sequence
  Description of Artificial Sequence Synthetic primer
cctctctatgggcagtcggtgatccttgaaaggactgcctgag

SEQ ID NO: 41 - DNA, 43 bp, Artificial Sequence
  Description of Artificial Sequence Synthetic primer
cctctctatgggcagtcggtgatctcctcacctggcacttgat

SEQ ID NO: 42 - DNA, 43 bp, Artificial Sequence
  Description of Artificial Sequence Synthetic primer
cctctctatgggcagtcggtgatagagcccacacaggagagag

SEQ ID NO: 43 - DNA, 42 bp, Artificial Sequence
  Description of Artificial Sequence Synthetic primer
cctctctatgggcagtcggtgatatcagcgagtccacactgg

SEQ ID NO: 44 - DNA, 47 bp, Artificial Sequence
  Description of Artificial Sequence Synthetic primer
cctctctatgggcagtcggtgatttgaatcataagagactccattgc

SEQ ID NO: 45 - DNA, 58 bp, Artificial Sequence
  Description of Artificial Sequence Synthetic primer
ccatctcatccctgcgtgtctccgactcagttaacggacgcgccagatgggtaagtgc

SEQ ID NO: 46 - DNA, 59 bp, Artificial Sequence
  Description of Artificial Sequence Synthetic primer
ccatctcatccctgcgtgtctccgactcagtccggcttactgcaaatcagtggctctcc

SEQ ID NO: 47 - DNA, 58 bp, Artificial Sequence
  Description of Artificial Sequence Synthetic primer
ccatctcatccctgcgtgtctccgactcagtctcattcagctcccttcctgccacctg

SEQ ID NO: 48 - DNA, 65 bp, Artificial Sequence
  Description of Artificial Sequence Synthetic primer
ccatctcatccctgcgtgtctccgactcaggcggtcatacacatgtgatacttttgggaatgaag

SEQ ID NO: 49 - DNA, 62 bp, Artificial Sequence
  Description of Artificial Sequence Synthetic primer
ccatctcatccctgcgtgtctccgactcagtaggacgttccttctgaacaccaaattggaaa

SEQ ID NO: 50 - DNA, 61 bp, Artificial Sequence
  Description of Artificial Sequence Synthetic primer
ccatctcatccctgcgtgtctccgactcagacgacgcaactgttaagagcccagaggttca

SEQ ID NO: 51 - DNA, 58 bp, Artificial Sequence
  Description of Artificial Sequence Synthetic primer
ccatctcatccctgcgtgtctccgactcagcttctcggacggggcccctactctttga

SEQ ID NO: 52 - DNA, 58 bp, Artificial Sequence
  Description of Artificial Sequence Synthetic primer
ccatctcatccctgcgtgtctccgactcagcattgccgttctcgcagcccctacactg

SEQ ID NO: 53 - DNA, 58 bp, Artificial Sequence
  Description of Artificial Sequence Synthetic primer
ccatctcatccctgcgtgtctccgactcagcgagccagaatgactgccctctgctgtg

SEQ ID NO: 54 - DNA, 60 bp, Artificial Sequence
  Description of Artificial Sequence Synthetic primer
ccatctcatccctgcgtgtctccgactcagtgaggacggcagtgacgatgagcaggaggt

SEQ ID NO: 55 - DNA, 58 bp, Artificial Sequence
  Description of Artificial Sequence Synthetic primer
ccatctcatccctgcgtgtctccgactcaggcctgcgcaggccaattcccattgacca

SEQ ID NO: 56 - DNA, 60 bp, Artificial Sequence
  Description of Artificial Sequence Synthetic primer
ccatctcatccctgcgtgtctccgactcaggttgacgtctccaagcttcctgaaccagac

SEQ ID NO: 57 - DNA, 58 bp, Artificial Sequence
  Description of Artificial Sequence Synthetic primer
ccatctcatccctgcgtgtctccgactcaggagatcgattctagtgggggcattccaa

SEQ ID NO: 58 - DNA, 58 bp, Artificial Sequence
  Description of Artificial Sequence Synthetic primer
ccatctcatccctgcgtgtctccgactcagagttcgagccctctagggcgcgtttcct

SEQ ID NO: 59 - DNA, 59 bp, Artificial Sequence
  Description of Artificial Sequence Synthetic primer
ccatctcatccctgcgtgtctccgactcagctcaggctcatcagcctttcctcgctcta

SEQ ID NO: 60 - DNA, 60 bp, Artificial Sequence
  Description of Artificial Sequence Synthetic primer
ccatctcatccctgcgtgtctccgactcagggcaatataatccacataactcgcttgcag

SEQ ID NO: 61 - DNA, 61 bp, Artificial Sequence
  Description of Artificial Sequence Synthetic primer
ccatctcatccctgcgtgtctccgactcagggtactcatggaactgtagcccagacactgc

SEQ ID NO: 62 - DNA, 63 bp, Artificial Sequence
  Description of Artificial Sequence Synthetic primer
ccatctcatccctgcgtgtctccgactcagtctggttcaaacaaagctggaaactcttcccta

SEQ ID NO: 63 - DNA, 60 bp, Artificial Sequence
  Description of Artificial Sequence Synthetic primer
ccatctcatccctgcgtgtctccgactcaggtcctataagccaacaagcccaacaagttc

SEQ ID NO: 64 - DNA, 60 bp, Artificial Sequence
  Description of Artificial Sequence Synthetic primer
ccatctcatccctgcgtgtctccgactcaggtcagcctccgaatgaccggctgtctgttt

SEQ ID NO: 65 - DNA, 59 bp, Artificial Sequence
  Description of Artificial Sequence Synthetic primer
ccatctcatccctgcgtgtctccgactcagttcaagctcgaaagtggcaccaccaacaa

SEQ ID NO: 66 - DNA, 60 bp, Artificial Sequence
  Description of Artificial Sequence Synthetic primer
ccatctcatccctgcgtgtctccgactcaggtaccagcgccttgtagcgcttcccacagt

SEQ ID NO: 67 - DNA, 61 bp, Artificial Sequence
  Description of Artificial Sequence Synthetic primer
ccatctcatccctgcgtgtctccgactcagtcctattcggagcttctttccacaatcctca

SEQ ID NO: 68 - DNA, 67 bp, Artificial Sequence
  Description of Artificial Sequence Synthetic primer
ccatctcatccctgcgtgtctccgactcaggccagcgattctgtaccccataaatatgtacaacact

SEQ ID NO: 69 - DNA, 58 bp, Artificial Sequence
  Description of Artificial Sequence Synthetic primer
ccatctcatccctgcgtgtctccgactcagacctagactgcgccagatgggtaagtgc

SEQ ID NO: 70 - DNA, 59 bp, Artificial Sequence
  Description of Artificial Sequence Synthetic primer
ccatctcatccctgcgtgtctccgactcagactggttcgctgcaaatcagtggctctcc

SEQ ID NO: 71 - DNA, 58 bp, Artificial Sequence
  Description of Artificial Sequence Synthetic primer
ccatctcatccctgcgtgtctccgactcagccatattaggctcccttcctgccacctg

SEQ ID NO: 72 - DNA, 65 bp, Artificial Sequence
  Description of Artificial Sequence Synthetic primer
ccatctcatccctgcgtgtctccgactcaggctcgtcagcacatgtgatacttttgggaatgaag

SEQ ID NO: 73 - DNA, 62 bp, Artificial Sequence
  Description of Artificial Sequence Synthetic primer
ccatctcatccctgcgtgtctccgactcagcgtaatgacgcttctgaacaccaaattggaaa

SEQ ID NO: 74 - DNA, 61 bp, Artificial Sequence
  Description of Artificial Sequence Synthetic primer
ccatctcatccctgcgtgtctccgactcagccggcgctgatgttaagagcccagaggttca

SEQ ID NO: 75 - DNA, 58 bp, Artificial Sequence
  Description of Artificial Sequence Synthetic primer
ccatctcatccctgcgtgtctccgactcagcgcgaagataggggcccctactctttga

SEQ ID NO: 76 - DNA, 58 bp, Artificial Sequence
  Description of Artificial Sequence Synthetic primer
ccatctcatccctgcgtgtctccgactcaggaaccgcagactcgcagcccctacactg

SEQ ID NO: 77 - DNA, 58 bp, Artificial Sequence
  Description of Artificial Sequence Synthetic primer
ccatctcatccctgcgtgtctccgactcagttggcagagatgactgccctctgctgtg

SEQ ID NO: 78 - DNA, 60 bp, Artificial Sequence
  Description of Artificial Sequence Synthetic primer
ccatctcatccctgcgtgtctccgactcaggcatctctgcagtgacgatgagcaggaggt

SEQ ID NO: 79 - DNA, 58 bp, Artificial Sequence
  Description of Artificial Sequence Synthetic primer
ccatctcatccctgcgtgtctccgactcagttggaccgcagccaattcccattgacca

SEQ ID NO: 80 - DNA, 60 bp, Artificial Sequence
  Description of Artificial Sequence Synthetic primer
ccatctcatccctgcgtgtctccgactcaggcagaacgtcccaagcttcctgaaccagac

SEQ ID NO: 81 - DNA, 58 bp, Artificial Sequence
  Description of Artificial Sequence Synthetic primer
ccatctcatccctgcgtgtctccgactcagaacttcgagcctagtgggggcattccaa

SEQ ID NO: 82 - DNA, 58 bp, Artificial Sequence
  Description of Artificial Sequence Synthetic primer
ccatctcatccctgcgtgtctccgactcaggctcctagagctctagggcgcgtttcct

SEQ ID NO: 83 - DNA, 59 bp, Artificial Sequence
  Description of Artificial Sequence Synthetic primer
ccatctcatccctgcgtgtctccgactcagtatctagctttcagcctttcctcgctcta

SEQ ID NO: 84 - DNA, 60 bp, Artificial Sequence
  Description of Artificial Sequence Synthetic primer
ccatctcatccctgcgtgtctccgactcaggagtattggctccacataactcgcttgcag

SEQ ID NO: 85 - DNA, 61 bp, Artificial Sequence
  Description of Artificial Sequence Synthetic primer
ccatctcatccctgcgtgtctccgactcagcctgagctcagaactgtagcccagacactgc

SEQ ID NO: 86 - DNA, 63 bp, Artificial Sequence
  Description of Artificial Sequence Synthetic primer
ccatctcatccctgcgtgtctccgactcagcaggcgagtaacaaagctggaaactcttcccta

SEQ ID NO: 87 - DNA, 60 bp, Artificial Sequence
  Description of Artificial Sequence Synthetic primer
ccatctcatccctgcgtgtctccgactcaggcaggcagagccaacaagcccaacaagttc

SEQ ID NO: 88 - DNA, 60 bp, Artificial Sequence
  Description of Artificial Sequence Synthetic primer
ccatctcatccctgcgtgtctccgactcaggcgtcgatacgaatgaccggctgtctgttt

SEQ ID NO: 89 - DNA, 59 bp, Artificial Sequence
  Description of Artificial Sequence Synthetic primer
ccatctcatccctgcgtgtctccgactcagcgatgattataaagtggcaccaccaacaa

SEQ ID NO: 90 - DNA, 60 bp, Artificial Sequence
  Description of Artificial Sequence Synthetic primer
ccatctcatccctgcgtgtctccgactcaggacggctggccttgtagcgcttcccacagt

SEQ ID NO: 91 - DNA, 61 bp, Artificial Sequence
  Description of Artificial Sequence Synthetic primer
ccatctcatccctgcgtgtctccgactcagggagcctgagagcttctttccacaatcctca

SEQ ID NO: 92 - DNA, 67 bp, Artificial Sequence
  Description of Artificial Sequence Synthetic primer
ccatctcatccctgcgtgtctccgactcagcctgactgctctgtaccccataaatatgtacaacact

SEQ ID NO: 93 - DNA, 58 bp, Artificial Sequence
  Description of Artificial Sequence Synthetic primer
ccatctcatccctgcgtgtctccgactcagacggctgacgcgccagatgggtaagtgc

SEQ ID NO: 94 - DNA, 59 bp, Artificial Sequence
  Description of Artificial Sequence Synthetic primer
ccatctcatccctgcgtgtctccgactcagtaaccatagctgcaaatcagtggctctcc

SEQ ID NO: 95 - DNA, 58 bp, Artificial Sequence
  Description of Artificial Sequence Synthetic primer
ccatctcatccctgcgtgtctccgactcagtcttgccttcctcccttcctgccacctg

SEQ ID NO: 96 - DNA, 65 bp, Artificial Sequence
  Description of Artificial Sequence Synthetic primer
ccatctcatccctgcgtgtctccgactcagttcttagattacatgtgatacttttgggaatgaag

SEQ ID NO: 97 - DNA, 62 bp, Artificial Sequence
  Description of Artificial Sequence Synthetic primer
ccatctcatccctgcgtgtctccgactcagtcatctcattcttctgaacaccaaattggaaa

SEQ ID NO: 98 - DNA, 61 bp, Artificial Sequence
  Description of Artificial Sequence Synthetic primer
ccatctcatccctgcgtgtctccgactcagtctccgctcgtgttaagagcccagaggttca

SEQ ID NO: 99 - DNA, 58 bp, Artificial Sequence
  Description of Artificial Sequence Synthetic primer
ccatctcatccctgcgtgtctccgactcagtgccatatgcggggcccctactctttga

SEQ ID NO: 100 - DNA, 58 bp, Artificial Sequence
  Description of Artificial Sequence Synthetic primer
ccatctcatccctgcgtgtctccgactcagtaaggcctctctcgcagcccctacactg

SEQ ID NO: 101 - DNA, 58 bp, Artificial Sequence
  Description of Artificial Sequence Synthetic primer
ccatctcatccctgcgtgtctccgactcaggagtaggccgtgactgccctctgctgtg

SEQ ID NO: 102 - DNA, 60 bp, Artificial Sequence
  Description of Artificial Sequence Synthetic primer
ccatctcatccctgcgtgtctccgactcaggcaataagctagtgacgatgagcaggaggt

SEQ ID NO: 103 - DNA, 58 bp, Artificial Sequence
  Description of Artificial Sequence Synthetic primer
ccatctcatccctgcgtgtctccgactcagggcgttgcaagccaattcccattgacca

SEQ ID NO: 104 - DNA, 60 bp, Artificial Sequence
  Description of Artificial Sequence Synthetic primer
ccatctcatccctgcgtgtctccgactcagccaagaagcgccaagcttcctgaaccagac

SEQ ID NO: 105 - DNA, 58 bp, Artificial Sequence
  Description of Artificial Sequence Synthetic primer
ccatctcatccctgcgtgtctccgactcagggttacctcgctagtgggggcattccaa

SEQ ID NO: 106 - DNA, 58 bp, Artificial Sequence
  Description of Artificial Sequence Synthetic primer
ccatctcatccctgcgtgtctccgactcagctccgccttactctagggcgcgtttcct

SEQ ID NO: 107 - DNA, 59 bp, Artificial Sequence
  Description of Artificial Sequence Synthetic primer
ccatctcatccctgcgtgtctccgactcagctccagagattcagcctttcctcgctcta

SEQ ID NO: 108 - DNA, 60 bp, Artificial Sequence
  Description of Artificial Sequence Synthetic primer
ccatctcatccctgcgtgtctccgactcaggtcgaggtagtccacataactcgcttgcag

SEQ ID NO: 109 - DNA, 61 bp, Artificial Sequence
  Description of Artificial Sequence Synthetic primer
ccatctcatccctgcgtgtctccgactcagtatggacctggaactgtagcccagacactgc

SEQ ID NO: 110 - DNA, 63 bp, Artificial Sequence
  Description of Artificial Sequence Synthetic primer
ccatctcatccctgcgtgtctccgactcagtacctgctagacaaagctggaaactcttcccta

SEQ ID NO: 111 - DNA, 60 bp, Artificial Sequence
  Description of Artificial Sequence Synthetic primer
ccatctcatccctgcgtgtctccgactcagccgcgaccgaccaacaagcccaacaagttc

SEQ ID NO: 112 - DNA, 60 bp, Artificial Sequence
  Description of Artificial Sequence Synthetic primer
ccatctcatccctgcgtgtctccgactcaggttgaacgttgaatgaccggctgtctgttt

SEQ ID NO: 113 - DNA, 59 bp, Artificial Sequence
  Description of Artificial Sequence Synthetic primer
ccatctcatccctgcgtgtctccgactcagtgccaacgcaaaagtggcaccaccaacaa

SEQ ID NO: 114 - DNA, 60 bp, Artificial Sequence
  Description of Artificial Sequence Synthetic primer
ccatctcatccctgcgtgtctccgactcagggattgacctcttgtagcgcttcccacagt

SEQ ID NO: 115 - DNA, 61 bp, Artificial Sequence
  Description of Artificial Sequence Synthetic primer
ccatctcatccctgcgtgtctccgactcagggacggattcagcttctttccacaatcctca

SEQ ID NO: 116 - DNA, 67 bp, Artificial Sequence
  Description of Artificial Sequence Synthetic primer
ccatctcatccctgcgtgtctccgactcagtcctccgtcgctgtaccccataaatatgtacaacact

SEQ ID NO: 117 - DNA, 58 bp, Artificial Sequence
  Description of Artificial Sequence Synthetic primer
ccatctcatccctgcgtgtctccgactcagagttcatggtcgccagatgggtaagtgc

SEQ ID NO: 118 - DNA, 59 bp, Artificial Sequence
  Description of Artificial Sequence Synthetic primer
ccatctcatccctgcgtgtctccgactcagtatccattcctgcaaatcagtggctctcc

SEQ ID NO: 119 - DNA, 58 bp, Artificial Sequence
  Description of Artificial Sequence Synthetic primer
ccatctcatccctgcgtgtctccgactcagggagagcgcgctcccttcctgccacctg

SEQ ID NO: 120 - DNA, 65 bp, Artificial Sequence
  Description of Artificial Sequence Synthetic primer
ccatctcatccctgcgtgtctccgactcagcggaccttggacatgtgatacttttgggaatgaag

SEQ ID NO: 121 - DNA, 62 bp, Artificial Sequence
  Description of Artificial Sequence Synthetic primer
ccatctcatccctgcgtgtctccgactcagggcaatctcccttctgaacaccaaattggaaa

SEQ ID NO: 122 - DNA, 61 bp, Artificial Sequence
  Description of Artificial Sequence Synthetic primer
ccatctcatccctgcgtgtctccgactcagaggattgatttgttaagagcccagaggttca

SEQ ID NO: 123 - DNA, 58 bp, Artificial Sequence
  Description of Artificial Sequence Synthetic primer
ccatctcatccctgcgtgtctccgactcaggccgttgcctggggcccctactctttga

SEQ ID NO: 124 - DNA, 58 bp, Artificial Sequence
  Description of Artificial Sequence Synthetic primer
ccatctcatccctgcgtgtctccgactcagaagtacgtcgctcgcagcccctacactg

SEQ ID NO: 125 - DNA, 58 bp, Artificial Sequence
  Description of Artificial Sequence Synthetic primer
ccatctcatccctgcgtgtctccgactcagtggcttaaggtgactgccctctgctgtg

SEQ ID NO: 126 - DNA, 60 bp, Artificial Sequence
  Description of Artificial Sequence Synthetic primer
ccatctcatccctgcgtgtctccgactcagctcttccagaagtgacgatgagcaggaggt

SEQ ID NO: 127 - DNA, 58 bp, Artificial Sequence
  Description of Artificial Sequence Synthetic primer
ccatctcatccctgcgtgtctccgactcagcgttcttcaagccaattcccattgacca

SEQ ID NO: 128 - DNA, 60 bp, Artificial Sequence
  Description of Artificial Sequence Synthetic primer
ccatctcatccctgcgtgtctccgactcagcaacggctgcccaagcttcctgaaccagac

SEQ ID NO: 129 - DNA, 58 bp, Artificial Sequence
  Description of Artificial Sequence Synthetic primer
ccatctcatccctgcgtgtctccgactcaggcaagtaaccctagtgggggcattccaa

SEQ ID NO: 130 - DNA, 58 bp, Artificial Sequence
  Description of Artificial Sequence Synthetic primer
ccatctcatccctgcgtgtctccgactcaggttcatagtcctctagggcgcgtttcct

SEQ ID NO: 131 - DNA, 59 bp, Artificial Sequence
  Description of Artificial Sequence Synthetic primer
ccatctcatccctgcgtgtctccgactcagacggcgagcctcagcctttcctcgctcta

SEQ ID NO: 132 - DNA, 60 bp, Artificial Sequence
  Description of Artificial Sequence Synthetic primer
ccatctcatccctgcgtgtctccgactcaggtatggtcggtccacataactcgcttgcag

SEQ ID NO: 133 - DNA, 61 bp, Artificial Sequence
  Description of Artificial Sequence Synthetic primer
ccatctcatccctgcgtgtctccgactcagtcggttatccgaactgtagcccagacactgc

SEQ ID NO: 134 - DNA, 63 bp, Artificial Sequence
  Description of Artificial Sequence Synthetic primer
ccatctcatccctgcgtgtctccgactcaggcggtcgataacaaagctggaaactcttcccta

SEQ ID NO: 135 - DNA, 60 bp, Artificial Sequence
  Description of Artificial Sequence Synthetic primer
ccatctcatccctgcgtgtctccgactcagtcctcagtatccaacaagcccaacaagttc

SEQ ID NO: 136 - DNA, 60 bp, Artificial Sequence
  Description of Artificial Sequence Synthetic primer
ccatctcatccctgcgtgtctccgactcagaccgttcctggaatgaccggctgtctgttt

SEQ ID NO: 137 - DNA, 59 bp, Artificial Sequence
  Description of Artificial Sequence Synthetic primer
ccatctcatccctgcgtgtctccgactcaggcctgctcttaaagtggcaccaccaacaa

SEQ ID NO: 138 - DNA, 60 bp, Artificial Sequence
  Description of Artificial Sequence Synthetic primer
ccatctcatccctgcgtgtctccgactcagagcgtaaccacttgtagcgcttcccacagt

SEQ ID NO: 139 - DNA, 61 bp, Artificial Sequence
  Description of Artificial Sequence Synthetic primer
ccatctcatccctgcgtgtctccgactcagttgcctgatgagcttctttccacaatcctca

SEQ ID NO: 140 - DNA, 67 bp, Artificial Sequence
  Description of Artificial Sequence Synthetic primer
ccatctcatccctgcgtgtctccgactcagttattgatctctgtaccccataaatatgtacaacact

SEQ ID NO: 141 - DNA, 58 bp, Artificial Sequence
  Description of Artificial Sequence Synthetic primer
ccatctcatccctgcgtgtctccgactcagtacgctcggacgccagatgggtaagtgc

SEQ ID NO: 142 - DNA, 59 bp, Artificial Sequence
  Description of Artificial Sequence Synthetic primer
ccatctcatccctgcgtgtctccgactcagcaatccaaggtgcaaatcagtggctctcc

SEQ ID NO: 143 - DNA, 58 bp, Artificial Sequence
  Description of Artificial Sequence Synthetic primer
ccatctcatccctgcgtgtctccgactcagtcgtagctatctcccttcctgccacctg

SEQ ID NO: 144 - DNA, 65 bp, Artificial Sequence
  Description of Artificial Sequence Synthetic primer
ccatctcatccctgcgtgtctccgactcagcgctcatcgcacatgtgatacttttgggaatgaag

SEQ ID NO: 145 - DNA, 62 bp, Artificial Sequence
  Description of Artificial Sequence Synthetic primer
ccatctcatccctgcgtgtctccgactcagtccgttcattcttctgaacaccaaattggaaa

SEQ ID NO: 146 - DNA, 61 bp, Artificial Sequence
  Description of Artificial Sequence Synthetic primer
ccatctcatccctgcgtgtctccgactcagcggccaggcttgttaagagcccagaggttca

SEQ ID NO: 147 - DNA, 58 bp, Artificial Sequence
  Description of Artificial Sequence Synthetic primer
ccatctcatccctgcgtgtctccgactcagcaacctatctggggcccctactctttga

SEQ ID NO: 148 - DNA, 58 bp, Artificial Sequence
  Description of Artificial Sequence Synthetic primer
ccatctcatccctgcgtgtctccgactcagcgtaatctcactcgcagcccctacactg

SEQ ID NO: 149 - DNA, 58 bp, Artificial Sequence
  Description of Artificial Sequence Synthetic primer
ccatctcatccctgcgtgtctccgactcagatatcgcgactgactgccctctgctgtg

SEQ ID NO: 150 - DNA, 60 bp, Artificial Sequence
  Description of Artificial Sequence Synthetic primer
ccatctcatccctgcgtgtctccgactcagtcaatatctgagtgacgatgagcaggaggt

SEQ ID NO: 151 - DNA, 58 bp, Artificial Sequence
  Description of Artificial Sequence Synthetic primer
ccatctcatccctgcgtgtctccgactcagatagagtatagccaattcccattgacca

SEQ ID NO: 152 - DNA, 60 bp, Artificial Sequence
  Description of Artificial Sequence Synthetic primer
ccatctcatccctgcgtgtctccgactcaggcaactagttccaagcttcctgaaccagac

SEQ ID NO: 153 - DNA, 58 bp, Artificial Sequence
  Description of Artificial Sequence Synthetic primer
ccatctcatccctgcgtgtctccgactcagatctcgaatcctagtgggggcattccaa

SEQ ID NO: 154 - DNA, 58 bp, Artificial Sequence
  Description of Artificial Sequence Synthetic primer
ccatctcatccctgcgtgtctccgactcagccaggagcgactctagggcgcgtttcct

SEQ ID NO: 155 - DNA, 59 bp, Artificial Sequence
  Description of Artificial Sequence Synthetic primer
ccatctcatccctgcgtgtctccgactcagatctccatcgtcagcctttcctcgctcta

SEQ ID NO: 156 - DNA, 60 bp, Artificial Sequence
  Description of Artificial Sequence Synthetic primer
ccatctcatccctgcgtgtctccgactcagttgacgagcttccacataactcgcttgcag

SEQ ID NO: 157 - DNA, 61 bp, Artificial Sequence
  Description of Artificial Sequence Synthetic primer
ccatctcatccctgcgtgtctccgactcagtactattaccgaactgtagcccagacactgc

SEQ ID NO: 158 - DNA, 63 bp, Artificial Sequence
  Description of Artificial Sequence Synthetic primer
ccatctcatccctgcgtgtctccgactcagcgtcctggacacaaagctggaaactcttcccta

SEQ ID NO: 159 - DNA, 60 bp, Artificial Sequence
  Description of Artificial Sequence Synthetic primer
ccatctcatccctgcgtgtctccgactcagctcggcgcttccaacaagcccaacaagttc

SEQ ID NO: 160 - DNA, 60 bp, Artificial Sequence
  Description of Artificial Sequence Synthetic primer
ccatctcatccctgcgtgtctccgactcaggatacgtaaggaatgaccggctgtctgttt

SEQ ID NO: 161 - DNA, 59 bp, Artificial Sequence
  Description of Artificial Sequence Synthetic primer
ccatctcatccctgcgtgtctccgactcagctcggattaaaaagtggcaccaccaacaa

SEQ ID NO: 162 - DNA, 60 bp, Artificial Sequence
  Description of Artificial Sequence Synthetic primer
ccatctcatccctgcgtgtctccgactcagttggattcgtcttgtagcgcttcccacagt

SEQ ID NO: 163 - DNA, 61 bp, Artificial Sequence
  Description of Artificial Sequence Synthetic primer
ccatctcatccctgcgtgtctccgactcagccgtccgctaagcttctttccacaatcctca

SEQ ID NO: 164 - DNA, 67 bp, Artificial Sequence
  Description of Artificial Sequence Synthetic primer
ccatctcatccctgcgtgtctccgactcaggcgattgcaactgtaccccataaatatgtacaacact

SEQ ID NO: 165 - DNA, 58 bp, Artificial Sequence
  Description of Artificial Sequence Synthetic primer
ccatctcatccctgcgtgtctccgactcagccatgcataacgccagatgggtaagtgc

SEQ ID NO: 166 - DNA, 59 bp, Artificial Sequence
  Description of Artificial Sequence Synthetic primer
ccatctcatccctgcgtgtctccgactcagtaattgcaattgcaaatcagtggctctcc

SEQ ID NO: 167 - DNA, 58 bp, Artificial Sequence
  Description of Artificial Sequence Synthetic primer
ccatctcatccctgcgtgtctccgactcagacgactccaactcccttcctgccacctg

SEQ ID NO: 168 - DNA, 65 bp, Artificial Sequence
  Description of Artificial Sequence Synthetic primer
ccatctcatccctgcgtgtctccgactcagatcatgcagaacatgtgatacttttgggaatgaag

SEQ ID NO: 169 - DNA, 62 bp, Artificial Sequence
  Description of Artificial Sequence Synthetic primer
ccatctcatccctgcgtgtctccgactcagaactcctaatcttctgaacaccaaattggaaa

SEQ ID NO: 170 - DNA, 61 bp, Artificial Sequence
  Description of Artificial Sequence Synthetic primer
ccatctcatccctgcgtgtctccgactcagggatattcgttgttaagagcccagaggttca

SEQ ID NO: 171 - DNA, 58 bp, Artificial Sequence
  Description of Artificial Sequence Synthetic primer
ccatctcatccctgcgtgtctccgactcagtcggatgactggggcccctactctttga

SEQ ID NO: 172 - DNA, 58 bp, Artificial Sequence
  Description of Artificial Sequence Synthetic primer
ccatctcatccctgcgtgtctccgactcaggacgcgcgagctcgcagcccctacactg

SEQ ID NO: 173 - DNA, 58 bp, Artificial Sequence
  Description of Artificial Sequence Synthetic primer
ccatctcatccctgcgtgtctccgactcaggcctagaccttgactgccctctgctgtg

SEQ ID NO: 174 - DNA, 60 bp, Artificial Sequence
  Description of Artificial Sequence Synthetic primer
ccatctcatccctgcgtgtctccgactcaggaccaggcgaagtgacgatgagcaggaggt

SEQ ID NO: 175 - DNA, 58 bp, Artificial Sequence
  Description of Artificial Sequence Synthetic primer
ccatctcatccctgcgtgtctccgactcaggctctggcgtgccaattcccattgacca

SEQ ID NO: 176 - DNA, 60 bp, Artificial Sequence
  Description of Artificial Sequence Synthetic primer
ccatctcatccctgcgtgtctccgactcagtggtccggaaccaagcttcctgaaccagac

SEQ ID NO: 177 - DNA, 58 bp, Artificial Sequence
  Description of Artificial Sequence Synthetic primer
ccatctcatccctgcgtgtctccgactcagctctgcgtctctagtgggggcattccaa

SEQ ID NO: 178 - DNA, 58 bp, Artificial Sequence
  Description of Artificial Sequence Synthetic primer
ccatctcatccctgcgtgtctccgactcagccagaagcagctctagggcgcgtttcct

SEQ ID NO: 179 - DNA, 59 bp, Artificial Sequence
  Description of Artificial Sequence Synthetic primer
ccatctcatccctgcgtgtctccgactcagggaaggttgctcagcctttcctcgctcta

SEQ ID NO: 180 - DNA, 60 bp, Artificial Sequence
  Description of Artificial Sequence Synthetic primer
ccatctcatccctgcgtgtctccgactcagtaacggtacgtccacataactcgcttgcag

SEQ ID NO: 181 - DNA, 61 bp, Artificial Sequence
  Description of Artificial Sequence Synthetic primer
ccatctcatccctgcgtgtctccgactcagctcgctcatggaactgtagcccagacactgc

SEQ ID NO: 182 - DNA, 63 bp, Artificial Sequence
  Description of Artificial Sequence Synthetic primer
ccatctcatccctgcgtgtctccgactcagactccaaggcacaaagctggaaactcttcccta

SEQ ID NO: 183 - DNA, 60 bp, Artificial Sequence
  Description of Artificial Sequence Synthetic primer
ccatctcatccctgcgtgtctccgactcaggagctgctatccaacaagcccaacaagttc

SEQ ID NO: 184 - DNA, 60 bp, Artificial Sequence
  Description of Artificial Sequence Synthetic primer
ccatctcatccctgcgtgtctccgactcagcgttgaggccgaatgaccggctgtctgttt

SEQ ID NO: 185 - DNA, 59 bp, Artificial Sequence
  Description of Artificial Sequence Synthetic primer
ccatctcatccctgcgtgtctccgactcagttctggatccaaagtggcaccaccaacaa

SEQ ID NO: 186 - DNA, 60 bp, Artificial Sequence
  Description of Artificial Sequence Synthetic primer
ccatctcatccctgcgtgtctccgactcagccggattccacttgtagcgcttcccacagt

SEQ ID NO: 187 - DNA, 61 bp, Artificial Sequence
  Description of Artificial Sequence Synthetic primer
ccatctcatccctgcgtgtctccgactcagtccatcgcttagcttctttccacaatcctca

SEQ ID NO: 188 - DNA, 67 bp, Artificial Sequence
  Description of Artificial Sequence Synthetic primer
ccatctcatccctgcgtgtctccgactcagttacttctcactgtaccccataaatatgtacaacact

SEQ ID NO: 189 - DNA, 10 bp, Artificial Sequence
  Description of Artificial Sequence Synthetic oligonucleotide
ttaacggacg

SEQ ID NO: 190 - DNA, 10 bp, Artificial Sequence
  Description of Artificial Sequence Synthetic oligonucleotide
tccggcttac

SEQ ID NO: 191 - DNA, 10 bp, Artificial Sequence
  Description of Artificial Sequence Synthetic oligonucleotide
tctcattcag

SEQ ID NO: 192 - DNA, 10 bp, Artificial Sequence
  Description of Artificial Sequence Synthetic oligonucleotide
gcggtcatac

SEQ ID NO: 193 - DNA, 10 bp, Artificial Sequence
  Description of Artificial Sequence Synthetic oligonucleotide
taggacgttc

SEQ ID NO: 194 - DNA, 10 bp, Artificial Sequence
  Description of Artificial Sequence Synthetic oligonucleotide
acgacgcaac

SEQ ID NO: 195 - DNA, 10 bp, Artificial Sequence
  Description of Artificial Sequence Synthetic oligonucleotide
cttctcggac

SEQ ID NO: 196 - DNA, 10 bp, Artificial Sequence
  Description of Artificial Sequence Synthetic oligonucleotide
cattgccgtt

SEQ ID NO: 197 - DNA, 10 bp, Artificial Sequence
  Description of Artificial Sequence Synthetic oligonucleotide
cgagccagaa

SEQ ID NO: 198 - DNA, 10 bp, Artificial Sequence
  Description of Artificial Sequence Synthetic oligonucleotide
tgaggacggc

SEQ ID NO: 199 - DNA, 10 bp, Artificial Sequence
  Description of Artificial Sequence Synthetic oligonucleotide
gcctgcgcag

SEQ ID NO: 200 - DNA, 10 bp, Artificial Sequence
  Description of Artificial Sequence Synthetic oligonucleotide
gttgacgtct

SEQ ID NO: 201 - DNA, 10 bp, Artificial Sequence
  Description of Artificial Sequence Synthetic oligonucleotide
gagatcgatt

SEQ ID NO: 202 - DNA, 10 bp, Artificial Sequence
  Description of Artificial Sequence Synthetic oligonucleotide
agttcgagcc

SEQ ID NO: 203 - DNA, 10 bp, Artificial Sequence
  Description of Artificial Sequence Synthetic oligonucleotide
ctcaggctca

SEQ ID NO: 204 - DNA, 10 bp, Artificial Sequence
  Description of Artificial Sequence Synthetic oligonucleotide
ggcaatataa

SEQ ID NO: 205 - DNA, 10 bp, Artificial Sequence
  Description of Artificial Sequence Synthetic oligonucleotide
ggtactcatg

SEQ ID NO: 206 - DNA, 10 bp, Artificial Sequence
  Description of Artificial Sequence Synthetic oligonucleotide
tctggttcaa

SEQ ID NO: 207 - DNA, 10 bp, Artificial Sequence
  Description of Artificial Sequence Synthetic oligonucleotide
gtcctataag

SEQ ID NO: 208 - DNA, 10 bp, Artificial Sequence
  Description of Artificial Sequence Synthetic oligonucleotide
gtcagcctcc

SEQ ID NO: 209 - DNA, 10 bp, Artificial Sequence
  Description of Artificial Sequence Synthetic oligonucleotide
ttcaagctcg

SEQ ID NO: 210 - DNA, 10 bp, Artificial Sequence
  Description of Artificial Sequence Synthetic oligonucleotide
gtaccagcgc

SEQ ID NO: 211 - DNA, 10 bp, Artificial Sequence
  Description of Artificial Sequence Synthetic oligonucleotide
tcctattcgg

SEQ ID NO: 212 - DNA, 10 bp, Artificial Sequence
  Description of Artificial Sequence Synthetic oligonucleotide
gccagcgatt

SEQ ID NO: 213 - DNA, 10 bp, Artificial Sequence
  Description of Artificial Sequence Synthetic oligonucleotide
acctagactg

SEQ ID NO: 214 - DNA, 10 bp, Artificial Sequence
  Description of Artificial Sequence Synthetic oligonucleotide
actggttcgc

SEQ ID NO: 215 - DNA, 10 bp, Artificial Sequence
  Description of Artificial Sequence Synthetic oligonucleotide
ccatattagg

SEQ ID NO: 216 - DNA, 10 bp, Artificial Sequence
  Description of Artificial Sequence Synthetic oligonucleotide
gctcgtcagc

SEQ ID NO: 217 - DNA, 10 bp, Artificial Sequence
  Description of Artificial Sequence Synthetic oligonucleotide
cgtaatgacg

SEQ ID NO: 218 - DNA, 10 bp, Artificial Sequence
  Description of Artificial Sequence Synthetic oligonucleotide
ccggcgctga

SEQ ID NO: 219 - DNA, 10 bp, Artificial Sequence
  Description of Artificial Sequence Synthetic oligonucleotide
cgcgaagata

SEQ ID NO: 220 - DNA, 10 bp, Artificial Sequence
  Description of Artificial Sequence Synthetic oligonucleotide
gaaccgcaga

SEQ ID NO: 221 - DNA, 10 bp, Artificial Sequence
  Description of Artificial Sequence Synthetic oligonucleotide
ttggcagaga

SEQ ID NO: 222 - DNA, 10 bp, Artificial Sequence
  Description of Artificial Sequence Synthetic oligonucleotide
gcatctctgc

SEQ ID NO: 223 - DNA, 10 bp, Artificial Sequence
  Description of Artificial Sequence Synthetic oligonucleotide
ttggaccgca

SEQ ID NO: 224 - DNA, 10 bp, Artificial Sequence
  Description of Artificial Sequence Synthetic oligonucleotide
gcagaacgtc

SEQ ID NO: 225 - DNA, 10 bp, Artificial Sequence
  Description of Artificial Sequence Synthetic oligonucleotide
aacttcgagc

SEQ ID NO: 226 - DNA, 10 bp, Artificial Sequence
  Description of Artificial Sequence Synthetic oligonucleotide
gctcctagag

SEQ ID NO: 227 - DNA, 10 bp, Artificial Sequence
  Description of Artificial Sequence Synthetic oligonucleotide
tatctagctt

SEQ ID NO: 228 - DNA, 10 bp, Artificial Sequence
  Description of Artificial Sequence Synthetic oligonucleotide
gagtattggc

SEQ ID NO: 229 - DNA, 10 bp, Artificial Sequence
  Description of Artificial Sequence Synthetic oligonucleotide
cctgagctca

SEQ ID NO: 230 - DNA, 10 bp, Artificial Sequence
  Description of Artificial Sequence Synthetic oligonucleotide
caggcgagta

SEQ ID NO: 231 - DNA, 10 bp, Artificial Sequence
  Description of Artificial Sequence Synthetic oligonucleotide
gcaggcagag

SEQ ID NO: 232 - DNA, 10 bp, Artificial Sequence
  Description of Artificial Sequence Synthetic oligonucleotide
gcgtcgatac

SEQ ID NO: 233 - DNA, 10 bp, Artificial Sequence
  Description of Artificial Sequence Synthetic oligonucleotide
cgatgattat

SEQ ID NO: 234 - DNA, 10 bp, Artificial Sequence
  Description of Artificial Sequence Synthetic oligonucleotide
gacggctggc

SEQ ID NO: 235 - DNA, 10 bp, Artificial Sequence
  Description of Artificial Sequence Synthetic oligonucleotide
ggagcctgag

SEQ ID NO: 236 - DNA, 10 bp, Artificial Sequence
  Description of Artificial Sequence Synthetic oligonucleotide
cctgactgct

SEQ ID NO: 237 - DNA, 10 bp, Artificial Sequence
  Description of Artificial Sequence Synthetic oligonucleotide
acggctgacg

SEQ ID NO: 238 - DNA, 10 bp, Artificial Sequence
  Description of Artificial Sequence Synthetic oligonucleotide
taaccatagc

SEQ ID NO: 239 - DNA, 10 bp, Artificial Sequence
  Description of Artificial Sequence Synthetic oligonucleotide
tcttgccttc

SEQ ID NO: 240 - DNA, 10 bp, Artificial Sequence
  Description of Artificial Sequence Synthetic oligonucleotide
ttcttagatt

SEQ ID NO: 241 - DNA, 10 bp, Artificial Sequence
  Description of Artificial Sequence Synthetic oligonucleotide
tcatctcatt

SEQ ID NO: 242 - DNA, 10 bp, Artificial Sequence
  Description of Artificial Sequence Synthetic oligonucleotide
tctccgctcg

SEQ ID NO: 243 - DNA, 10 bp, Artificial Sequence
  Description of Artificial Sequence Synthetic oligonucleotide
tgccatatgc

SEQ ID NO: 244 - DNA, 10 bp, Artificial Sequence
  Description of Artificial Sequence Synthetic oligonucleotide
taaggcctct

SEQ ID NO: 245 - DNA, 10 bp, Artificial Sequence
  Description of Artificial Sequence Synthetic oligonucleotide
gagtaggccg

SEQ ID NO: 246 - DNA, 10 bp, Artificial Sequence
  Description of Artificial Sequence Synthetic oligonucleotide
gcaataagct

SEQ ID NO: 247 - DNA, 10 bp, Artificial Sequence
  Description of Artificial Sequence Synthetic oligonucleotide
ggcgttgcaa

SEQ ID NO: 248 - DNA, 10 bp, Artificial Sequence
  Description of Artificial Sequence Synthetic oligonucleotide
ccaagaagcg

SEQ ID NO: 249 - DNA, 10 bp, Artificial Sequence
  Description of Artificial Sequence Synthetic oligonucleotide
ggttacctcg

SEQ ID NO: 250 - DNA, 10 bp, Artificial Sequence
  Description of Artificial Sequence Synthetic oligonucleotide
ctccgcctta

SEQ ID NO: 251 - DNA, 10 bp, Artificial Sequence
  Description of Artificial Sequence Synthetic oligonucleotide
ctccagagat

SEQ ID NO: 252 - DNA, 10 bp, Artificial Sequence
  Description of Artificial Sequence Synthetic oligonucleotide
gtcgaggtag

SEQ ID NO: 253 - DNA, 10 bp, Artificial Sequence
  Description of Artificial Sequence Synthetic oligonucleotide
tatggacctg

SEQ ID NO: 254 - DNA, 10 bp, Artificial Sequence
  Description of Artificial Sequence Synthetic oligonucleotide
tacctgctag

SEQ ID NO: 255 - DNA, 10 bp, Artificial Sequence
  Description of Artificial Sequence Synthetic oligonucleotide
ccgcgaccga

SEQ ID NO: 256 - DNA, 10 bp, Artificial Sequence
  Description of Artificial Sequence Synthetic oligonucleotide
gttgaacgtt

SEQ ID NO: 257 - DNA, 10 bp, Artificial Sequence
  Description of Artificial Sequence Synthetic oligonucleotide
tgccaacgca

SEQ ID NO: 258 - DNA, 10 bp, Artificial Sequence
  Description of Artificial Sequence Synthetic oligonucleotide
ggattgacct

SEQ ID NO: 259 - DNA, 10 bp, Artificial Sequence
  Description of Artificial Sequence Synthetic oligonucleotide
ggacggattc

SEQ ID NO: 260 - DNA, 10 bp, Artificial Sequence
  Description of Artificial Sequence Synthetic oligonucleotide
tcctccgtcg

SEQ ID NO: 261 - DNA, 10 bp, Artificial Sequence
  Description of Artificial Sequence Synthetic oligonucleotide
agttcatggt

SEQ ID NO: 262 - DNA, 10 bp, Artificial Sequence
  Description of Artificial Sequence Synthetic oligonucleotide
tatccattcc

SEQ ID NO: 263 - DNA, 10 bp, Artificial Sequence
  Description of Artificial Sequence Synthetic oligonucleotide
ggagagcgcg

SEQ ID NO: 264 - DNA, 10 bp, Artificial Sequence
  Description of Artificial Sequence Synthetic oligonucleotide
cggaccttgg

SEQ ID NO: 265 - DNA, 10 bp, Artificial Sequence
  Description of Artificial Sequence Synthetic oligonucleotide
ggcaatctcc

SEQ ID NO: 266 - DNA, 10 bp, Artificial Sequence
  Description of Artificial Sequence Synthetic oligonucleotide
aggattgatt

SEQ ID NO: 267 - DNA, 10 bp, Artificial Sequence
  Description of Artificial Sequence Synthetic oligonucleotide
gccgttgcct

SEQ ID NO: 268 - DNA, 10 bp, Artificial Sequence
  Description of Artificial Sequence Synthetic oligonucleotide
aagtacgtcg

SEQ ID NO: 269 - DNA, 10 bp, Artificial Sequence
  Description of Artificial Sequence Synthetic oligonucleotide
tggcttaagg

SEQ ID NO: 270 - DNA, 10 bp, Artificial Sequence
  Description of Artificial Sequence Synthetic oligonucleotide
ctcttccaga

SEQ ID NO: 271 - DNA, 10 bp, Artificial Sequence
  Description of Artificial Sequence Synthetic oligonucleotide
cgttcttcaa

SEQ ID NO: 272 - DNA, 10 bp, Artificial Sequence
  Description of Artificial Sequence Synthetic oligonucleotide
caacggctgc

SEQ ID NO: 273 - DNA, 10 bp, Artificial Sequence
  Description of Artificial Sequence Synthetic oligonucleotide
gcaagtaacc

SEQ ID NO: 274 - DNA, 10 bp, Artificial Sequence
  Description of Artificial Sequence Synthetic oligonucleotide
gttcatagtc

SEQ ID NO: 275 - DNA, 10 bp, Artificial Sequence
  Description of Artificial Sequence Synthetic oligonucleotide
acggcgagcc

SEQ ID NO: 276 - DNA, 10 bp, Artificial Sequence
  Description of Artificial Sequence Synthetic oligonucleotide
gtatggtcgg

SEQ ID NO: 277 - DNA, 10 bp, Artificial Sequence
  Description of Artificial Sequence Synthetic oligonucleotide
tcggttatcc

SEQ ID NO: 278 - DNA, 10 bp, Artificial Sequence
  Description of Artificial Sequence Synthetic oligonucleotide
gcggtcgata

SEQ ID NO: 279 - DNA, 10 bp, Artificial Sequence
  Description of Artificial Sequence Synthetic oligonucleotide
tcctcagtat

SEQ ID NO: 280 - DNA, 10 bp, Artificial Sequence
  Description of Artificial Sequence Synthetic oligonucleotide
accgttcctg

SEQ ID NO: 281 - DNA, 10 bp, Artificial Sequence
  Description of Artificial Sequence Synthetic oligonucleotide
gcctgctctt

SEQ ID NO: 282 - DNA, 10 bp, Artificial Sequence
  Description of Artificial Sequence Synthetic oligonucleotide
agcgtaacca

SEQ ID NO: 283 - DNA, 10 bp, Artificial Sequence
  Description of Artificial Sequence Synthetic oligonucleotide
ttgcctgatg

SEQ ID NO: 284 - DNA, 10 bp, Artificial Sequence
  Description of Artificial Sequence Synthetic oligonucleotide
ttattgatct

SEQ ID NO: 285 - DNA, 10 bp, Artificial Sequence
  Description of Artificial Sequence Synthetic oligonucleotide
tacgctcgga

SEQ ID NO: 286 - DNA, 10 bp, Artificial Sequence
  Description of Artificial Sequence Synthetic oligonucleotide
caatccaagg

SEQ ID NO: 287 - DNA, 10 bp, Artificial Sequence
  Description of Artificial Sequence Synthetic oligonucleotide
tcgtagctat

SEQ ID NO: 288 - DNA, 10 bp, Artificial Sequence
  Description of Artificial Sequence Synthetic oligonucleotide
cgctcatcgc

SEQ ID NO: 289 - DNA, 10 bp, Artificial Sequence
  Description of Artificial Sequence Synthetic oligonucleotide
tccgttcatt

SEQ ID NO: 290 - DNA, 10 bp, Artificial Sequence
  Description of Artificial Sequence Synthetic oligonucleotide
cggccaggct

SEQ ID NO: 291 - DNA, 10 bp, Artificial Sequence
  Description of Artificial Sequence Synthetic oligonucleotide
caacctatct

SEQ ID NO: 292 - DNA, 10 bp, Artificial Sequence
  Description of Artificial Sequence Synthetic oligonucleotide
cgtaatctca

SEQ ID NO: 293 - DNA, 10 bp, Artificial Sequence
  Description of Artificial Sequence Synthetic oligonucleotide
atatcgcgac

SEQ ID NO: 294 - DNA, 10 bp, Artificial Sequence
  Description of Artificial Sequence Synthetic oligonucleotide
tcaatatctg

SEQ ID NO: 295 - DNA, 10 bp, Artificial Sequence
  Description of Artificial Sequence Synthetic oligonucleotide
atagagtata

SEQ ID NO: 296 - DNA, 10 bp, Artificial Sequence
  Description of Artificial Sequence Synthetic oligonucleotide
gcaactagtt

SEQ ID NO: 297 - DNA, 10 bp, Artificial Sequence
  Description of Artificial Sequence Synthetic oligonucleotide
atctcgaatc

SEQ ID NO: 298 - DNA, 10 bp, Artificial Sequence
  Description of Artificial Sequence Synthetic oligonucleotide
ccaggagcga

SEQ ID NO: 299 - DNA, 10 bp, Artificial Sequence
  Description of Artificial Sequence Synthetic oligonucleotide
atctccatcg

SEQ ID NO: 300 - DNA, 10 bp, Artificial Sequence
  Description of Artificial Sequence Synthetic oligonucleotide
ttgacgagct

SEQ ID NO: 301 - DNA, 10 bp, Artificial Sequence
  Description of Artificial Sequence Synthetic oligonucleotide
tactattacc

SEQ ID NO: 302 - DNA, 10 bp, Artificial Sequence
  Description of Artificial Sequence Synthetic oligonucleotide
cgtcctggac

SEQ ID NO: 303 - DNA, 10 bp, Artificial Sequence
  Description of Artificial Sequence Synthetic oligonucleotide
ctcggcgctt

SEQ ID NO: 304 - DNA, 10 bp, Artificial Sequence
  Description of Artificial Sequence Synthetic oligonucleotide
gatacgtaag

SEQ ID NO: 305 - DNA, 10 bp, Artificial Sequence
  Description of Artificial Sequence Synthetic oligonucleotide
ctcggattaa

SEQ ID NO: 306 - DNA, 10 bp, Artificial Sequence
  Description of Artificial Sequence Synthetic oligonucleotide
ttggattcgt

SEQ ID NO: 307 - DNA, 10 bp, Artificial Sequence
  Description of Artificial Sequence Synthetic oligonucleotide
ccgtccgcta

SEQ ID NO: 308 - DNA, 10 bp, Artificial Sequence
  Description of Artificial Sequence Synthetic oligonucleotide
gcgattgcaa

SEQ ID NO: 309 - DNA, 10 bp, Artificial Sequence
  Description of Artificial Sequence Synthetic oligonucleotide
ccatgcataa

SEQ ID NO: 310 - DNA, 10 bp, Artificial Sequence
  Description of Artificial Sequence Synthetic oligonucleotide
taattgcaat

SEQ ID NO: 311 - DNA, 10 bp, Artificial Sequence
  Description of Artificial Sequence Synthetic oligonucleotide
acgactccaa

SEQ ID NO: 312 - DNA, 10 bp, Artificial Sequence
  Description of Artificial Sequence Synthetic oligonucleotide
atcatgcaga

SEQ ID NO: 313 - DNA, 10 bp, Artificial Sequence
  Description of Artificial Sequence Synthetic oligonucleotide
aactcctaat

SEQ ID NO: 314 - DNA, 10 bp, Artificial Sequence
  Description of Artificial Sequence Synthetic oligonucleotide
ggatattcgt

SEQ ID NO: 315 - DNA, 10 bp, Artificial Sequence
  Description of Artificial Sequence Synthetic oligonucleotide
tcggatgact

SEQ ID NO: 316 - DNA, 10 bp, Artificial Sequence
  Description of Artificial Sequence Synthetic oligonucleotide
gacgcgcgag

SEQ ID NO: 317 - DNA, 10 bp, Artificial Sequence
  Description of Artificial Sequence Synthetic oligonucleotide
gcctagacct

SEQ ID NO: 318 - DNA, 10 bp, Artificial Sequence
  Description of Artificial Sequence Synthetic oligonucleotide
gaccaggcga

SEQ ID NO: 319 - DNA, 10 bp, Artificial Sequence
  Description of Artificial Sequence Synthetic oligonucleotide
gctctggcgt

SEQ ID NO: 320 - DNA, 10 bp, Artificial Sequence
  Description of Artificial Sequence Synthetic oligonucleotide
tggtccggaa

SEQ ID NO: 321 - DNA, 10 bp, Artificial Sequence
  Description of Artificial Sequence Synthetic oligonucleotide
ctctgcgtct

SEQ ID NO: 322 - DNA, 10 bp, Artificial Sequence
  Description of Artificial Sequence Synthetic oligonucleotide
ccagaagcag

SEQ ID NO: 323 - DNA, 10 bp, Artificial Sequence
  Description of Artificial Sequence Synthetic oligonucleotide
ggaaggttgc

SEQ ID NO: 324 - DNA, 10 bp, Artificial Sequence
  Description of Artificial Sequence Synthetic oligonucleotide
taacggtacg

SEQ ID NO: 325 - DNA, 10 bp, Artificial Sequence
  Description of Artificial Sequence Synthetic oligonucleotide
ctcgctcatg

SEQ ID NO: 326 - DNA, 10 bp, Artificial Sequence
  Description of Artificial Sequence Synthetic oligonucleotide
actccaaggc

SEQ ID NO: 327 - DNA, 10 bp, Artificial Sequence
  Description of Artificial Sequence Synthetic oligonucleotide
gagctgctat

SEQ ID NO: 328 - DNA, 10 bp, Artificial Sequence
  Description of Artificial Sequence Synthetic oligonucleotide
cgttgaggcc

SEQ ID NO: 329 - DNA, 10 bp, Artificial Sequence
  Description of Artificial Sequence Synthetic oligonucleotide
ttctggatcc

SEQ ID NO: 330 - DNA, 10 bp, Artificial Sequence
  Description of Artificial Sequence Synthetic oligonucleotide
ccggattcca

SEQ ID NO: 331 - DNA, 10 bp, Artificial Sequence
  Description of Artificial Sequence Synthetic oligonucleotide
tccatcgctt

SEQ ID NO: 332 - DNA, 10 bp, Artificial Sequence
  Description of Artificial Sequence Synthetic oligonucleotide
ttacttctca

SEQ ID NO: 333 - DNA, 73 bp, Homo sapiens
aaatgcactctgatatgggcctaatcatataatgccaatctcccagggtccaaagggtgatgccggtgcacct

SEQ ID NO: 334 - protein, 9 aa, Homo sapiens
GPKGDAGAP

SEQ ID NO: 335 - DNA, 15 bp, Homo sapiens
tttggcagtgtcttc

SEQ ID NO: 336 - DNA, 100 bp, Homo sapiens
ggcaactctgtagatctaggatgttcggtcagcgattcattcttttcttctaaagatttttccaatttggtgttcagaagattattttttagtaagttat

SEQ ID NO: 337 - protein, 34 aa, Homo sapiens
NNLLKNNLLNTKLEKSLEEKNESLTEHPRSTELP

SEQ ID NO: 338 - DNA, 36 bp, Homo sapiens
ttgcgccccgggctgcccgccctgccggctgccccc

SEQ ID NO: 339 - protein, 12 aa, Homo sapiens
GGSRQGGQPGAQ